(19) United States
(12) Patent Application Publication
(10) Pub. No.: US 2008/0044857 A1
(43) Pub. Date: Feb. 21, 2008

(54) Methods For Making And Using Mass Tag Standards For Quantitative Proteomics

(75) Inventor: David E. Anderson, Washington, DC (US)

Correspondence Address: KLARQUIST SPARKMAN, LLP, 121 S.W. SALMON STREET, SUITE #1600, PORTLAND, OR, 97204-2988, US

(73) Assignee: THE GOV OF USA as represented by THE SECRETARY OF, Rockville, MD (US)

(21) Appl. No.: 11/597,596

(22) PCT Filed: May 25, 2005
(86) PCT No.: PCT/US05/18459

§ 371 Date: Nov. 22, 2006

Related U.S. Application Data
(60) Provisional application No. 60/574,612, filed on May 25, 2004.

(51) Int. Cl. G01N 33/68; C07K 2/00; C12P 21/00

(52) U.S. Cl. 435/71.1; 436/86; 530/300

(57) ABSTRACT

Disclosed are methods for producing peptide standards for quantitative proteomics. In one disclosed embodiment, chimeric polypeptides that are a combination of mass tags for multiple proteins are expressed in a host cell that is grown on an isotopically-altered medium. The mass tags in the chimeric polypeptide are separated by specific cleavage sites (such as trypsin cleavage sites), and upon treatment with an appropriate protein cleavage agent (such as trypsin) the constituent peptide standards are released. Methods of mass spectrometric analysis that employ the disclosed chimeric polypeptides (or the peptide standards liberated therefrom) also are disclosed.

### PRIORITY CLAIM

This application claims the benefit of U.S. Provisional Patent Application No. 60/574,612 filed May 25, 2004, which is incorporated herein by reference in its entirety.

### FIELD

This invention relates to methods for quantitative proteomics. More specifically, this invention relates to methods for making mass tag standards and their use in quantitative mass spectrometric analyses of proteins.

### BACKGROUND

Genomic technology has advanced to a point where it is possible to determine complete genomic sequences and to quantitatively measure the mRNA levels for each gene expressed in a cell. However, proteins control and execute virtually every biological process, and protein expression levels and protein activity are not directly apparent from the corresponding gene sequence, or even the expression level of the corresponding mRNA transcript. Therefore, a complete description of a biological system includes the identity, quantity and state of activity of the proteins which constitute the biological system. Analysis of the proteins expressed by a cell is termed proteome analysis, or proteomics.

At present, no proteomic technology approaches the high-throughput and level of automation of genomic technology. Two-dimensional gel electrophoresis (2DE) has been the dominant technique for assessing large-scale changes in protein expression patterns because it permits analysis of several hundred proteins simultaneously. Each separated spot on a labeled (or stained) 2DE gel typically represents a single protein. Therefore, it is possible to determine the relative expression levels of proteins in cells subjected to different states (such as a stressed state and a non-stressed state) by comparing the spot intensities of the proteins on independent gels that are prepared for cell samples from the different states. Unfortunately, proteins do not migrate to reproducible positions on gels, and it is therefore necessary to identify the proteins responsible for each spot before quantitative comparisons can be made between gels. The need to identify the protein responsible for a given spot on the 2DE gel generally precludes automation of the technique and results in low through-put.

Spot identification in two-dimensional gels has been greatly simplified by the development of biological mass spectrometry (MS). In this method, individual protein spots on two-dimensional gels are excised and digested with an endoprotease (typically trypsin) to generate a mixture of peptides that is then subjected to mass spectral analysis (see, for example, Romijn et al., J. Chrom. A 1000:589-608, 2003). The pattern of masses observed for the mixture of peptides provides a fingerprint of the protein responsible for the spot. The observed fingerprint pattern is then compared to a database of fingerprint patterns expected (or known) to result from endoprotease cleavage of known proteins, and an identification of the protein is made based on similarities of its pattern to a database pattern. In some instances, a single peptide mass signal will identify a particular protein, and in others, a set of mass signals for peptides and perhaps fragments thereof are needed to unambiguously identify a protein. Whether a single peptide or a set of peptides are needed to identify a protein depends in part upon the mass resolution of the mass spectrometer used. Increases in mass resolution make it more likely that it will be possible to identify a protein with a single peptide mass signal. The peptide or peptides that are uniquely characteristic of a protein are termed a “mass tag,” and the use of mass patterns to identify proteins is often referred to as “mass mapping.”

Quantitation of proteins by mass spectrometry is possible if isotopically-defined standards are employed. Peptides that are labeled with stable heavy isotopes (for example, ²H, ¹³C, ¹⁵N, and/or ¹⁸O) provide mass signals that are separated from and may be compared to the mass signals of their non-labeled counterparts. The ratio of the intensities of the mass signals in a mass spectrum that are due to a labeled peptide and to its non-labeled counterpart provides a measure of the relative concentrations of each in the sample. If the absolute concentration of either is known, the concentration of the other may be calculated using the ratio of their signal intensities. Isotopically-labeled peptides may be derived either from the sample (for example, a sample obtained from a cell grown in an isotopically-altered medium) or from the standards (for example, isotopically labeled peptides expressed as chimera according to the disclosed methods).

Stable isotopes may be incorporated into peptides either biologically (in vivo) or chemically (in vitro). Biological isotopic labeling schemes for quantitative mass spectrometry include stable isotope labeling by amino acids in cell culture (SILAC) and related techniques (see, for example, Ong et al., Molecular & Cellular Proteomics, 1.5:376-386, 2002). In the SILAC technique, isotopically-labeled amino acids are added to an amino acid deficient cell culture, and are incorporated into proteins during cell growth. Cells that are grown in an isotopically-altered medium and subjected to a first state (such as a stressed state) are mixed with cells that are grown on a non-isotopically-altered medium and subjected to a second state (such as a non-stressed state). The proteins in the mixture are digested with an endoprotease (such as trypsin) and a mass spectrum is obtained. Pairs of mass signals corresponding to the labeled and non-labeled versions of the endoprotease cleavage peptides are identified. The ratio of the relative intensities of the signals in each pair is a measure of the relative concentrations of the proteins in the cells subjected to the two states.

Chemical labeling schemes include the isotope-coded affinity tag (ICAT) method and related techniques (see, for example, Gygi et al., Nat. Biotechnol., 17: 994-999, 1999 and Lill, Mass Spectrometry Reviews, 22: 182-194, 2003). In the ICAT scheme, isotopically-labeled reagent molecules that react with specific amino acid residues are added to a sample from cells in one state, and counterpart non-isotopically labeled reagent molecules are reacted with a sample from cells in a second state. The two samples are digested with an endoprotease and mixed (or mixed and digested) for mass spectral analysis. The mass spectral signal intensities for the heavy and light versions of the peptides are used to provide a measure of the relative level of the proteins in the two states. Again, only relative quantitation is provided.

Protein expression levels may be absolutely quantified by mass spectrometry if endoprotease cleavage peptide standards of known concentration are available. Typically, the standards are isotopically labeled peptides, and these are added in known amounts to a non-labeled protein sample that has been digested with an endoprotease. The combined sample is analyzed by mass spectrometry, and the ratios of the mass spectral signal intensities for the labeled peptide standards and the sample peptides are measured. Since the concentrations of the standard peptides are known, the concentration of the sample peptides (and the proteins they are derived from) may be calculated using the ratios. Isotopically-labeled peptide standards of known concentration are generally synthesized from isotopically labeled amino acids in an expensive process that requires dedicated instrumentation, ultrapure isotopically-labeled reagents, and post-synthesis purification and quantitation via high performance liquid chromatography (HPLC).

An alternative method for providing isotopically-labeled peptide standards is to express the peptides in a host cell grown on an isotopically-altered medium. However, direct expression of peptides in vivo has met with limited success because peptides are generally unstable (see, for example, Lindhout et al., Protein Science, 12: 1786-1791, 2003). One solution to the stability problem has been to express peptides in a fusion construct with a larger fusion protein partner. The fusion protein approach has been applied to produce a single peptide or a few copies of a single peptide (a homopolymer of peptides) as part of the fusion protein (see, Jones et al., Biochemistry, 39: 1870-1878, 2000; Majerle et al., J. Biomol. NMR, 18: 145-151, 2000; Sharon et al., Protein Expr. Purif., 24: 374-383, 2002; and Lindhout et al., Protein Science, 12: 1786-1791, 2003). A large amount of valuable isotopically-labeled amino acids are wasted in producing the large, typically over-expressed, fusion protein, of which only a small part (the peptide portion of the construct) is desired. Furthermore, production of a different fusion construct for each desired peptide is very time-consuming.

### SUMMARY OF THE DISCLOSURE

Methods are disclosed for efficiently producing peptide standards for quantitative proteomics and for using the standards to quantify proteins in a sample. Peptide standards for multiple different proteins of interest are produced in parallel by a method that includes expressing peptide standard sequences for two or more different proteins as a chimeric polypeptide. The chimeric polypeptide includes the standard peptide sequences and one or more cleavage sites between these sequences where the chimeric polypeptide can be selectively cleaved by a protein cleavage agent to liberate the standard peptides it contains. The standard peptides are mass tag sequences for the multiple different proteins of interest, and are produced in a variety of ways to have different masses than corresponding mass tag sequences for the proteins of interest that may be present in a sample. The mass difference between a mass tag sequence liberated from the chimeric polypeptide and a corresponding mass tag sequence liberated from a sample protein make each distinctly detectable by mass spectrometry, so that mass signals for each can be compared and used to quantify the sample proteins.

One advantage of the chimeric polypeptides that contain multiple peptide standards is that they facilitate low-cost, simultaneous analysis and quantitation of many different proteins. For example, at least 10, 20, 30 or 50 different constituent proteins, such as at least 100 such constituent proteins, or at least 1,000 such proteins may be simultaneously analyzed and quantified in a single sample using the appropriate chimeric polypeptide. Additionally, use of the disclosed chimeric polypeptides offers a means to monitor the efficiency of the protein cleavage step used to derive peptides from the sample proteins. This may be accomplished by monitoring for the presence of peptide sequences that are produced by partial cleavage of an added chimeric polypeptide.

Proteins of interest in a sample are quantified by adding a known amount (or concentration) of a disclosed chimeric polypeptide (or standard peptides liberated from a known amount of the chimeric polypeptide) to a sample in a known amount (or concentration). Sample proteins are cleaved by a protein cleavage agent (at the same time and together with the chimeric polypeptide, or separately) to generate sample peptides that correspond in amino acid sequence to the standard peptides. Either the standard peptides or the sample peptides are isotopically-labeled with one or more heavy stable isotopes of pre-selected targets so that each standard peptide and its corresponding sample peptide have different masses and are distinctly detectable by mass spectrometry. A mass spectrum of a sample containing both sample peptides and the added standard peptides typically includes one or more pairs of separated signals that are due to a sample peptide and its corresponding standard peptide. The ratio of the intensity of the signals in each pair reflects the relative amounts (or concentrations) of each peptide present in the sample. Since the amount (or concentration) of the standard peptide is known, the amount (or concentration) of the sample peptide can be calculated by multiplying the ratio of the intensity of the signal for the sample peptide to the intensity of the signal for the standard peptide by the known amount (or concentration) of the standard peptide. Furthermore, since the sample peptides are present in amounts (or concentrations) that are the same as (or related by a known ratio to) the amounts (or concentrations) of the proteins originally in the sample, a determination of the amounts (or concentrations) of the sample peptides also permits a determination of the amounts (or concentrations) of the proteins in the sample.

Labeling of either the standard peptides or the sample peptides with stable heavy isotopes to provide a difference in mass between them can be accomplished by a variety of methods. One method is to express either the chimeric polypeptide or the sample proteins (but not both) in a cell grown on a medium that includes a heavy stable isotope that is incorporated into the peptides as the cell grows. Alternatively, a difference in mass between the standard peptides and the corresponding sample peptides can be provided by covalent modification. In this method, the standard peptides and the corresponding sample peptides are separately reacted (either as the separated peptides or as part of the chimeric polypeptide and the sample proteins, respectively) with different versions of a reagent that have different masses. Typically, one version of the reagent has a different mass from the other because it includes one or more heavy stable isotopes that are not present in the other version.

In some embodiments, peptide standards are labeled with heavy stable isotopes (for example, ¹³C, ¹⁵N, or ¹⁸O) and used for quantitative analysis of unlabeled samples. Peptide standards can be isotopically-labeled by expressing a chimeric polypeptide sequence that includes the peptide standard sequences in a host cell, where the host cell is grown in the presence of one or more heavy stable isotopes that become incorporated into the chimeric polypeptide during growth of the host cell. For example, isotopically-labeled amino acids can be added to a growth medium for the host cell and these amino acids become incorporated into the chimeric polypeptide, and hence into its constituent peptide standards. In other embodiments, unlabeled peptide standards are produced as a chimeric polypeptide and used for quantitative analysis of protein samples that have been labeled with heavy stable isotopes, for example, by growing a cell from which the protein sample is derived on a growth medium enriched in one or more heavy stable isotopes that become incorporated into the sample proteins. In yet other embodiments, both an unlabeled chimeric polypeptide including the peptide standards and the unlabeled sample proteins are separately covalently modified (either before or after cleavage into their constituent peptide standards and corresponding sample peptides, respectively) with different reagents that are isotopic analogs (that is they have the same chemical formula, but different masses) of each other. In any case, isotopic-labeling of the standard peptides or the sample peptides offsets the masses of the standard and sample derived peptides so that mass spectrometric analysis yields separate, distinct signals for each that can be used to quantify sample proteins.

Also disclosed are methods for high-throughput quantitative mass spectrometric analysis of protein samples using peptide standards that are expressed as chimeric polypeptides. In one embodiment, a known amount of an isotopically-labeled chimeric polypeptide is mixed with a protein sample and the mixture is treated with an endoprotease to liberate labeled peptide standards from the chimeric polypeptide and corresponding unlabeled peptides from the sample proteins. Following endoprotease treatment, the mixture is analyzed by mass spectrometry to provide a mass spectrum. Proteins of interest are identified from the masses of their constituent peptides (their mass tags), which appear as one or more mass signals at particular mass-to-charge ratios in the mass spectrum. Mass spectral signals for corresponding isotopically-labeled versions of the mass tag peptides are identified in the mass spectrum (such as based on the mass spectral shift caused by isotopic labeling), and the ratios of the mass signal intensities for the labeled and non-labeled versions of the mass tag peptides are determined. The ratios determined from the mass spectrum are used along with the known amount of the isotopically-labeled standards (from the chimeric polypeptide) to calculate the absolute amounts (or concentrations) of the sample peptides, and thus, the amounts of the proteins of interest in the sample. In some instances, where the mass tag for a protein of interest includes multiple peptides appearing at different mass-to-charge-ratios, the ratios of signals for each unlabeled peptide from the protein in the sample and the corresponding labeled version of the mass tag from the chimeric polypeptide are averaged to provide an average ratio that may be used to calculate the amount (concentration) of the peptides, and thus, the protein of interest. In another embodiment, the sample is isotopically-labeled and unlabeled peptide standards are employed in the method, where the ratio of the mass signal intensities for the labeled sample mass tag peptides and the unlabeled standard peptides is used to quantify sample proteins. In yet other embodiments, sample peptides and standard peptides from a chimeric polypeptide (or the sample proteins and the chimeric polypeptide) are separately reacted with different covalent modification reagents to provide sample and standard peptides having the same sequence and structure, but different masses. The reacted sample and standard peptides are mixed for analysis, but can be distinctly detected in a mass spectrum.

While simple mixtures of proteins can be examined using the disclosed methods, one advantage of the methods is that they enable simultaneous analysis and quantitation of many different proteins. For example, at least 10, 20, 30 or 50 different constituent proteins, such as at least 100 such constituent proteins, or at least 1,000 such proteins may be simultaneously analyzed and quantified in a single sample using the disclosed methods. In addition, concentrations of peptide standards that are provided as combinations in a chimeric polypeptide may be more accurately determined by spectrophotometry than individual peptide standards since the chimeric polypeptide will typically have a higher molar absorptivity than any of its constituent peptides alone. Where the chimeric polypeptide includes mass tags with no, or low, molar absorptivity, a sequence that is rich in UV-absorbing amino acids may be conveniently added to the chimeric polypeptide to increase its molar absorptivity.

In addition, use of the disclosed chimeric polypeptides offers a means to monitor the efficiency of the protein cleavage step used to derive peptides from the sample proteins. This may be accomplished by monitoring for the presence of peptide sequences that are produced by partial cleavage of an added chimeric polypeptide. Mass spectral peaks that correspond to incompletely-cleaved chimeric polypeptide will be evident in a mass spectrum if the cleavage process is not completed, and the strength of such mass spectral peaks is a measure of the amount of uncleaved chimeric polypeptide left in the sample after the protein cleavage step and therefore the efficiency of the cleavage step. For example, if a mass-spectral peak for an un-cleaved chimeric polypeptide is detected in a mass spectrum, a longer period of treatment with a protein cleavage agent may then be used for subsequent samples. Alternatively, the mass spectral peaks for incompletely cleaved chimeric polypeptides may be detected, their presence indicating partial cleavage by the protein cleavage agent.

These and other embodiments and advantages are disclosed in the detailed description of this specification. Additional features and advantages of the disclosed methods will become apparent from the following detailed description of the disclosed embodiments and the claims.

### BRIEF DESCRIPTION OF THE DRAWINGS

FIG. 1 is a diagram shows a generalized embodiment of a method that employs the disclosed chimeric polypeptides to quantify sample proteins.

FIG. 2 is a diagram outlining an exemplary procedure for designing a disclosed chimeric polypeptide.

FIG. 3 is a diagram outlining a exemplary procedure for cloning of a nucleic acid sequence coding for a disclosed chimeric polypeptide.

FIG. 4 is a diagram outlining a exemplary procedure for using a disclosed chimeric polypeptide to provide standards for quantitative analysis of a protein.

FIG. 5 is a base-peak intensity trace of an LC/MS experiment using a set of designed peptides constructed to examine a variety of outcomes that can occur when using a robotic in-gel digester, followed by LC/MS/MS to do protein identification. Fragments T1-T7 (with their respective residue numbers) are the result of digesting the designed polypeptide (SEQ ID NO: 26) with trypsin; the theoretical mass of each peptide fragment is also shown.

FIG. 6 is a pair of detailed mass spectra illustrating the sequence verified position of a peptide that originates from the Asp-Pro bond cleavage (residues 6 and 7) in the T2 peptide (SEQ ID NO: 20). FIG. 6A is the product of spectral summation of an approximate 30 second interval of data obtained while the peptide was eluting into the mass spectrometer. The identity of the peptide was determined by MS/MS data to originate from Asp-Pro bond cleavage (residues 6 and 7) in the T2 peptide (SEQ ID NO: 20). FIG. 6B is a simulation of the expected abundance of different peaks expected in the mass spectrum. The PINGFIYYTTYTYTK peptide (residues 7-21 of SEQ ID NO: 20) is a result of Asp-Pro bond cleavage and the difference between the observed and predicted mass spectra is due to asparagines deamidation.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 shows the amino acid sequence of adenylosuccinate synthetase.

SEQ ID NO: 2 shows the amino acid sequence of AMP deaminase.

SEQ ID NO: 3 shows the amino acid sequence of adenylosuccinate lyase.

SEQ ID NOs: 4-15 shown the amino acid sequences of exemplary mass tags for the purine nucleotide cycle enzymes.

SEQ ID NO: 16 shows the amino acid sequence of a chimeric polypeptide.

SEQ ID NO: 17 shows the nucleic acid sequence produced by back translation of the amino acid sequence shown in SEQ ID NO: 16.

SEQ ID NO: 18 shows the nucleic acid sequence of the back translation product (SEQ ID NO: 17) following E. coli codon optimization.

SEQ ID NO: 19 shows the nucleic acid sequence of the back translation product with E. coli codon optimization (SEQ ID NO: 18) with additional 5′ cloning sequences.

SEQ ID NOs: 20-24 shown the amino acid sequences of a series of designed peptides.

SEQ ID NO: 25 shows the nucleic acid sequence of a synthetic gene encoding a designed chimeric polypeptide.

SEQ ID NO: 26 shows the amino acid sequence of the expressed chimeric protein, with some vector originating sequence.

### DETAILED DESCRIPTION

I.

Abbreviations

| | |
|---|---|
| 2DE: | two-dimensional gel electrophoresis |
| AMT: | accurate mass tag |
| APCI: | atmospheric pressure chemical ionization |
| BB: | Bull-Breese-peptide hydrophobicity parameter. |
| CAD: | collision-activated dissociation |
| CI: | chemical ionization |
| CID: | collision-induced dissociation |
| ESI: | Electrospray Ionization |
| FAB: | fast atom Bombardment |
| FT-ICR: | Fourier transform ion cyclotron resonance |
| GC: | gas chromatography |
| HPLC: | high performance liquid chromatography |
| ICAT: | isotope-coded affinity tag |
| IMAC: | immobilized-metal affinity chromatography |
| IPTG: | isopropylthiogalactoside |
| LC: | liquid chromatography |
| MALDI: | Matrix Assisted Laser Desorption/Ionization |
| MS: | mass spectrometry |
| MS/MS: | tandem MS |
| m/z: | mass-to-charge ratio |
| PCR: | polymerase chain reaction |
| RF: | radio-frequency |
| RP-LC: | reverse phase liquid chromatography |
| SELDI: | Surface Enhanced Laser Desorption/Ionization |
| SILAC: | stable isotope labeling by amino acids in cell culture |
| TOF: | time-of-flight |

II. Explanation of Terms

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341), and George P. Rédei, Encyclopedic Dictionary of Genetics, Genomics, and Proteomics, 2nd Edition, 2003 (ISBN: 0-471-26821-6).

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B. It is further to be understood that all base sizes or amino acid sizes, and all molecular weight or molecular mass values, given for nucleic acids or polypeptides are approximate, and are provided for descriptive purposes. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

In order to facilitate review of the various embodiments of the invention, the following explanations of specific terms are provided:

Affinity tag or sequence: An amino acid sequence added to a recombinant protein to facilitate its purification. Examples of affinity tags (sequences) include, for example, histidine tags (such as 6×His), calmodulin-binding peptide (CBP) and glutathione-S-transferase (GST). Typically, but not always, purification of affinity tagged-proteins takes place in a column containing an affinity resin corresponding to the affinity tag. Purification of 6× His-tagged proteins, for example, takes advantage of the high affinity and specificity of immobilized metal ions (such as, nickel) for histidine residues. In another example, CBP-tagged proteins are purified using a calmodulin resin in the presence of low concentrations of calcium. Glutathione-S-transferase-tagged proteins, an additional example of protein purification, relies on the high affinity and specificity with which GST binds immobilized glutathione.

Chimeric Polypeptide: The term “chimeric polypeptide” refers to a polypeptide that includes a combination of peptide sequences that are found in two or more different proteins or fragments of proteins. The peptide sequences included in a chimeric polypeptide can be from naturally-occurring proteins or non-naturally-occurring proteins, and can be from the same organism or different organisms. However, the combination of sequences in a chimeric polypeptide is typically a sequence that is not found in nature. In some embodiments, a chimeric polypeptide is a polypeptide that includes peptide sequences that are not only found in two or more proteins, but also are mass tag sequences for the two or more proteins. Such mass tag sequences are sequences found in proteins that contain sufficient information (such as sequence information or mass) to permit identification of the proteins from which they are derived. In other words, the identification of the mass tag sequence in a sample permits identifies the protein from which it is derived in the sample.

Cleavage peptide: A peptide generated by proteolytic cleavage of a protein or polypeptide with a protein cleavage agent. Such proteolytic peptides include peptides produced by treatment of a protein with one or more endoproteases such as trypsin, chymotrypsin, endoprotease ArgC, endoprotease aspN, endoprotease gluC, and endoprotease lysC, as well as peptides produced by chemical agents such as cyanogen bromide, formic acid, and thiotrifluoroacetic acid. One or more cleavage peptides from a particular protein may be a mass tag for the protein.

Corresponding: The term “corresponding” is a relative term indicating similarity in position, purpose or structure. “Corresponding peptides” or “corresponding mass tags” refers to either two or more peptides that have the same sequence but different masses or two or more peptides of the same sequence and mass but from different sources. In one embodiment, a mass tag sequence from a target protein and an identical sequence in a disclosed chimeric polypeptide (regardless of whether or not they have the same mass) are described as “corresponding.” In other embodiments, mass spectral signals in a mass spectrum that are due to corresponding peptides of identical structure but differing masses are “corresponding” mass spectral signals. A mass spectral signal due to a particular peptide is also referred to as a signal corresponding to the peptide.

Covalent Modification Reagent: A covalent modification reagent is a reactive molecule that can react with a functional group on another molecule, for example, one or more functional groups (such as —OH, —NH₂, —SH, —CO—, —COOH groups) found on amino acids, peptides, polypeptides and proteins. Covalent modification reagents can be used to prepare peptides that are isotopic analogs of one another. In one embodiment, isotopic analogs of mass tag peptides are prepared by treating one set of peptides with a covalent modification reagent (such as a ²H₂O or H₂¹⁸O that are used to exchange H and ¹⁶O atoms off of the peptides to provide peptides labeled with ²H or ¹⁸O) and not treating another set (although in reality, a peptide sample dissolved in naturally-occurring water will exchange protons and oxygen atoms with the solvent). Treatment with H₂¹⁸O during proteolysis of sample proteins (or the chimeric polypeptide) is one such method of incorporating heavy ¹⁸O atoms into peptides (see, for example, Yao et al., “Proteolytic ¹⁸O Labeling for Comparative Proteomics: Model Studies with Two Serotypes of Adenovirus,” Anal. Chem. 73: 2836-2842, 2001). In this method, enzymatic proteolysis (such as with trypsin) incorporates an oxygen atom from the solvent into the C-terminus of resulting peptides. Thus, either the sample proteins or the chimeric polypeptide can be proteolyzed in heavy (¹⁸O) water and the other in light (¹⁶O) water to provide standard and sample peptides that are isotopic analogs of each other. In another embodiment, sets of peptides are separately treated with two versions of a covalent modification reagent: one version that is isotopically-labeled and one version that is not. For example, one of peptides is reacted with a first covalent modification reagent and another set is treated with a second covalent modification reagent that is an isotopic analog of the first reagent. In other words, after reaction of the two sets with different reagents that have the same structure but different masses, the two sets of modified peptides will have the same structure but different masses and are thus isotopic analogs of each other. An example of this type of labeling scheme is the Isotope-coded Affinity Tag (ICAT) scheme, wherein two versions of reagents that react with specific functional groups on peptides are used to separately treat standard and sample peptides (either separated or part of the chimeric polypeptide or sample proteins, respectively). Examples of ICAT reagents include those described by Gygi et al. (Gygi et al., “Quantitative Analysis of Complex Protein Mixtures Using Isotope-coded Affinity Tags,” Nat. Biotechnol., 17: 994-999, 1999). In the method of Gygi et al., biotinylated iodoacetamide derivatives in a heavy form (deuterated) and in a light form are used to label cysteines of two protein extracts before treatment with a protein cleavage agent. The primary amine group is also a target for introducing an isotopic label, and may be used where peptides of interest do not contain cysteine. For example, the deuterated and non-deuterated forms of N-acetoxysuccinimide or acetate can be used to differentially label the N-terminus and the amino groups of lysines (see, for example, Ji et al., “Strategy for Qualitative and Quantitative Analysis in Proteomics Based on Signature Peptides, J. Chromatogr. B Biomed. Sci. Appl., 745: 187-210, 2000). Cleavable ICAT reagents are commercially available from Applied Biosystems (Foster City, Calif.).

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule.

Expression: The process whereby the genetic information contained in a nucleotide sequence is converted into other cellular components, such as mRNA and protein. Generally, expression of a nucleotide sequence takes place within a cell, but can also take place in a cell-free system.

Genetic lesion: A defect within the genetic material of an organism that affects some function within the organism. Genetic lesions include defects in cellular metabolic pathways. For example, a genetic defect affecting one or more of the genes involved in amino acid biosynthesis is a genetic lesion.

Host cell: A host cell is a cell that is used to express a nucleic acid sequence coding for a chimeric polypeptide. Examples of host cells include microorganisms such as bacteria, protozoans, yeast, viruses and algae, and cultured cells such as cultured human, porcine and murine cell lines.

Internal Standard: An internal standard is a compound that is added in a known amount to a sample prior to sample preparation and/or analysis and serves as a reference for calculating the concentrations of the components of the sample. Isotopically-labeled peptides are particularly useful as internal standards for peptide analysis since the chemical properties of the labeled peptide standards are almost identical to their non-labeled counterparts. Thus, during chemical sample preparation steps (such as chromatography, for example, HPLC) any loss of the non-labeled peptides is reflected in a similar loss of the labeled peptides. Alternatively, the internal standard can be unlabeled when the sample is isotopically-labeled.

Isolated: An “isolated” biological component (such as a nucleic acid, peptide or protein) has been substantially separated, produced apart from, or purified away from other biological components in the cell of the organism in which the component naturally occurs or is transgenically expressed, that is, other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

Isotopic analog: “Isotopic analog” refers to a molecule that differs from another molecule in the relative isotopic abundance of an atom it contains. For example, peptide sequences containing identical sequences of amino acids, but differing in the isotopic abundance of an atom, are isotopic analogs of each other. Similarly, covalent modification reagents that have identical structures but differing isotope content are isotopic analogs, which can be separately reacted with corresponding peptides (identical sequence, different source) to provide covalently modified peptides that are isotopic analogs of one another such as covalently modified sample and standard peptides. The term “isotopic analog” is a relative term that does necessarily not imply that the isotopic analog necessarily contains an isotope that is present in less or greater abundance in nature. For example, a mass tag containing a natural abundance of ¹²C and ¹³C is an isotopic analog of a corresponding mass tag having non-natural abundances of these isotopes, and vice versa.

Isotopically-altered medium: An isotopically-altered medium is a growth medium that is enriched in one or more stable heavy isotopes of an element or elements relative to their natural isotopic abundances. For example, a growth medium including greater amounts of ²H, ¹³C, ¹⁵N and/or ¹⁸O than are found in nature is an isotopically-altered medium. Enrichment of the medium with stable heavy isotopes may be partial (where both heavy and light isotopes of a particular element are present in the medium), or uniform (where substantially only heavy isotopes of a particular element are present, such as greater than 90%, 95%, 98% or 99% of the atoms of an element are the heavy isotope). Stable heavy isotopes may be added to the medium in any form. For example, the isotopes may be added in the form of a simple chemical substance such as ¹⁵NH₃, or may be added in the form of a more complex substance such as an isotopically-altered amino acid (for example, amino acids labeled with ²H, ¹³C, ¹⁵N and/or ¹⁸O, such as deuterium-enriched leucine, serine, and/or tyrosine)

Isotopically-labeled or labeled: “Isotopically-labeled” or “labeled” refer to a molecule that includes one or more stable heavy isotopes in a greater-than-natural abundance. Heavy stable isotopes include, for example, ²H, ¹³C, ¹⁵N, and ¹⁸O.

Mass Spectrometry: Mass spectrometry is a method where a sample is analyzed by generating gas phase ions from the sample, which are then separated according to their mass-to-charge ratio (m/z) and detected. Methods of generating gas phase ions from a sample include electrospray ionization, matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography. In a particular embodiment, separation of a sample by liquid chromatography is followed by MALDI ionization and separation of the resulting ions with high resolution, using one or more stages of mass separation. In another particular embodiment, HPLC separations using reverse phase column (such as a c-4, C-8 or C-18 column) followed by electrospray ionization and mass analysis using a TOF, Q, IT or FT-ICR mass analyzer or some combination thereof.

Mass tag (Mass tag sequence): A mass tag is a peptide (or a set of peptides) having a particular sequence(s) that is (are) uniquely generated from a protein of interest by treatment with a particular protein cleavage agent. Detection of a mass tag for a protein of interest in a sample unambiguously identifies the presence of the protein of interest in a sample treated with the protein cleavage agent, and determination of the concentration or amount of the mass tag in a sample also determines the concentration or amount of the protein of interest in the sample, either directly or after multiplying the concentration of the mass tag by the number of such mass tags generated per protein of interest (for example, where the protein of interest is a multimer of similar or identical sequences). Mass tags may be generated by treating proteins with a protein cleavage agent in vivo, in vitro or in silico. Various methods and algorithms for determining a mass tag for a protein of interest are known, but all have in common that peptide sequences obtained by digestion (actual or theoretical) of a protein of interest with a protein cleavage agent (such as an endoprotease or a model of an endoprotease's cleavage specificity) are compared to peptide sequences obtained by digestion of other known proteins with the same cleavage agent to determine one or more peptide sequences that are uniquely produced from the protein of interest. In other words, a mass tag is a single peptide sequence or a combination of such sequences that is not produced by digestion of any other protein except the protein of interest. Typically, selection of mass tags involves making a comparison of sequences present in protein databases with the sequence of the protein of interest to see if cleavage of the protein of interest provides a unique peptide or peptides in comparison to other known proteins. Various methods that automate the process of identifying mass tags are known. These methods generally share the following sequence of steps. Peptides are generated by digestion of the sample protein using sequence-specific cleavage reagents that allow residues at the carboxyl- or amino-terminus to be considered fixed for the search. For example, the enzyme trypsin that is often used to generate mass tags leaves arginine (R) or lysine (K) at the carboxyl terminus, and the N-termini are expected to be the amino acid following a K or R residue in the protein sequences (except of course for the peptide generated from the N-terminus of the protein, which has a sequence that begins with the N-terminal amino acid of the protein and ends with either a K or R residue). Peptide masses are measured as accurately as possible in a mass spectrometer. An increase in mass accuracy will decrease the number of isobaric peptides (peptides with the same apparent mass) for any given mass in a sequence database and therefore increase the stringency of the search. The proteins in the database are “digested” in silico (i.e. with a computer program) using the rules that apply to the protein cleavage agent used in the experiment to generate a list of theoretical masses that are compared to the set of measured masses. Both protein and DNA sequence databases can be used because the DNA sequences can be translated into protein sequences prior to digestion. An algorithm is then used to compare the set of measured peptide masses against those sets of masses predicted for each protein in the database and to assign a score to each match that ranks the quality of the matches. One or more masses with a high quality score for identification of the protein can be used as a mass tag for the identified protein. An accurate mass tag (AMT) is a single peptide sequence that identifies a protein. As the resolution of the mass spectrometric technique used to measure peptide masses increases, the more likely detection of a single mass signal at a particular mass will unambiguously identify a protein in the presence of peptides from other proteins. FT-ICR-MS is a high resolution mass spectrometric method that can be used to identify AMTs.

As disclosed herein, reference mass tag sequences can be used to identify, detect and/or quantitate corresponding target mass tag sequences.

Nucleotide: A base, such as a pyrimidine, purine, or synthetic analogs thereof, linked to a sugar, plus a phosphate, which forms one monomer in a polynucleotide. A nucleotide sequence refers to the sequence of bases in a polynucleotide.

Oligonucleotide or “oligo”: Multiple nucleotides (that is, molecules comprising a sugar (for example, ribose or deoxyribose) linked to a phosphate group and to an exchangeable organic base, which is either a substituted pyrimidine (Py) (for example, cytosine (C), thymine (T) or uracil (U)) or a substituted purine (Pu) (for example, adenine (A) or guanine (G)). The term “oligonucleotide” as used herein refers to both oligoribonucleotides and oligodeoxyribonucleotides. The term “oligonucleotide” also includes oligonucleosides (that is, an oligonucleotide minus the phosphate) and any other organic base polymer. Oligonucleotides can be obtained from existing nucleic acid sources (for example, genomic or cDNA), but are preferably synthetic (that is, produced by oligonucleotide synthesis).

Peptide/Protein/Polypeptide: All of these terms refer to a polymer of amino acids and/or amino acid analogs that are joined by peptide bonds or peptide bond mimetics. The twenty naturally-occurring amino acids and their single-letter and three-letter designations are as follows:

| Amino Acid | Single-letter Symbol | Three-letter Symbol |
|---|---|---|
| Alanine | A | Ala |
| Cysteine | C | Cys |
| Aspartic Acid | D | Asp |
| Glutamic acid | E | Glu |
| Phenylalanine | F | Phe |
| Glycine | G | Gly |
| Histidine | H | His |
| Isoleucine | I | Ile |
| Lysine | K | Lys |
| Leucine | L | Leu |
| Methionine | M | Met |
| Asparagine | N | Asn |
| Proline | P | Pro |
| Glutamine | Q | Gln |
| Arginine | R | Arg |
| Serine | S | Ser |
| Threonine | T | Thr |
| Valine | V | Val |
| Tryptophan | W | Trp |
| Tyrosine | Y | Tyr |

Predictable mass difference: a predictable mass difference is a difference in the molecular mass of two molecules or ions (such as two peptides, peptide ions) that can be calculated from the molecular formulas and isotopic contents of the two molecules or ions. Although predictable mass differences exist between molecules or ions of differing molecular formulas, they also can exist between two molecules or ions that have the same molecular formula but include different isotopes of their constituent atoms. A predictable mass difference is present between two molecules or ions of the same formula when a known number of atoms of one or more type in one molecule or ion are replaced by lighter or heavier isotopes of those atoms in the other molecule or ion. For example, replacement of a ¹²C atom in a molecule with a ¹³C atom (or vice versa) provides a predictable mass difference of about 1 atomic mass unit (amu), replacement of a ¹⁴N atom with a ¹⁵N atom (or vice versa) provides a predictable mass difference of about 1 amu, and replacement of a ¹H atom with a ²H (or vice versa) provides a predictable mass difference of about 1 amu. Such differences between the masses of particular atoms in two different molecules or ions are summed over all of the atoms in the two molecules or ions to provide a predictable mass difference between the two molecules or ions. Thus, for example, if two molecules have the formula C₆H₆, where one molecule includes 6 ¹³C atoms and the other includes 6 ¹²C atoms, the predictable mass difference between the two molecules is about 6 amu (1 amu difference/carbon atom).

Predictable number of sites: the phrase “a predictable number of sites” refers to an expected number of atoms or groups of atoms in a molecule that will be replaced by atoms or groups of atoms having a predictable mass difference from the atoms or groups of atoms being replaced. For example, if a peptide sequence containing a total of 20 nitrogen atoms is expressed in a host cell grown on a medium that contains ¹⁵NH₃ as the sole nitrogen source, it is expected (and thus predictable) that ¹⁵N atoms will be present in the 20 sites where nitrogen atoms are present in the peptide sequence. If the 20 ¹⁴N atoms of the peptide sequences are replaced with ¹⁵N atoms, a predictable mass difference of about 20 amu will be present between the labeled and unlabeled versions of the peptide (20 times the predictable mass difference per nitrogen atom of about 1 amu). Likewise, where a peptide sequence is expressed in a host cell grown on a medium that contains isotopically labeled leucine, it is expected, and thus predictable, that the isotopically-labeled leucine will be incorporated into the peptide sequence wherever a leucine residue is present in the sequence. Therefore, if the peptide sequence contains 2 leucine residues, the predictable number of sites where the isotopically-labeled leucine residues will be incorporated is two. Incorporation of two L-leucine-5,5,5-d₃ residues (leucines with 3 deuterium atoms instead of hydrogen atoms) provides a predictable mass difference between a peptide sequence incorporating the isotopically-labeled leucines and a peptide sequence with naturally-occurring leucine of about 6 amu (2 leucine sites×3 amu difference in mass per leucine).

Protein cleavage agent: An agent that cleaves a polypeptide or protein into smaller fragments. Protein cleavage agents include biological agents (such as proteolytic enzymes) and chemical protein cleavage agents (such as cyanogen bromide). Typically, but not always, protein cleavage agents cleave peptides and proteins at specific peptide bonds between pairs of particular amino acids. Where specific bonds are cleaved by a protein cleavage agent, the bonds that are cleaved are referred to as “protein cleavage agent sites.” Examples of proteolytic enzymes include endoproteases such as trypsin, chymotrypsin, endoprotease ArgC, endoprotease aspN, endoprotease gluC, and endoprotease lysC. Examples of chemical protein cleavage agents include cyanogen bromide, formic acid, and thiotrifluoroacetic acid. The specific bonds cleaved by an endoprotease or a chemical protein cleavage agents may be more specifically referred to as “endoprotease cleavage sites” and “chemical protein cleavage agent sites,” respectively. Proteins typically contain one or more intrinsic protein cleavage agent sites that are recognized by one or more protein cleavage agents by virtue of the amino acid sequence of the protein.

Proteome: A “proteome” is, in simplest terms, the protein complement expressed by an organism. A “sub-proteome” is a portion or subset of the proteome. The disclosed methods are useful for obtaining quantitative information regarding the proteome of an organism or organisms and sub-proteomes thereof. Exemplary sub-proteomes that may be explored using the disclosed methods include a set of proteins involved in a selected metabolic or signaling pathway (for example, the proteins that mediate glycolysis or lipogenesis, or proteins involved in a protein kinase signal cascade), a set of proteins having a common enzymatic activity (for example, G-protein receptors or protein kinases), or the proteins from a particular location in an organism or cell. For example, preparations of organelles, ribosomes, cell membranes, nuclear membranes can be analyzed using the provided methods.

Proteomics: The term proteomics refers the study of the composition of the protein complement of an organism or organisms. “Quantitative proteomics” refers to the study of the relative or absolute amounts or concentrations of the proteins expressed by an organism or organisms, in one or more states. For example, since organisms respond to changes in their environment by producing different proteins, the one or more states may be environmental or pathological states, such as states due to exposure to a toxin or drug or the presence of a cancer in the organism.

Separable by a protein cleavage agent: the phrase “separable by a protein cleavage agent” refers to portions of a peptide sequence that can be cleaved apart to form separate sub-sequences of the peptide by treatment with a protein cleavage agent. The portions of a peptide sequence that are separable by a protein cleavage agent typically are separated by specific bonds between particular amino acids that are recognized and cleaved by a particular protein cleavage agent. For example, four peptide sequences that each include a lysine (K) residue at their C-terminus, and are joined end-to-end in a single polypeptide, are separable by the protein cleavage agent trypsin, which recognizes lysine residues and cleaves a polypeptide sequence on the carboxyl side of the lysine residue to yield the original four peptide sequences.

Standard: A standard is a substance or solution of a substance of known amount, purity or concentration. A standard can be compared (such as by spectrometric, chromatographic, or spectrophotometric analysis) to an unknown sample (of the same or similar substance) to determine the presence of the substance in the sample and/or determine the amount, purity or concentration of the unknown sample.

Synthetic: A synthetic nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

Target protein: A protein or fragment of a protein for which identification or quantification is desired. Specific examples of pre-selected target proteins are provided throughout the specification and include enzymes of particular metabolic or signaling pathways, and proteins of various classes, subclasses or sub-subclasses.

Multiple target proteins can be pre-selected for identification and/or quantification. The target proteins can be grouped based on a shared property, such as one or more common properties, that allow the group to be distinguished from other proteins. For example, the target proteins can be grouped based on a shared structure, function, chemical, or other property, such as a relationship in a particular class of enzymes, a class of protein involved in a known biochemical pathway, a relationship in a particular transport protein complex, a relationship in a particular membrane-associated protein complex, a class of protein involved in a known transcriptional or translational pathway, and the like.

Uniformly labeled: As applied to a growth medium, this term refers to a growth medium wherein substantially all atoms (such as greater than 90%, 95%, 98% or 99% of all atoms) of a particular element present in the medium are present in the form of a particular isotope of the element. A uniformly-labeled growth medium provides a particular type of atom substantially in the form of a single isotope of the atom. For example, a medium that provides nitrogen in the form of ¹⁵NO₃— as the sole nitrogen source for host cells grown on the medium is an uniformly-labeled medium.

Uniquely associated: As applied to a peptide sequence derived from a larger peptide sequence (such as a protein sequence), a peptide sequence or a combination of peptide sequences that is “uniquely associated” with the larger peptide sequence is a sequence or a combination of sequences that is not present in any other larger peptide sequences of a sample besides the one from which it is derived. Thus, identification of the peptide sequence that is “uniquely associated” with the larger peptide sequence in a sample identifies the larger peptide sequence in the sample. For example, a peptide that is obtained from a target protein by digestion of the target protein with an endoprotease (such as trypsin) is “uniquely associated” with the target protein if detection of the peptide in the presence of other peptides that are obtained from other proteins in the sample by digestion with the same endoprotease is sufficient to unambiguously identify the target protein in the sample. In other words, a peptide that is uniquely associated with a target protein contains enough sequence (and mass) information to discriminate between the target protein and other proteins in the sample.

III. Overview

High-throughput quantitative analysis of protein samples by mass spectrometry is facilitated by the disclosed methods for producing peptide standards. The peptide standards are produced by expressing a chimeric polypeptide in a host cell where the chimeric polypeptide includes peptide standard sequences such as mass tags for two or more proteins that may be present in a sample. A mass tag is a peptide sequence or set of sequences that can be used to identify particular proteins in the sample, for example, by detecting a mass spectral signal at a mass-to-charge ratio corresponding to the characteristic mass of the peptide. Within the chimeric polypeptide sequence, the peptide standard sequences are separated by protein cleavage sites that are specifically recognized by a protein cleavage agent such as an endoprotease. Upon treatment of the chimeric polypeptide with a protein cleavage agent, the constituent peptide standard sequences are liberated for use. The chimeric polypeptide can be labeled with heavy stable isotopes (such as ¹⁵N) by growing the host cell grown in a medium including the isotope (for example, in the form of an isotopically-labeled amino acid that is incorporated into the chimeric polypeptide during host cell growth), or unlabeled.

The disclosed chimeric polypeptides and the peptide standard sequences that they include can be used as internal standards for absolute quantitation of sample proteins. In one embodiment, the chimeric polypeptide is expressed in a host cell, isolated, quantified, and added directly to a sample in a known amount. The sample containing the known amount of the peptide standards is then treated with a protein cleavage agent (such as trypsin) and analyzed by mass spectrometry. Alternatively, the chimeric polypeptide and the sample are separately treated with a protein cleavage agent. In this instance, the peptide standards derived from the chimeric polypeptide are added to the separately treated sample in a known amount, and then the sample is analyzed by mass spectrometry. At least one of either the sample proteins or the chimeric polypeptide can be isotopically-labeled. Isotopic labeling of the chimeric polypeptide or sample proteins can be accomplished by expression in an isotopically-altered medium, or by separate covalent modification of the chimeric polypeptide (or its separated standard peptides) and the sample proteins (or their separated sample peptides) with different versions of a covalent modification reagent that differ in mass, but typically do not differ in molecular formula. In either case, the sample peptides derived from the sample proteins and the standard peptides derived from the chimeric polypeptide will have different masses (that is, they are isotopic analogs) so that separate mass spectral signals for each will be evident in a mass spectrum. The ratio of mass spectral signals for the sample peptides and for the standard peptides reflects the relative amounts of each in a sample. If the absolute amount (concentration) of the standard peptides is known, the absolute amount (concentration) of the sample peptides can be calculated using the ratio derived from the mass spectral signals. Since the amount of sample peptide is typically equal to the amount of sample protein from which it is derived, quantitation of the sample peptides following digestion of the sample proteins is typically equivalent to quantitation of the sample proteins. In other words the amount or concentration of sample proteins is typically equal to the amount or concentration of the peptides measured by MS. Even if the amounts or concentrations of the sample peptides are not the same as the amounts or concentrations of the sample proteins, they will be related by a known ratio. For example, for a dimeric protein with two identical polypeptide chains the amount or concentration of the protein will be ½ of the measured amount or concentration of a unique peptide liberated from each of the polypeptide chains of the dimeric protein by cleavage with a protein cleavage agent.

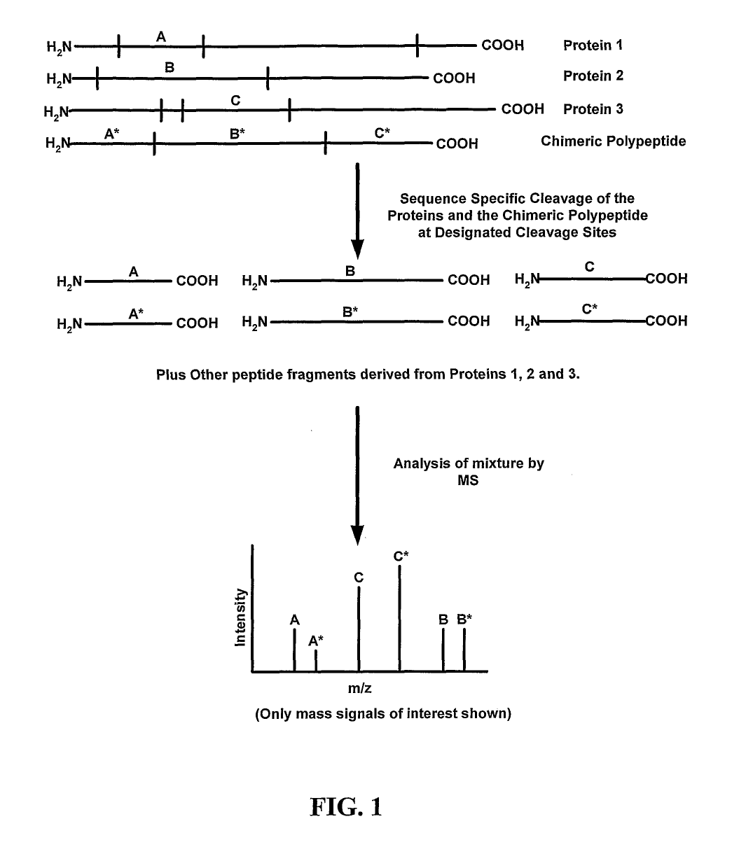

An embodiment of a method to quantify sample proteins using a disclosed chimeric polypeptide and mass spectrometric measurement is outlined in FIG. 1. In this embodiment, the protein sample is assumed to consist of three proteins (1, 2 and 3), the amounts of which are to be determined. The three proteins could, for example, first have been separated from other sample proteins by a chromatographic technique such as high-pressure liquid chromatography. As shown in FIG. 1, each of the three sample proteins includes a mass tag sequence (an accurate mass tag in this case). The mass tag for protein 1 is designated A, the mass tag for protein 2 is designated B, and the mass tag for protein 3 is designated C. These mass tag sequences are generated from the three sample proteins by cleavage of the proteins at specific sites with a particular protein cleavage agent (for example, trypsin). The cleavage sites are denoted by vertical bars distributed along the sequences of Proteins 1, 2 and 3. A known amount of a chimeric polypeptide is added to the sample proteins to form a mixture. The chimeric polypeptide includes isotopic analogs of the mass tags from proteins 1, 2 and 3 that also are separated by cleavage sites recognized by the same protein cleavage agent (for example, trypsin). The isotopic analogs of sample protein mass tag sequences A, B and C that are included in the chimeric polypeptide are denoted A*, B* and C*, respectively. In this embodiment, proteins 1, 2, and 3 are not labeled and the chimeric polypeptide is labeled with heavy stable isotopes. In this case, the heavy stable isotopes incorporated into the chimeric polypeptide positively offset the masses of A*, B* and C* from the masses of A, B and C.

As shown in FIG. 1, simultaneous treatment of the sample proteins 1, 2, and 3 and the added chimeric polypeptide with the same protein cleavage agent (trypsin) yields 6 peptides of interest (A, B, C, A*, B*, and C*) along with other peptides derived from the sample proteins. The mass tags from the proteins and their isotopic analogs derived from the chimeric polypeptide have identical sequences, but different masses. Analysis of the mixture of digested peptides yields a mass spectrum such as the one shown at the bottom of FIG. 1, which only shows the six peptides of interest as separated peaks at different mass-to-charge ratios (m/z ratios). The mass offset of each mass tag/isotopic analog pair (A/A*, B/B* and C/C*) can be predicted based on the number and type of isotopic substitutions that are present in the isotopically-labeled peptides derived from the chimeric polypeptide. For example, if isotopically-labeled amino acids are incorporated into the chimeric polypeptide, the number of such amino acids in the isotopic analogs multiplied by the difference in mass between the unlabeled and labeled versions of the amino acid is equal to the mass offset observed between each mass tag/isotopic analog pair. In FIG. 1, the mass spectrum shows that the mass offsets between A and A* and between B and B* are equal, whereas the mass offset between C and C* is twice that of the others. Such a situation could arise where two isotopically-labeled amino acids are incorporated into C*, whereas only one isotopically-labeled amino acid is incorporated into each of A* and B*.

Once a mass tag/isotopic analog pair is identified, the signal intensities for the mass tag and its isotopic analog are used to generate a ratio that reflects the relative amounts of the mass tag peptide (and the protein from which it is derived) and the isotopic analog peptide. For example, the ratio of the signal intensities of A and A* in FIG. 1 indicates that the mass tag peptide A (and protein 1 of the sample) is present at a concentration (amount) that is lower than the known amount of A* that was added to the sample as part of the chimeric polypeptide. Assuming A and A* provide signals in a ratio of 2:1, the absolute amount of A (and protein 1) in the sample is equal to the known amount of the chimeric polypeptide (which is presumed to be equal to the amount of its constituent mass tags) multiplied by the ratio. For example, if the amount of the chimeric polypeptide that was added to the sample was 1 nmole, the amount of protein 1 in the sample would be calculated to be 2 nmoles because the amount of the chimeric polypeptide is equal to the amount of A*, the amount of A (from protein 1) is twice the amount of A* (as determined from the mass spectrum), and the amount of protein 1 is equal to the amount of A. The concentrations of B (protein 2) and C (protein 3) can be calculated in the same manner based on the ratios of the mass signal intensities for B and B*, and C and C*, respectively.

In some embodiments, mass tag sequences that are included in the sequence of the disclosed chimeric polypeptides are selected to provide peptide standards for quantitative analysis of two or more proteins of interest. In particular embodiments, the mass tags that are combined in the chimeric polypeptide are mass tags for proteins that are typically present in biological samples in similar concentrations, such as within one to two orders of magnitude in concentration. For example, mass tags for high abundance proteins such as structural proteins may be combined in a chimeric polypeptide, or mass tags for low abundance proteins such as regulatory or signaling proteins may be combined in a chimeric polypeptide. Multiple chimeric polypeptides that each are combinations of mass tags for different sets of proteins of similar abundances may be used to provide standards for proteins that span a wide range of abundances. Alternatively, mass tags for different proteins spanning several ranges of abundances are combined in a single chimeric polypeptide.

Since the sequence(s) of the mass tag(s) for a particular protein depends upon the protein cleavage agent used to generate the mass tag(s), the disclosed chimeric polypeptides will typically include mass tags that are generated by a single protein cleavage agent. However, in some embodiments, mass tags that are generated by multiple protein cleavage agents may be combined in the chimeric polypeptides. In addition to mass tag sequences, the chimeric polypeptides may include other sequences such as spacer amino acid sequences between one or more mass tag sequences, one or more affinity purification sequences and one or more sequences rich in UV-absorbing amino acids such as tryptophan or tyrosine.

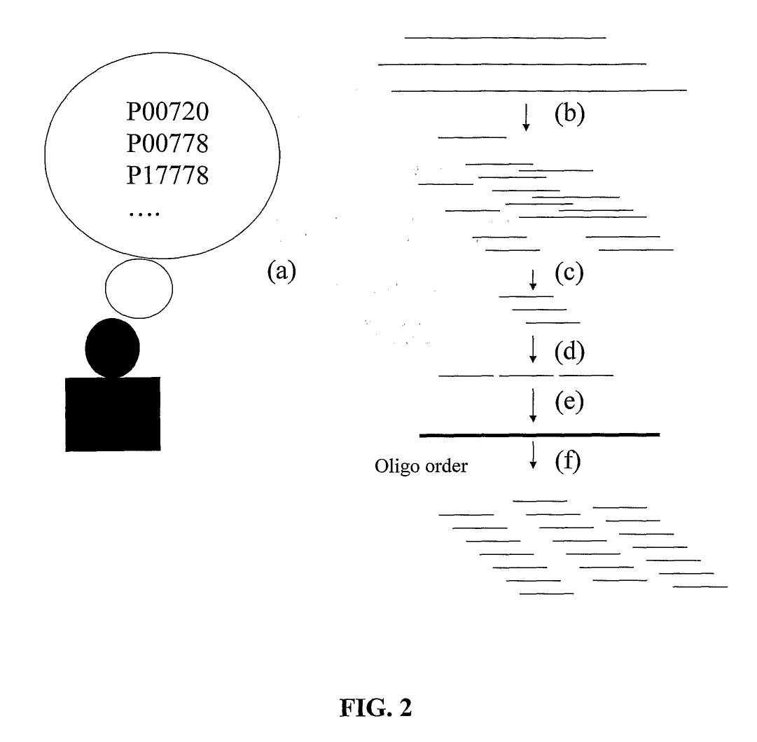

An exemplary method for designing a chimeric polypeptide according to the disclosure is presented in FIG. 2. With reference to FIG. 2, a set of proteins for which quantitative information is desired is selected (Step a). In step (b) of FIG. 2, the sequences of the selected proteins are cleaved according to a computer model of treatment of the sequences with the endoprotease trypsin (an in silico digestion of the proteins with trypsin). At least one tryptic peptide sequence is selected (Step c) for each of the proteins in the set of proteins. The selected peptide sequences are then combined (Step d) to provide a larger chimeric polypeptide sequence. Additional sequences, such as spacer sequences and sequences that can aid later purification of the chimeric polypeptide may be added to the sequence at this point. The sequence is then back-translated (Step e, performed, for example, with a computer program that uses the genetic code) to a desired nucleic acid sequence coding for the chimeric polypeptide, and an optimal set of oligonucleotide primers is determined (Step f, performed, for example, with a computer program) that can be used to generate the desired nucleic acid sequence by, for example, assembly PCR

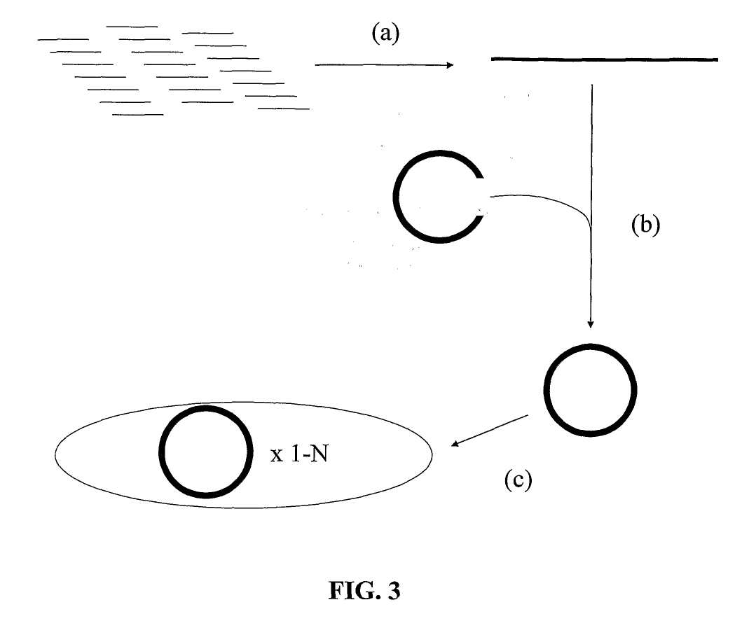

The synthetic nucleic acid sequence encoding the chimeric polypeptide is then inserted into an expression vector using recombinant DNA techniques, and the vector is introduced into an appropriate host cell, which can be grown on an isotopically-altered medium. FIG. 3 shows an exemplary embodiment of the process of synthesizing the desired nucleic acid sequence coding for the desired chimeric polypeptide, and introducing it into an expression vector. With reference to FIG. 3, a set of synthetic oligonucleotides are used as primers for producing (Step a) the nucleic acid sequence coding for the chimeric polypeptide by assembly PCR. The assembled nucleic acid sequence is then inserted (Step b) into an appropriate expression vector. A host cell is then transformed (Step c) with the expression vector. The expression vector can then be stored or propagated in the host cell, or used to transform another type of host cell.

In one embodiment, an isotopically-labeled chimeric polypeptide is produced by growing a transformed host cell on an isotopically-altered medium. The host cell may be grown, for example, on a medium that is uniformly labeled with an isotope (such as a medium containing 15N as the sole nitrogen source) or a medium that includes one or more particular isotopically-labeled amino acids (such as ¹⁵N-labeled arginine or lysine). Alternatively, where the sample is isotopically-labeled, the host cell may be grown on a non-labeled medium.

As mentioned above, in some embodiments the chimeric polypeptide comprises mass tags for two or more different proteins that are separated by one or more protein cleavage agent sites (for example, 5 or more, 10 or more, 15 or more, or 25 or more different mass tags separated by protein cleavage agent sites). The multiple protein cleavage agent sites may be the same cleavage site (so that a single protein cleavage agent such as a particular endoprotease recognizes and cleaves the chimeric polypeptide at the sites) or different cleavage sites (that are recognized by multiple different protein cleavage agents such as multiple different endoproteases).

Once expressed by the host cell, the chimeric polypeptide is isolated from the host cell. In one embodiment, the expressed chimeric polypeptide forms inclusion bodies. The inclusion bodies are isolated from the host cell and treated to further isolate the expressed chimeric polypeptide therefrom. In addition, the isolated chimeric polypeptide also may be treated with a protein cleavage agent that recognizes the internal protein cleavage agent sites to the mass tag standards from the chimeric polypeptide. The protein cleavage agent sites may be endoprotease cleavage sites (such as trypsin cleavage sites) or chemical protein cleavage agent sites (such as cyanogens bromide cleavage sites).

The mass tags that are included in the chimeric polypeptide may, for example, be for two or more different proteins of interest, such as proteins that are present in biological samples at substantially similar concentrations (such as present in concentrations spanning one to two orders of magnitude), that are different enzymes of the same metabolic or signaling pathway (for example, enzymes of the citric acid cycle), that are proteins of the same class (such as transferases), subclass (such as transferases that transfer sulfur-containing groups) or sub-subclass (such as acetyl-CoA transferases).

Additional sequences such as spacer amino acids, or more particularly, affinity sequences (such as a poly-histidine sequence) or sequences including UV-absorbing amino acids (such as tryptophan and tyrosine) may be included in the chimeric polypeptides. Affinity sequences assist in isolation and purification of the chimeric polypeptide from the host cell, and in particular, they can assist in isolation of the chimeric polypeptide from inclusion bodies. Sequences including UV-absorbing amino acids can assist in quantifying the chimeric polypeptide isolated from a host cell so that it (or the peptide sequences it includes) can be added to a sample in known amounts.

In some embodiments, the disclosed methods of quantitative proteomics include mixing a known amount of an isotopically-labeled chimeric polypeptide with an unlabeled protein sample and treating the mixture with a protein cleavage agent (such as trypsin) under conditions that permit cleavage of the chimeric polypeptide to occur at the cleavage sites between the mass tags included in the chimeric polypeptide and cleavage of the sample proteins into corresponding mass tag peptide sequences. As a result of treatment with the protein cleavage agent, mass tags are released from the chimeric polypeptide and from the proteins in the sample. Because cleavage of substantially identical amino acid sequences of the chimeric polypeptide and the target protein take place with the same cleavage agent, the sequences of the resulting mass tags are identical (or substantially identical) but differ in mass because of their relative isotopic contents. The isotopically-labeled mass tags liberated from the chimeric polypeptide serve as internal standards for quantitation of the proteins in the sample by spectrometric techniques such as mass spectrometry. Alternatively, the chimeric polypeptide and the protein sample may be treated separately with the same protein cleavage agent, and then mixed. Labeled and unlabeled mass tags having the same sequence(s) of amino acids are referred to as “corresponding” with each other. In an alternative embodiment, the chimeric polypeptide is unlabeled and the sample proteins are isotopically-labeled. In yet another embodiment, the sample proteins and the chimeric polypeptide are separately treated with two versions of a covalent modification reagent (one isotopically-labeled, another not) to provide (after cleavage) covalently modified peptides that are isotopic analogs of each other that are distinctly detectable by mass spectrometry. It is also possible to first separately cleave the sample proteins and the chimeric polypeptide into their constituent mass tag sequences and then treat each with a different version of a covalent modification reagent.

One example of a method for high-throughput quantitative mass spectrometric analysis of a protein sample using the disclosed chimeric polypeptides includes adding a known amount of an isotopically-labeled chimeric polypeptide to the protein sample to provide a combined sample. In this example, the chimeric polypeptide is a combination of mass tags for two or more different proteins that may be present in the sample (such as 5 or more, 10 or more, 15 or more or 25 or more different proteins) that are separated by one or more cleavage sites recognized by one or more protein cleavage agents (for example, sites recognized by endoprotease or chemical protein cleavage agents, such as trypsin cleavage sites or cyanogen bromide sites, respectively). The combined sample is treated with a protein cleavage agent that cleaves both the chimeric polypeptide and the proteins in the sample at one or more intrinsic protein cleavage agent sites recognized by the protein cleavage agent. By treating the sample proteins and the chimeric polypeptide together in the combined sample with the same protein cleavage agent, peptides of similar sequence, but different masses, are produced. Following treatment with the protein cleavage agent, the combined sample is analyzed by mass spectrometry (such as ESI, MALDI, SELDI or FT-ICR mass spectrometry) to provide a mass spectrum. In the mass spectrum, pairs of signals corresponding to pairs of corresponding peptides of the same sequence but different mass can be identified, where the signal at the higher mass-to-charge ratio is due to the peptide from the chimeric polypeptide that was added as an internal standard. The ratio of the signals in each pair, combined with the known amount of the chimeric polypeptide, can then be used to calculate an absolute amount of the sample proteins as described above. In an alternative embodiment, the sample is isotopically-labeled, the chimeric polypeptide is unlabeled, and the signal at the lower mass-to-charge ratio in each pair of corresponding signals in the mass spectrum is due to the chimeric polypeptide added as an internal standard.

Alternatively, a method for high-throughput quantitative mass spectrometric analysis of a protein sample is provided that includes digesting an isotopically-labeled chimeric polypeptide that is a combination of mass tags for two or more different proteins separated by one or more protein cleavage agent sites with a protein cleavage agent to release labeled mass tags. A protein sample also is separately digested with the same protein cleavage agent used to treat the isotopically-labeled chimeric polypeptide to generate corresponding peptide sequences from the sample. A known amount of the labeled mass tags from the isotopically-labeled chimeric polypeptide is then added to the digested protein sample as an internal standard and the combined sample is analyzed by mass spectrometry to provide a mass spectrum. In yet another alternative embodiment, a known amount of an unlabeled mass tag obtained from a chimeric polypeptide by digestion is added as an internal standard to a digested, isotopically-labeled protein sample to provide a combined sample. In either case, spectrometric analysis of the combined sample provides signals for the standard and sample peptides that can be compared and used to calculate absolute concentrations of proteins in the sample.

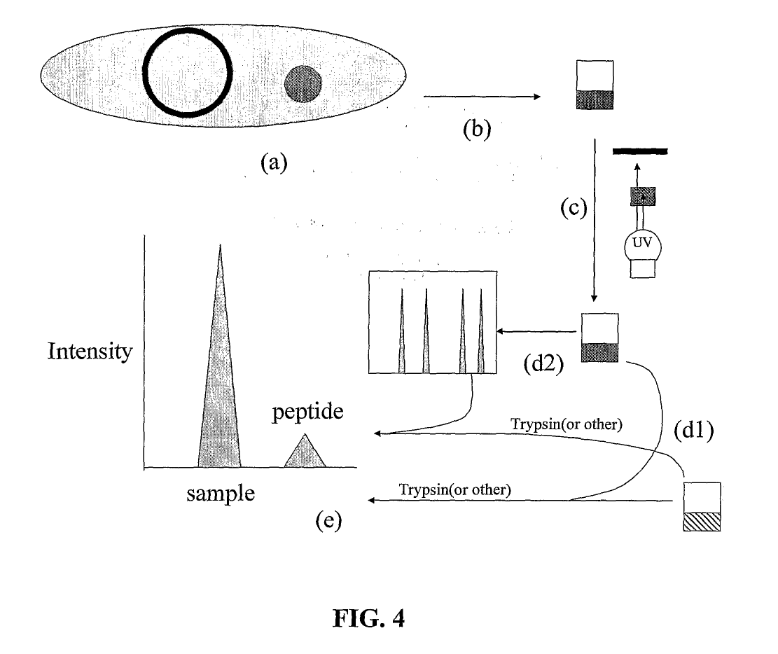

A particular embodiment of the use of the disclosed chimeric polypeptides for quantitative proteomics is shown in FIG. 4. In this embodiment, the host cell expressing the chimeric polypeptide is grown (Step a) in an isotopically-altered medium to provide inclusion bodies that include the desired isotopically-labeled chimeric polypeptide (and the desired isotopically-labeled peptide standards the chimeric polypeptide includes). Inclusion bodies including the desired chimeric polypeptide are then isolated (Step b), for example, by lysing the host cell and purifying the chimeric polypeptide using affinity chromatography (such as immobilized metal affinity chromatography) and/or another isolation methods (such as size-exclusion chromatography, reverse-phase chromatography or ion exchange chromatography) under denaturing conditions (such as 5M guanidinium chloride). The isolated and purified chimeric polypeptide can then be quantitated (Step c), for example, using UV-Vis spectrophotometry. The quantitated chimeric polypeptide is added in a known amount to an uncleaved experimental sample prior to digestion with a protein cleavage agent (Step d1), or the chimeric polypeptide is digested, the peptide standards are separated by chromatography (Step d2) and added to the sample in known amounts after the sample is digested. Samples containing the peptide standards liberated from the chimeric polypeptide are analyzed (Step e) using mass spectrometry.

Once a mass spectrum is provided by any of the methods discussed above, one or more different proteins in the protein sample may be identified based on the presence of a mass signal at a mass-to-charge ratio that is characteristic of an unlabeled (or labeled) mass tag for the one or more different proteins. A corresponding labeled (or unlabeled) mass tag sequence from the chimeric polypeptide also may be identified in the mass spectrum, for example, based on an expected mass-shift caused by the “heavy” isotopes in the labeled mass tag (a predictable mass difference). The absolute amount or concentration of sample proteins may then be calculated using the known amount of the chimeric polypeptide added to the protein sample and the ratio of the intensities of the mass signals for the unlabeled and the corresponding labeled mass tags. The known amount of the chimeric polypeptide (or the labeled mass tags it contains) may be determined, for example, by UV-Vis spectrophotometry or by NMR (see, for example, Cavaluzzi, et al., Analytical Biochemistry, 308: 373-380, 2002).

While simple mixtures of proteins can be examined using the disclosed methods, one advantage of the methods is that they enable simultaneous analysis and quantitation of many different proteins. For example, the methods may be used to simultaneously quantify at least 10, 20, 30 or 50 such constituent proteins of a sample, such as at least 100, 500 or 1000 such constituent proteins of a sample. The advantage arises from the disclosed methods because each chimeric polypeptide can include peptide standards for a large number of proteins. Addition of a chimeric polypeptide (or the peptides it contains) to a sample provides internal standards that can be used to quantify a large number of proteins in the sample from a single mass spectrum.

In one embodiment, a method is provided for making standards for quantitative proteomics by providing a host cell, expressing in the host cell a chimeric polypeptide that comprises different mass tag sequences for two or more different target proteins that are separable by a protein cleavage agent at one or more protein cleavage sites, and isolating the chimeric polypeptide from the host cell.

In another embodiment, the method further involves including an isotope for isotopic labeling in the medium. In this embodiment, expression of the chimeric polypeptide in the medium leads to expression of the chimeric polypeptide with the isotope of the medium incorporated into the mass tag sequences. The mass tag sequences of the chimeric polypeptide are expressed as isotopic analogs of the mass tag sequences of the target proteins, and are detectable as distinct from the mass tag sequences of the target proteins by mass spectrometry. For example, the chimeric polypeptide can be expressed in a medium that is isotopically-altered where the target proteins are not isotopically-labeled, or the chimeric polypeptide can be expressed in a medium that is not isotopically-altered where the target proteins are isotopically-labeled. The chimeric polypeptide is isolated from the host cell to provide a material that can be added to a protein sample in known amounts as an internal standard. Once isolated, the chimeric polypeptide can be cleaved with a protein cleavage agent that separates the chimeric polypeptide into the isotopic analogs of corresponding mass tag sequences of the target proteins, which has been cleaved with the same cleavage agent. Such isolated chimeric polypeptides and their cleaved peptides are included in the disclosure.

In yet another embodiment, the method further includes reacting the mass tag sequences of the chimeric polypeptide with a covalent modification reagent, the covalent modification reagent including an isotope such that the reacted mass tag sequences of the chimeric polypeptide are isotopic analogs of mass tag sequences of the target proteins that have been reacted with a corresponding covalent modification reagent. Such pairs of differentially modified peptides are detectable as distinct by mass spectrometry.

Treatment of the isolated chimeric polypeptide with the protein cleavage agent cleaves the chimeric peptide at the protein cleavage sites to provide separated isotopic analogs of the mass tag sequences of the target proteins. The protein cleavage agents can be enzymatic or chemical cleavage sites recognized by enzymatic or chemical protein cleavage agents, respectively. In some embodiments, the cleaved mass tag sequences of the chimeric polypeptide have identical amino acid sequences as corresponding mass tags from the target proteins. In others, treating the isolated chimeric polypeptide with a protein cleavage agent that recognizes the protein cleavage sites and cleaves the mass tag sequences from the chimeric polypeptide forms separated isotopically-labeled mass tag sequences.

In particular embodiments, the isotope for isotopic labeling in the medium is a stable heavy isotope that is present in the medium in greater abundance relative to its natural isotopic abundance. Incorporation of the isotope into the chimeric polypeptide provides a chimeric polypeptide with mass tag sequences that are detectable as distinct from the mass tag sequences of the target protein by mass spectrometry. In other particular embodiments, the medium does not include an isotope for isotopic labeling. Instead, the target proteins are isotopically labeled, and the isotopic analogs of the mass tag sequences of the target proteins that are included in the chimeric polypeptide are unlabeled mass tag sequences of the target proteins. The isotopic analogs provided in the chimeric polypeptide, albeit unlabeled, are detectable as distinct from isotopically-labeled mass tag sequences of the target proteins by mass spectrometry.

Typically, the mass tag sequences of the chimeric polypeptide and the mass tag sequences of the target proteins are detectable as distinct based on a predictable mass difference between them. The predictable mass difference can be determined by selecting an isotope that is incorporated into a predictable number of sites in the chimeric polypeptide or the target proteins, which aids in location of the mass spectral signals of the pair of corresponding labeled and unlabeled mass tag sequences.

In other particular embodiments, the chimeric polypeptide consists essentially of the mass tags for the two or more different target proteins and an affinity sequence or a sequence that comprises UV-absorbing amino acids. In still others, the chimeric polypeptide comprises at least 10 different mass tags for ten or more different target proteins.

In still other particular embodiments, the method further includes determining a concentration of the chimeric polypeptide, so that the quantitated chimeric peptide can be added to a biological sample in a predetermined amount for quantitation of the two or more target proteins in the biological sample when the target proteins are treated with the protein cleavage agent and analyzed by mass spectrometry. The method can also further include identifying target proteins in the sample because identification of a mass tag sequence peptide for a target protein in a sample serves to identify the target protein.

In still further particular embodiments, the target proteins share a common property.

In another embodiment, a method is provided for high-throughput quantitative mass spectrometric analysis of a protein sample. The method includes cleaving a known amount of chimeric polypeptide with a protein cleavage agent that cleaves the chimeric polypeptide to provide multiple different mass tag sequences having an identical amino acid sequence as corresponding mass tag sequences obtained by cleavage of target proteins with the same protein cleavage agent that cleaves the chimeric polypeptide. Cleavage of the known amount of the chimeric polypeptide provides a known amount of each of the multiple different mass tag sequences comprising the chimeric polypeptide. Mass spectrometry is performed on a sample including the cleaved mass tag sequences from the chimeric polypeptide and from the target proteins. Mass spectrometry is used to measure masses of the mass tag sequences from the chimeric polypeptide and from the target proteins and to predict quantities of the target proteins that are present in the sample. The quantities are calculated using the ratios of mass spectral signals for the known amounts of the mass tag sequences cleaved from the chimeric polypeptide to mass spectral signals for the corresponding mass tag sequences cleaved from the target proteins. Identities of the target proteins may also be determined by comparing the mass of one or more mass tag sequences cleaved from the target protein by the protein cleavage agent with a database comprising masses of peptides generated by digestion of known peptides or proteins using the protein cleavage agent. The target proteins and the chimeric polypeptide can be cleaved into their mass tag sequences after mixing them together, or separately treated with the protein cleavage agent and then combined. Furthermore target proteins and the chimeric polypeptide can be separately treated with different versions of a covalent modification reagent, or the peptides released from the target proteins and the chimeric polypeptide can be separately treated with different versions of a covalent modification reagent. Another possibility is to treat either the target proteins or the chimeric polypeptide with a first version of a covalent modification reagent and treat the peptides liberated from the other with a second version of the covalent modification reagent (of different mass).

In yet another embodiment, a method is provided for high-throughput quantitative mass spectrometric analysis of a protein sample. The method includes providing a chimeric polypeptide that comprises different mass tag sequences for two or more different target proteins that correspond to and are identified by the different mass tag sequences. Each mass tag sequence of the chimeric polypeptide includes an isotopic analog of a corresponding mass tag sequence of its target protein that is distinctly detectable by mass spectrometry from the corresponding mass tag sequence of its target protein. As before, the mass tag sequences of the chimeric polypeptide are separable from each other by a protein cleavage agent at one or more protein cleavage sites. The chimeric polypeptide is expressed with an isotope incorporated into its mass tag sequences. A sample is provided of a known amount of the mass tag sequences of the chimeric polypeptide and unknown amounts of the corresponding mass tag sequences of the target proteins. The corresponding mass tag sequences of the target proteins are obtained by cleavage of the chimeric polypeptide and the target proteins with the same protein cleavage agent used to produce the mass tag sequences from the chimeric polypeptide. Mass spectrometry (such as ESI, MALDI or SELDI based mass spectrometry) is performed on the sample to measure masses of the mass tag sequences of the chimeric polypeptide and of the target proteins that are present in the sample and to predict quantities of the target proteins in the sample by comparing the known amounts of the mass tag sequences from the chimeric polypeptide to the unknown amounts of the mass tag sequences of the target proteins. This is possible since the unknown amounts of the mass tag sequences of the target proteins are equal to unknown amounts of the target proteins. In some particular embodiments, predicting quantities of the target proteins also includes determining the known amount of the chimeric polypeptide by UV-Vis spectrophotometry.

The sample may be provided by mixing the chimeric polypeptide and the target proteins, and then cleaving the mixed chimeric polypeptide and target proteins with the protein cleavage agent. Alternatively, the sample may be provided by cleaving the chimeric polypeptide with the protein cleavage agent to provide the mass tag sequences of the chimeric polypeptide, separately cleaving the target proteins with the same protein cleavage agent to produce the corresponding mass tag sequences of the target proteins, and mixing the mass tag sequences of the chimeric polypeptide and the target proteins. Typically the protein cleavage sites are cleaved by a protein cleavage agent that cleaves the chimeric peptide to provide mass tag sequences having identical amino acid sequences as corresponding mass tag sequences obtained by cleavage of the target proteins with the same cleavage agent. Examples of a suitable protein cleavage agent include an endoprotease such as trypsin (which cleaves at trypsin cleavage sites) or a chemical cleavage agent such as cyanogens bromide.

The chimeric polypeptide or the target proteins can be isotopically labeled. In particular embodiments, the mass tag sequences of the chimeric polypeptide are isotopically-altered, for example with a stable heavy isotope, such that the mass tag sequences of the chimeric polypeptide are detectable by mass spectrometry as distinct from the corresponding mass tag sequences of the target proteins. In other particular embodiments, the target proteins are isotopically-altered with an isotope such that the mass tag sequences of the target proteins are detectable by mass spectrometry as distinct from the mass tag sequences of the chimeric polypeptide. In either case, each mass tag sequence of the chimeric polypeptide has a mass that differs from its corresponding mass tag sequence of its target protein by a predictable mass difference. Such predictable mass differences may be determined by an isotope that is incorporated into a predictable number of sites in the isotopic analog. Isotopic-labeling of the chimeric polypeptide or the target proteins can be accomplished with a heavy stable isotope such as ¹⁵N. In more particular embodiments, isotopic-labeling is accomplished with an isotopically-altered amino acid.

Comparing the known amounts of the mass tag sequences of the chimeric polypeptide to the unknown amounts of the corresponding mass tag sequences of the target proteins to predict quantities of the target proteins can include determining ratios of mass spectral signals for the known amounts of the mass tag sequences of the chimeric polypeptides and for the unknown amounts of corresponding mass tag sequences of the target proteins. The mass tag sequences of the target proteins are peptides that are uniquely associated with particular proteins of which the peptides are fragments. As such, it is also possible to identify one or more target proteins in the protein sample based on the presence of a mass signal in a mass spectrum that appears at a mass-to-charge ratio that is characteristic of a particular protein.

In a particular embodiment, a method is provided for high-throughput quantitative mass spectrometric analysis of a protein sample that includes treating an isotopically-labeled chimeric polypeptide including mass tag sequences for two or more different target proteins that are separated by one or more protein cleavage agent sites and separateable with a protein cleavage agent to release labeled mass tag sequences for the two or more different target proteins from the isotopically-labeled chimeric polypeptide. The protein sample is treated with the same protein cleavage agent used to treat the isotopically-labeled chimeric polypeptide. Treatment of the protein sample in this manner provides a digested protein sample that includes the mass tag sequences of the target proteins. A known amount of the labeled mass tag sequences for the two or more different target proteins from the chimeric polypeptide is added to the digested protein sample to provide a combined sample, which is analyzed by mass spectrometry to provide a mass spectrum.

In an alternative particular embodiment, a method is provided for high-throughput quantitative mass spectrometric analysis of a protein sample. In this embodiment, a known amount of a chimeric polypeptide is added to the protein sample to provide a combined sample, and the combined sample is treated with a protein cleavage agent. The chimeric polypeptide, which includes mass tag sequences for two or more different target proteins that are separated by one or more protein cleavage agent sites recognized by the protein cleavage agent, and the mass tag sequences of the target proteins, which include one or more intrinsic protein cleavage sites recognized by the protein cleavage agent that recognizes the cleavage sites in the chimeric polypeptide, are separated into their constituent mass tag sequences by the protein cleavage agent. Either the chimeric polypeptide or the target proteins are isotopically-labeled. The combined sample is analyzed mass spectrometry to provide a mass spectrum.

Assuming the chimeric polypeptide is isotopically labeled and the target proteins are not, target proteins in the sample can be identified based on the presence of a mass signal in the mass spectrum that appears at a mass-to-charge ratio that is characteristic of an unlabeled mass tag sequence for the one or more target proteins. Corresponding labeled mass tag sequence for the one or more proteins from the chimeric polypeptide can also be located based on the presence of a mass signal in the mass spectrum that appears at a mass-to-charge ratio characteristic of the labeled mass tag from the chimeric polypeptide. An absolute amount or concentration of the one or more target proteins in the protein sample can then be calculated using the known amount of the chimeric polypeptide added to the protein sample and a ratio of intensities of mass signals for the unlabeled and the corresponding labeled mass tag sequences.

In another embodiment, a set of spectrometric mass tag sequences including chimeric polypeptide cleavage products that are isotopic analogs of a corresponding set of mass tag sequences from pre-selected target proteins is disclosed. In one, non-limiting example, the chimeric polypeptide is designed to produce cleavage products that differ form the corresponding set of mass tag sequences from pre-selected target proteins by a predictable mass difference. In a further, non-limiting example, the target proteins share a common property.

In yet another embodiment, a kit for performing high-throughput quantitative mass spectrometric analysis of a protein sample is disclosed. The kit includes a chimeric polypeptide that includes different mass tag sequences for two or more different target proteins where the target proteins correspond to and are identified by the different mass tag sequences. Each mass tag sequence of the chimeric polypeptide comprises an isotopic analog of a corresponding mass tag sequence of its target protein that is detectable by mass spectrometry as distinct from the mass tag sequence of the target protein. The mass tag sequences of the chimeric polypeptide are separable from each other by a protein cleavage agent at one or more protein cleavage sites in the chimeric polypeptide. The kit also includes instructions for using the chimeric polypeptide to predict quantities of target proteins present in the sample. In some particular embodiments, the chimeric polypeptide is provided in a known concentration. Alternatively or in addition, the kit can include instructions for determining the concentration or amount of chimeric polypeptide. The instruction can also include instructions for using the known amount of chimeric polypeptide as an internal standard for absolute quantitation of the target proteins in the sample by the mass spectrometry, and/or for treating the chimeric polypeptide and the target proteins in the sample with the same protein cleavage agent to provide mass tag sequences of the chimeric polypeptide and the target proteins. Instructions for mixing the chimeric polypeptide and the sample prior to treating with the protein cleavage agent can also be included. The chimeric polypeptide can be isotopically-labeled, or the kit can include instruction for isotopically labeling the target proteins of the sample. Reagents for isotopically labeling the sample can further be included in the kit.

Additional advantages result from expressing multiple different peptide standards from multiple different proteins as a chimeric polypeptide (a hetero-chimeric approach, in which the polypeptide is expressed as a heteropolymer of standards derived from the different proteins). The hetero-chimeric approach avoids complications associated with expression of multiple copies of a single nucleic acid sequence (a homo-chimeric approach). For example, the homo-chimeric approach complicates use of PCR-based gene synthesis because of the redundant complementarity of nucleic acid sequences for multiple copies of a single peptide. The problem with a homo-chimeric approach arises because the PCR-based nucleic acid synthesis approach used in assembly PCR relies on specific hybridization of the oligonucleotides at each assembly step and involves mixing together all of the synthetic oligonucleotide primers at once. Since multiple specific hybridizations are possible in a homo-chimeric approach, the efficiency of the reaction suffers. In addition, the recent approach used to synthetically produce PhiX174 phage (Smith et al. PNAS, 100: 15440-15445, 2003) includes a module of steps that also would be expected less efficient where a homo-chimeric polypeptide is synthesized. Although there are some methods of avoiding the multiple specific hybridizations that exist when a homo-chimera is synthesized, all such methods rely on determining conditions that produce higher specificity and do not address the problem's source, that is, the multiple possible specific hybridizations.

The heterochimeric approach also provides for more efficient and less costly production of an expression vector as measured using a variety of metrics. Since it is more expensive to generate oligonucleotides for PCR synthesis of a nucleic acid sequence coding for a homo-chimeric polypeptide, the hetero-chimeric approach is less costly on a per peptide basis (oligonucleotide cost). Furthermore, the number of PCR or other gene synthesis reactions that are performed and optimized is less by a factor equal to the number of peptides present in a hetero-chimeric polypeptide, so the hetero-chimeric approach is less expensive on a per reaction basis (reaction cost).

Once synthesized, a nucleic acid sequence coding for a chimeric polypeptide is typically confirmed by sequencing. Since the number of sequencing reactions needed to confirm the sequence of a nucleic acid sequence coding for a hetero-chimeric polypeptide is less than for sequencing a nucleic acid sequence coding for a homo-chimeric polypeptide (by a factor equal to the number of peptides present in the heteropolymer), additional savings are provided by the hetero-chimeric approach (sequence cost). Furthermore, the cost of commercial production of expression vectors is on a per base pair basis, so repetition of the peptide sequence in the vector increases the cost of expression of the peptide in the construct (commercial cost).

Once an expression vector for a polypeptide has been produced, the hetero-chimeric approach is significantly more efficient in terms of the lower total number of expression experiments required for a given set of peptides. In the hetero-chimeric approach, each expressed vector provides a set of peptides (such as about 10 peptide standards). Expression, purification and quantitation of the hetero-chimeric polypeptide provide a reduction in the labor and cost needed to make a given set of peptides (expression/purification/quantitation labor and cost) and creates the possibility of smaller “microscale” expression experiments (by about the number of peptides per polypeptide). This is important because the amounts of peptide required are so small in isolated applications. The hetero-chimeric approach makes use of larger format systems, and efficiencies arise because the resources assigned to any given expression vector are split equally among the peptides produced in a single chimeric polypeptide.

A hetero-chimeric approach also is less sensitive to peptide sequences that are difficult to express. For example, in the homo-chimeric approach, difficulties with particular peptide sequences are amplified. However, such difficulties are alleviated by adding such problem peptides to sets of unrelated peptides in a hetero-chimeric polypeptide. The heteropolymer approach also produces defined products for cleavage reactions of the expressed chimeric polypeptide. For instance, if the heteropolymer is represented as ABCDEFG, then each of the specific cleavage sites will give rise to products which are relatively unique as compared to the homopolymer AAAAAAA. This property is useful in confirming complete digestion of controls when added as chimeric polypeptides as well as in reaction tracking and trouble-shooting when performing peptide digestion/purification prior to application of the peptide standards in a mass spectrometric experiment.

There are other unique features of a hetero-chimeric polypeptide standard that are not available to a homo-chimeric polypeptide standard. For example, non-standard peptides can be incorporated, for example, to provide a measure of chemical exposure (such as oxidation, asparagine deamidation and other covalent modifications), or to provide a terminal peptide which can be separately quantitated to provide an indication of the absence of terminal truncations (which would interfere with the assumption that the UV absorbing sequence is reporting the concentration of the fragmented peptide units). In addition, there is freedom to place intervening sequences between peptides that facilitate optimal and facile cleavage of the cleavage site under conditions that would avoid non-specific cleavage reactions.

The hetero-chimeric approach also is more efficient in terms of maintenance of peptide standards. For example, less space is required in low temperature freezers to store host cells (for example, glycerol/buffer solutions of early log phase bacteria) since fewer different host cells are needed for a particular collection of peptide standards.

The following examples are provided to illustrate particular features of certain embodiments. However, the particular features described below should not be construed as limitations on the scope of the invention, but rather as examples from which equivalents will be recognized by those of ordinary skill in the art.

### EXAMPLE 1

### Isotopically-Labeled Peptide Standards for Quantitative Analysis of the Enzymes of the Purine Nucleotide Cycle

The purine nucleotide cycle is a metabolic cycle that is important for replenishing citric acid cycle intermediates and increasing ATP production in exercising muscle. The cycle also plays a central role in general purine metabolism. Three enzymes catalyze the reactions of the purine nucleotide cycle: adenylosuccinate synthetase, AMP deaminase and adenylosuccinate lyase. An imbalance in the enzymatic activities of these and other enzymes involved in purine metabolism has been implicated in the transformation and/or progression of cancer cells in the kidney, liver and colon (see, Weber, “Enzymes of Purine Metabolism in Cancer,” Clin. Biochem., 16: 57-63, 1983). In particular, it appears that the enzymatic activities of the anabolic enzymes such as adenylosuccinate synthetase, adenylosuccinate lyase and AMP deaminase increase in cancer cells. For example, the enzymatic activities of these three enzymes were found to be 3.1, 1.8, and 5.5 times greater, respectively, in liver carcinoma cells than in normal liver.

In this example, isotopically-labeled peptide standards for the purine nucleotide cycle enzymes are expressed as a chimeric polypeptide in E. coli cells grown on an isotopically-altered medium and harvested. The chimeric polypeptide comprises multiple mass tags for the multiple different enzymes of the purine nucleotide cycle expressed as a single amino acid sequence in which the mass tags are separable by cleavage with a protein cleavage agent that recognizes cleavage sites between the mass tags. While a single peptide may be the mass tag for a particular protein, in this instance, the mass tags for each of the enzymes adenylosuccinate synthetase, AMP deaminase and adenylosuccinate lyase are sets of different peptides. The sets of different peptides are present in the chimeric polypeptide and all of the individual peptides that make up the mass tags for the enzymes are separated by endoprotease cleavage sites that are recognized by an endoprotease. Although the cleavage sites may be recognized and cleaved by one or more endoproteases, in this case, the cleavage sites are all recognized and cleaved by they same endoprotease. Upon treatment with the endoprotease, the chimeric polypeptide is cleaved, thereby releasing the peptides that comprise the mass tags of the enzymes. The endoprotease used in this example is trypsin and the cleavage sites are trypsin cleavage sites.

The isotopically-labeled peptide standards produced as an isotopically labeled chimeric polypeptide may be used to quantify the absolute amounts of these enzymes, for example, in normal and cancerous human kidney, liver and colon cells (See Example 2). A comparison of the levels in the normal and cancerous cells reveals whether the increase in enzymatic activity in cancerous cells is due to increased amounts of the enzymes, an increase in the catalytic activity of the enzymes, or some combination thereof. Furthermore, since the absolute amounts of enzymes are determined, absolute catalytic activities for the enzymes may be calculated and compared between normal and cancer cells.

The amino acid sequences of human adenylosuccinate synthetase (Q8N142), human AMP deaminase (P23109) and human adenylosuccinate lyase (P30566) are obtained from the SwissProt database (ExPASY, Geneva, Switzerland). The sequences are then entered into MS-Digest (UCSF Mass Spectrometer Facility, San Francisco, Calif.), which performs an in silico cleavage of a protein sequence with a chosen enzyme, and computes the masses of the generated peptides for a given mass spectrometric technique. The MS-Digest program also can calculate additional parameters for the peptides that may be useful in selecting peptides that are easily separated by chromatography (such as by HPLC) or parameters that may be useful for selecting peptides that are likely to provide strong mass signals when analyzed by a particular technique (for example, the BB parameter; see, Bull and Breese, “Surface Tension of Amino Acid Solutions: A Hydrophobicity Scale of the Amino Acid Residues,” Arch. Biochem. Biophys, 161: 665-670, 1974). An alternate program for in silico digestion of proteins is PeptideMass (ExPASY, Geneva, Switzerland)

Tables 1-3 below show the sequences of the purine nucleotide cycle enzymes adenylosuccinate synthetase, AMP deaminase and adenylosuccinate lyase. The tables also show the cleavage peptides having average masses between about 300 and 2000 amu that are predicted by MS-Digest for treatment of the purine nucleotide cycle enzymes with the endoprotease trypsin (note that monisotopic masses rather than average masses will be detected in by MS analysis). Also shown are the number of methionines that are oxidized in the peptide, the BB parameter (Bull, Henry B. and Breese, Keith, “Surface Tension of Amino Acid Solutions: A Hydrophobicity Scale of the Amino Acid Residues”, Arch. Biochem. Biophys, 161: 665-670), the relative retention of the peptide in a reverse-phase HPLC column (for example, the relative retention of the peptides separated on a C-8 column using a water/acetonitrile gradient) and the sequence positions of the tryptic peptides in the enzyme sequences from which they are derived. The relative elution times of the peptides can also be used as an aid for identifying the peptides, as is well know to those of ordinary skill in the art.

Suitable mass tags for the purine nucleotide cycle enzymes may be selected on the basis of a number of factors. However, in this example, the predicted tryptic peptides having no missed cleavages, a BB (% hydrophobicity) value of greater than about 50% and no methionine residues are selected for each enzyme and used as the mass tags for the enzymes. The peptides selected based on these factors and their masses are shown in Table 4, along with theoretical isoelectric pH values (pI) calculated for the peptides using the Compute pI/Mw tool (ExPASY, Geneva Switzerland). One peptide of 48.8% hydrophobicity, with no missed cleavages or methionine residues (EYDFHLLPSGIINTK) (SEQ ID NO: 6), is selected for adenylosuccinate synthetase so that all of the enzymes have at least 3 peptides in their mass tags.

High hydrophobicities (higher BB parameters) are helpful to the electrospray ionization process when performing ESI-MS because there is a rough correlation between ionization efficiency and hydrophobicity. Moreover, peptides are better separated in an initial separation step on a reverse phase column if they all have sufficient hydrophobicities to be retained by the non-polar columns used in reverse phase separations. Having no missed cleavages within each of the peptides and having no methionine residues in the peptides make the peptides' concentrations more likely to correspond in a one-to-one manner with the concentrations of the proteins from which they are derived. For example, where missed endoprotease cleavage sites are present in a peptide, the measured signal for the peptide will be smaller than expected because some of the peptide present in the sample may be degraded by cleavage at the missed sites. Thus, a calculation of the concentration of a sample protein based on the signal for the peptide will overestimate the sample protein's concentration. Likewise, if methionine residues are oxidized, the amount of methionine-containing peptides will be lower than expected, also leading to overestimation of sample protein concentrations.

Although not considered in the remainder of this example, the pI values of the peptides may be considered to further narrow the number of peptides from each protein. The pI value relates to the relative number of acidic and basic amino acids in a peptide, and may be used to predict whether or not a protein is more likely to become protonated or deprotonated. Acidic proteins (lower pI peptides, such as those having a pI below 7) are more likely to lose protons and take on a negative charge. Basic proteins (higher pI peptides, such as those having a pI above 7) are more likely to gain protons and take on a positive charge. Peptides may, for example, be selected to represent a range of pIs to provide for efficient separation of the peptides using an ion exchange column.

Although not considered in this example, additional factors may be considered in choosing the sets of peptides (mass tags) that are included in one or more chimeric polypeptides. For example, a set of peptides can be selected to exhibit a range of non-overlapping HPLC relative retention factors (a value of between 10 and 60 indicates that a peptide will be retained and separated on a reverse-phase column), a range of non-overlapping masses that correspond with the range of masses detected by the mass spectrometric technique employed (for example, a range of m/z ratios of from about 100 to about 2000). Alternatively, the peptides can be selected according to the presence or absence of particular amino acids (such as rare amino acids like Cysteine or amino acids that may be post-translationally modified or are labile such as asparagine, aspartic acid, proline and glycine). Non-overlapping HPLC retention factors help to provide peptides that may be easily fractionated using HPLC prior to MS analysis. The range of masses detected by a particular mass spectrometric technique is important since peptides with masses outside of the instruments range will not be detected. In addition, particular ranges of masses may be optimal for resolution by a particular mass spectrometric technique, and may be chosen. The presence or absence of particular amino acids may be considered if particular amino acids are likely to be altered (such as methionine which is oxidized). If the peptides are subjected to tandem mass spectrometry to assist in their identification, the presence of rare amino acids makes their identification, and the identification of the proteins from which they are derived, easier. In general, peptides can be selected to provide good mass spectral data, and computer models for predicting fragment ion intensities are contemplated as an additional tool for selecting mass tags.

Appropriate peptides (mass tags) also may be identified by screening protein databases and scientific literature for potentially useful mass tags, for example, the BLAST database (National Institutes of Health, Bethesda, Md.) or the ExPASY databases (Swiss Institute for Bioinformatics, Geneva, Switzerland). Selection of mass tags by mass spectrometry is discussed below in Example 5.

TABLE 1
Adenylate synthetase

Adenylosuccinate synthetase sequence

(SEQ ID NO: 1)

MSGTRASNDRPPGAGGVKRGRLQQEAAATGSRVTVVLGAQWGDEGKGKVV
DLLATDADIISRCQGGNNAGHTVVVDGKEYDFHLLPSGIINTKAVSFIGN
GVVIHLPGLFEEAEKNEKKGLKDWEKRLIISDRAHLVFDFHQAVDGLQEV
QRQAQEGKNIGTTKKGIGPTYSSKAARTGLRICDLLSDFDEFSSRFKNLA
HQHQSMFPTLEIDIEGQLKRLKGFAERIRPMVRDGVYFMYEALHGPPKKI
LVEGANAALLDIDFGTYPFVTSSNCTVGGVCTGLGIPPQNIGDVYGVVKA
YTTRVGIGAFPTEQINEIGGLLQTRGHEWGVTTGRKRRCGWLDLMILRYA
HMVNGFTALALTKLDILDVLGEVKVGVSYKLNGKRIPYFPANQEMLQKVE
VEYETLPGWKADTTGARRWEDLPPQAQNYIRFVENHVGVAVKWVGVGKSR
ESMIQLF

Predicted Tryptic Cleavage Peptides

| | | BB (% | | | | |
|---|---|---|---|---|---|---|
| | | Hydro- | | | | |
| m/z | Modifi- | phobi- | | | | Missed |
| (av) | cations | city) | HPLC | Start | End | Cleavages |
| 551.6464 | | 25.2 | −2.5 | 1 | 5 | 0 |
| 567.6457 | 1 Met-ox | 25.2 | −2.5 | 1 | 5 | 0 |
| 579.6375 | | 28.6 | 14.6 | 223 | 227 | 0 |
| 587.7043 | | 27.9 | 5.8 | 381 | 385 | 1 |
| 611.6799 | | 33.6 | 11.2 | 300 | 304 | 0 |
| 633.7271 | | 28.8 | −2.4 | 159 | 164 | 0 |
| 645.7845 | | 41.0 | 17.2 | 443 | 448 | 0 |
| 652.7734 | | 44.9 | 3.9 | 375 | 380 | 0 |
| 660.7093 | | 9.5 | −5.3 | 153 | 158 | 0 |
| 691.7234 | | 15.7 | 8.5 | 411 | 417 | 0 |
| 716.8612 | | 55.0 | 22.6 | 128 | 133 | 0 |
| 733.8072 | | 28.9 | −1.0 | 123 | 127 | 1 |
| 744.8776 | | 25.8 | 27.0 | 175 | 181 | 1 |
| 761.9023 | | 27.5 | −6.1 | 159 | 165 | 1 |
| 772.0097 | | 47.5 | 13.6 | 228 | 233 | 0 |
| 788.0091 | 1 Met-ox | 47.5 | 13.6 | 228 | 233 | 0 |
| 816.9822 | | 26.4 | −5.1 | 116 | 122 | 2 |
| 820.9732 | | 37.5 | 30.9 | 221 | 227 | 1 |
| 847.9120 | | 15.1 | 4.9 | 411 | 418 | 1 |
| 868.0463 | | 55.5 | 39.9 | 451 | 457 | 0 |
| 873.0498 | | 48.7 | 19.0 | 127 | 133 | 1 |
| 876.0065 | | 37.0 | 17.7 | 120 | 126 | 1 |
| 884.0457 | 1 Met-ox | 55.5 | 39.9 | 451 | 457 | 0 |
| 889.0518 | | 34.7 | 9.5 | 443 | 450 | 1 |
| 910.0212 | | 36.3 | 4.1 | 166 | 174 | 0 |
| 977.1618 | | 34.2 | 27.3 | 220 | 227 | 2 |
| 1004.1817 | | 34.8 | 14.0 | 119 | 126 | 2 |
| 1032.1951 | | 33.7 | 14.1 | 120 | 127 | 2 |
| 1038.1964 | | 34.7 | 0.4 | 165 | 174 | 1 |
| 1065.2658 | | 39.8 | 13.3 | 375 | 384 | 1 |
| 1100.1865 | | 25.8 | 5.0 | 326 | 335 | 0 |
| 1111.3136 | | 46.6 | 32.2 | 449 | 457 | 1 |
| 1127.3130 | 1 Met-ox | 46.6 | 32.2 | 449 | 457 | 1 |
| 1132.2262 | | 20.2 | 26.1 | 22 | 32 | 0 |
| 1199.4042 | | 38.9 | 20.7 | 432 | 442 | 0 |
| 1208.3684 | | 30.4 | 15.1 | 166 | 177 | 1 |
| 1214.4540 | | 54.0 | 55.8 | 364 | 374 | 0 |
| 1220.5515 | | 59.1 | 71.6 | 339 | 348 | 0 |
| 1221.4544 | | 37.2 | 9.7 | 375 | 385 | 2 |
| 1226.3431 | | 20.5 | 4.7 | 6 | 18 | 0 |
| 1228.3617 | | 25.2 | 1.3 | 326 | 336 | 1 |
| 1236.5508 | 1 Met-ox | 59.1 | 71.6 | 339 | 348 | 0 |
| 1275.4131 | | 19.1 | −7.7 | 153 | 164 | 1 |
| 1332.6239 | | 38.9 | 28.2 | 223 | 233 | 1 |
| 1336.5436 | | 29.6 | 11.4 | 165 | 177 | 2 |
| 1345.4671 | | 18.4 | 21.3 | 20 | 32 | 1 |
| 1348.6233 | 1 Met-ox | 38.9 | 28.2 | 223 | 233 | 1 |
| 1376.7401 | | 54.7 | 68.0 | 338 | 348 | 1 |
| 1382.5317 | | 19.8 | 1.1 | 6 | 19 | 1 |
| 1384.5503 | | 24.0 | −2.3 | 326 | 337 | 2 |
| 1392.7395 | 1 Met-ox | 54.7 | 68.0 | 338 | 348 | 1 |
| 1403.5883 | | 19.2 | −11.4 | 153 | 165 | 2 |
| 1431.6451 | | 43.1 | 21.6 | 123 | 133 | 2 |
| 1450.6418 | | 46.1 | 28.9 | 399 | 410 | 0 |
| 1459.6526 | | 34.9 | 37.3 | 33 | 46 | 0 |
| 1501.6557 | | 17.9 | 17.7 | 19 | 32 | 2 |
| 1501.7311 | | 45.9 | 59.2 | 49 | 62 | 0 |
| 1532.9287 | | 51.0 | 64.4 | 337 | 348 | 2 |
| 1548.9281 | 1 Met-ox | 51.0 | 64.4 | 337 | 348 | 2 |
| 1556.7081 | | 20.9 | −15.8 | 63 | 78 | 0 |
| 1573.9596 | | 42.1 | 44.5 | 221 | 233 | 2 |
| 1579.8703 | | 44.8 | 57.7 | 386 | 398 | 0 |
| 1589.9590 | 1 Met-ox | 42.1 | 44.5 | 221 | 233 | 2 |
| 1595.8697 | 1 Met-ox | 44.8 | 57.7 | 386 | 398 | 0 |
| 1595.7726 | | 18.4 | −3.7 | 6 | 21 | 2 |
| 1630.8120 | | 39.4 | 46.4 | 419 | 431 | 0 |
| 1635.8755 | | 31.8 | 31.1 | 166 | 181 | 2 |
| 1637.9538 | | 43.1 | 85.0 | 349 | 363 | 0 |
| 1644.8800 | | 32.1 | 32.4 | 33 | 48 | 1 |
| 1647.8124 | | 45.6 | 44.1 | 182 | 195 | 0 |
| 1652.9002 | | 32.5 | −2.0 | 159 | 174 | 2 |
| 1653.9532 | 1 Met-ox | 43.1 | 85.0 | 349 | 363 | 0 |
| 1686.9585 | | 41.7 | 54.3 | 47 | 62 | 1 |
| 1724.9885 | | 45.4 | 59.4 | 234 | 248 | 0 |
| 1736.0589 | | 42.3 | 54.1 | 385 | 398 | 1 |
| 1738.0749 | | 44.4 | 49.4 | 443 | 457 | 2 |
| 1740.9879 | 1 Met-ox | 45.4 | 59.4 | 234 | 248 | 0 |
| 1748.0014 | | 48.8 | 57.4 | 79 | 93 | 0 |
| 1752.0583 | 1 Met-ox | 42.3 | 54.1 | 385 | 398 | 1 |
| 1754.0742 | 1 Met-ox | 44.4 | 49.4 | 443 | 457 | 2 |
| 1758.9661 | | 21.8 | 2.2 | 1 | 18 | 1 |
| 1774.9655 | 1 Met-ox | 21.8 | 2.2 | 1 | 18 | 1 |
| 1787.0006 | | 37.4 | 42.8 | 418 | 431 | 1 |
| 1826.1655 | | 39.6 | 37.9 | 432 | 448 | 1 |
| 1848.2041 | | 50.8 | 59.7 | 364 | 380 | 1 |
| 1853.1637 | | 43.8 | 55.7 | 234 | 249 | 1 |
| 1869.1631 | 1 Met-ox | 43.8 | 55.7 | 234 | 249 | 1 |
| 1915.1548 | | 21.2 | −1.4 | 1 | 19 | 2 |
| 1923.1654 | | 47.0 | 59.6 | 182 | 197 | 1 |
| 1931.1541 | 1 Met-ox | 21.2 | −1.4 | 1 | 19 | 2 |

TABLE 2
AMP Deaminase

AMP Deaminase Sequence

(SEQ ID NO: 2)

MPLFKLPAEEKQIDDAMRNFAEKVFASEVKDEGGRQEISPFDVDEICPIS
HHEMQAHIFHLETLSTSTEARRKKRFQGRKTVNLSIPLSETSSTKLSHID
EYISSSPTYQTVPDFQRVQITGDYASGVTVEDFEIVCKGLYRALCIREKY
MQKSFQRFPKTPSKYLRNIDGEAWVANESFYPVFTPPVKKGEDPFRTDNL
PENLGYHLKMKDGVVYVYPNEAAVSKDEPKPLPYPNLDTFLDDMNFLLAL
IAQGPVKTYTHRRLKFLSSKFQVHQMLNEMDELKELKNNPHRDFYNCRKV
DTHIHAAACMNQKHLLRFIKKSYQIDADRVVYSTKEKNLTLKELFAKLKM
HPYDLTVDSLDVHAGRQTFQRFDKFNDKYNPVGASELRDLYLKTDNYING
EYFATIIKEVGADLVEAKYQHAEPRLSIYGRSPDEWSKLSSWFVCNRIHC
PNMTWMIQVPRIYDVFRSKNFLPHFGKMLE

Predicted Tryptic Cleavage Peptides

| | | BB (% | | | | |
|---|---|---|---|---|---|---|
| | | Hydro- | | | | |
| m/z | Modifi- | phobi- | | | | Missed |
| (av) | cations | city) | HPLC | Start | End | Cleavages |
| 533.5218 | | 10.8 | −16.0 | 31 | 35 | 0 |
| 575.7559 | | 48.0 | 21.1 | 143 | 147 | 0 |
| 581.6941 | | 51.3 | 27.3 | 266 | 270 | 0 |
| 588.7296 | | 49.7 | 31.4 | 338 | 342 | 0 |
| 607.7323 | | 49.2 | 35.7 | 343 | 347 | 0 |
| 608.6763 | | 29.8 | 10.0 | 19 | 23 | 0 |
| 635.8521 | | 64.0 | 46.2 | 1 | 5 | 0 |
| 635.7487 | | 26.3 | 10.4 | 76 | 80 | 1 |
| 637.6804 | | 14.2 | −12.0 | 288 | 292 | 0 |
| 651.8514 | 1 Met-ox | 64.0 | 46.2 | 1 | 5 | 0 |
| 651.7858 | | 65.0 | 39.3 | 389 | 393 | 0 |
| 663.7622 | | 24.5 | 10.5 | 75 | 79 | 1 |
| 677.7426 | | 33.0 | 1.8 | 258 | 262 | 0 |
| 679.7586 | | 26.3 | 15.8 | 367 | 371 | 0 |
| 686.7881 | | 35.3 | 14.5 | 6 | 11 | 0 |
| 696.8269 | | 48.2 | 5.9 | 330 | 335 | 0 |
| 708.8410 | | 53.6 | 23.6 | 426 | 431 | 0 |
| 720.7648 | | 31.1 | 9.5 | 191 | 196 | 0 |
| 779.9173 | | 42.6 | 18.6 | 24 | 30 | 0 |
| 791.9374 | | 23.7 | 6.8 | 74 | 79 | 2 |
| 791.9374 | | 23.7 | 6.8 | 75 | 80 | 2 |
| 801.9269 | | 30.2 | 8.6 | 718 | 724 | 0 |
| 804.9708 | | 38.3 | 18.7 | 158 | 164 | 1 |
| 812.9500 | | 61.5 | 28.7 | 462 | 467 | 0 |
| 817.9043 | | 39.6 | 3.7 | 293 | 298 | 0 |
| 823.0298 | | 53.7 | 43.6 | 264 | 270 | 1 |
| 826.9964 | | 35.1 | −3.3 | 148 | 153 | 1 |
| 833.0472 | | 39.6 | 10.3 | 143 | 149 | 1 |
| 833.9313 | | 29.3 | −1.8 | 258 | 263 | 1 |
| 842.9958 | 1 Met-ox | 35.1 | −3.3 | 148 | 153 | 1 |
| 846.0210 | | 40.8 | 20.6 | 336 | 342 | 1 |
| 848.8934 | | 31.3 | −0.5 | 432 | 438 | 0 |
| 848.9593 | | 29.5 | 9.8 | 12 | 18 | 0 |
| 848.9400 | | 29.4 | 5.8 | 190 | 196 | 1 |
| 849.0680 | | 52.2 | 52.0 | 343 | 349 | 1 |
| 864.9587 | 1 Met-ox | 29.5 | 9.8 | 12 | 18 | 0 |
| 865.0267 | | 44.6 | 20.4 | 161 | 167 | 1 |
| 900.9757 | | 26.8 | 5.2 | 419 | 425 | 0 |
| 910.0705 | | 40.7 | 31.8 | 154 | 160 | 1 |
| 914.0121 | | 37.1 | 19.5 | 372 | 378 | 1 |
| 927.1884 | | 61.2 | 56.4 | 314 | 320 | 1 |
| 946.0795 | | 36.7 | 0.0 | 293 | 299 | 1 |
| 954.1183 | | 40.8 | −4.9 | 330 | 337 | 1 |
| 958.1155 | | 27.8 | 5.0 | 717 | 724 | 1 |
| 960.1308 | | 46.6 | 50.8 | 470 | 477 | 0 |
| 968.0174 | | 32.1 | 6.0 | 322 | 329 | 0 |
| 979.2184 | | 48.3 | 40.0 | 263 | 270 | 2 |
| 1008.1322 | | 26.0 | −2.8 | 285 | 292 | 1 |
| 1018.1464 | | 29.7 | 3.4 | 518 | 526 | 0 |
| 1028.2039 | | 51.2 | 20.9 | 462 | 469 | 1 |
| 1031.1582 | | 33.3 | 19.6 | 409 | 418 | 0 |
| 1034.1458 | 1 Met-ox | 29.7 | 3.4 | 518 | 526 | 0 |
| 1055.3636 | | 56.0 | 52.7 | 314 | 321 | 2 |
| 1065.3344 | | 49.8 | 42.2 | 139 | 147 | 1 |
| 1070.2007 | | 32.5 | 28.4 | 367 | 374 | 1 |
| 1075.2669 | | 36.9 | 14.5 | 258 | 265 | 2 |
| 1088.2817 | | 37.5 | 18.7 | 150 | 157 | 1 |
| 1096.1926 | | 30.7 | 2.3 | 321 | 329 | 1 |
| 1104.2811 | 1 Met-ox | 37.5 | 18.7 | 150 | 157 | 1 |
| 1106.2314 | | 37.3 | 20.1 | 379 | 388 | 0 |
| 1112.3039 | | 46.9 | 32.3 | 439 | 447 | 0 |
| 1122.3177 | | 52.2 | 70.9 | 632 | 640 | 0 |
| 1175.3847 | | 41.3 | 43.0 | 468 | 477 | 1 |
| 1177.4386 | | 49.4 | 67.1 | 338 | 347 | 1 |
| 1228.4600 | | 34.3 | 1.1 | 674 | 684 | 0 |
| 1237.4971 | | 47.0 | 41.0 | 158 | 167 | 2 |
| 1244.4594 | 1 Met-ox | 34.3 | 1.1 | 674 | 684 | 0 |
| 1294.4158 | | 29.4 | 2.6 | 24 | 35 | 1 |
| 1303.6169 | | 48.4 | 60.7 | 1 | 11 | 1 |
| 1319.6163 | 1 Met-ox | 48.4 | 60.7 | 1 | 11 | 1 |
| 1322.6258 | | 44.1 | 31.4 | 139 | 149 | 2 |
| 1323.5698 | | 35.4 | 23.8 | 718 | 728 | 1 |
| 1323.5475 | | 35.9 | 29.9 | 154 | 164 | 2 |
| 1339.5692 | 1 Met-ox | 35.4 | 23.8 | 718 | 728 | 1 |
| 1343.5322 | | 35.5 | 21.3 | 527 | 537 | 0 |
| 1345.5731 | | 33.7 | 7.9 | 148 | 157 | 2 |
| 1361.5725 | 1 Met-ox | 33.7 | 7.9 | 148 | 157 | 2 |
| 1369.5703 | | 37.2 | 28.6 | 19 | 30 | 1 |
| 1383.7290 | | 40.9 | 17.8 | 143 | 153 | 2 |
| 1399.7284 | 1 Met-ox | 40.9 | 17.8 | 143 | 153 | 2 |
| 1418.7743 | | 51.1 | 83.4 | 338 | 349 | 2 |
| 1434.7300 | | 44.3 | 56.3 | 336 | 347 | 2 |
| 1436.5614 | | 28.0 | −8.3 | 288 | 298 | 1 |
| 1438.6123 | | 29.6 | 19.8 | 12 | 23 | 1 |
| 1444.6166 | | 30.6 | −14.5 | 685 | 697 | 0 |
| 1450.5112 | | 32.0 | −0.9 | 505 | 517 | 0 |
| 1454.6117 | 1 Met-ox | 29.6 | 19.8 | 12 | 23 | 1 |
| 1460.7522 | | 41.6 | 39.3 | 150 | 160 | 2 |
| 1476.7516 | 1 Met-ox | 41.6 | 39.3 | 150 | 160 | 2 |
| 1479.7584 | | 33.4 | 20.2 | 717 | 728 | 2 |
| 1484.7061 | | 40.3 | 24.4 | 318 | 329 | 2 |
| 1495.7578 | 1 Met-ox | 33.4 | 20.2 | 717 | 728 | 2 |
| 1514.6889 | | 41.1 | 43.4 | 197 | 209 | 0 |
| 1516.7242 | | 32.2 | 24.3 | 6 | 18 | 1 |
| 1523.8246 | | 44.2 | 26.5 | 330 | 342 | 2 |
| 1532.7235 | 1 Met-ox | 32.2 | 24.3 | 6 | 18 | 1 |
| 1538.7111 | | 41.6 | 23.1 | 426 | 438 | 1 |
| 1539.7867 | | 26.3 | 12.4 | 300 | 313 | 0 |
| 1555.7861 | 1 Met-ox | 26.3 | 12.4 | 300 | 313 | 0 |
| 1564.7366 | | 27.3 | −12.0 | 288 | 299 | 2 |
| 1574.7474 | | 32.6 | 35.3 | 367 | 378 | 2 |
| 1577.7833 | | 40.2 | 24.7 | 81 | 95 | 0 |
| 1590.7934 | | 39.2 | 28.8 | 419 | 431 | 1 |
| 1610.7780 | | 36.0 | 27.0 | 375 | 388 | 1 |
| 1611.8034 | | 39.8 | 20.8 | 212 | 226 | 0 |
| 1645.8210 | | 39.0 | 11.9 | 322 | 335 | 1 |
| 1667.9619 | | 25.9 | 8.7 | 299 | 313 | 1 |
| 1671.9252 | | 32.2 | −14.7 | 685 | 699 | 1 |
| 1683.9612 | 1 Met-ox | 25.9 | 8.7 | 299 | 313 | 1 |
| 1705.9585 | | 38.9 | 21.0 | 80 | 95 | 1 |
| 1727.1390 | | 44.2 | 34.3 | 448 | 461 | 0 |
| 1738.9939 | | 46.5 | 59.4 | 379 | 393 | 1 |
| 1740.0412 | | 44.2 | 62.5 | 659 | 673 | 0 |
| 1743.1384 | 1 Met-ox | 44.2 | 34.3 | 448 | 461 | 0 |
| 1756.0406 | 1 Met-ox | 44.2 | 62.5 | 659 | 673 | 0 |
| 1759.1378 | 2 Met-ox | 44.2 | 34.3 | 448 | 461 | 0 |
| 1763.0574 | | 40.5 | 44.7 | 271 | 284 | 0 |
| 1762.9695 | | 45.6 | 33.4 | 394 | 408 | 0 |
| 1774.0619 | | 41.1 | 45.3 | 197 | 211 | 1 |
| 1773.9962 | | 37.7 | 8.2 | 321 | 335 | 2 |
| 1779.0568 | 1 Met-ox | 40.5 | 44.7 | 271 | 284 | 0 |
| 1790.0612 | 1 Met-ox | 41.1 | 45.3 | 197 | 211 | 1 |
| 1795.0562 | 2 Met-ox | 40.5 | 44.7 | 271 | 284 | 0 |
| 1807.0132 | | 31.8 | 0.9 | 285 | 298 | 2 |
| 1871.1764 | | 40.0 | 22.7 | 210 | 226 | 1 |
| 1881.0216 | | 37.3 | 45.5 | 700 | 716 | 0 |
| 1884.0688 | | 29.5 | 12.6 | 19 | 35 | 2 |
| 1887.1757 | 1 Met-ox | 40.0 | 22.7 | 210 | 226 | 1 |
| 1903.1124 | | 36.5 | 1.1 | 322 | 337 | 2 |
| 1913.1106 | | 30.6 | 24.8 | 409 | 425 | 1 |
| 1927.1596 | | 39.3 | 49.9 | 350 | 366 | 0 |
| 1942.1740 | | 40.1 | 31.8 | 432 | 447 | 1 |
| 1943.1590 | 1 Met-ox | 39.3 | 49.9 | 350 | 366 | 0 |
| 1946.2436 | | 50.2 | 61.7 | 729 | 744 | 0 |
| 1969.3114 | | 48.9 | 71.7 | 462 | 477 | 2 |
| 2001.2202 | | 37.2 | 39.6 | 372 | 388 | 2 |

TABLE 3
Adenylosuccinate lyase

Adenylosuccinate lyase sequence

(SEQ ID NO: 3)

MAAGGDHGSPDSYRSPLASRYASPEMCFVFSDRYKFRTWRQLWLWLAEAE
QTLGLPITDEQIQEMKSNLENIDFKMAAEEEKRLRHDVMAHVHTFGHCCP
KAAGILHLGATSCYVGDNTDLIILRNALDLLLPKLARVISRLADFAKERA
SLPTLGFTHFQPAQLTTVGKRCCLWIQDLCMDLQNLKRVRDDLRFRGVKG
TTGTQASFLQLFEGDDHKVEQLDKMVTEKAGFKRAFIITGQTYTRKVDIE
VLSVLASLGASVHKICTD1RLLANLKEMEEPFEKQQIGSSAMPYKRNPMR
SERCCSLARHLMTLVMDPLQTASVQWFERTLDDSANRRICLAEAFLTADT
ILNTLQNISEGLVVYPKVIERRIRQELPFMATENIJMAMVKAGGSRQDCH
EKIRVLSQQAASVVKQEGGDNDLIERIQVDAYFSPIHSQLDHLLDPSSFT
GRASQQVQRFLEEEVYPLLKPYESVMKVKAELCL

Predicted Tryptic Cleavage Peptides

| | | BB (% | | | | |
|---|---|---|---|---|---|---|
| | | Hydro- | | | | |
| m/z | Modifi- | phobi- | | | | Missed |
| (av) | cations | city) | HPLC | Start | End | Cleavages |
| 447.4744 | | 11.5 | −2.8 | 392 | 396 | 0 |
| 548.6834 | | 50.9 | 31.0 | 480 | 484 | 0 |
| 578.6965 | | 29.0 | 18.0 | 230 | 234 | 1 |
| 606.7506 | | 39.4 | 14.2 | 195 | 199 | 1 |
| 607.7516 | | 38.2 | −0.9 | 225 | 229 | 0 |
| 623.7510 | 1 Met-ox | 38.2 | −0.9 | 225 | 229 | 0 |
| 630.7265 | | 35.0 | 20.6 | 15 | 20 | 0 |
| 652.8177 | | 32.0 | 1.2 | 304 | 309 | 0 |
| 664.7845 | | 42.6 | 47.2 | 142 | 147 | 0 |
| 671.8637 | | 56.0 | 57.9 | 271 | 276 | 0 |
| 672.8107 | | 39.4 | −4.2 | 368 | 372 | 1 |
| 673.8203 | | 26.0 | −2.2 | 296 | 300 | 1 |
| 689.8197 | 1 Met-ox | 26.0 | −2.2 | 296 | 300 | 1 |
| 720.8714 | | 43.1 | −1.7 | 265 | 270 | 0 |
| 731.8293 | | 36.1 | 9.5 | 219 | 224 | 0 |
| 759.8211 | | 14.1 | −25.3 | 397 | 402 | 0 |
| 765.8989 | | 45.0 | 29.1 | 36 | 40 | 1 |
| 773.8727 | | 35.8 | 10.5 | 189 | 194 | 1 |
| 775.9920 | | 48.5 | 30.8 | 478 | 484 | 1 |
| 807.9035 | | 23.1 | −4.8 | 76 | 82 | 0 |
| 815.0129 | | 44.9 | 26.1 | 135 | 141 | 1 |
| 816.8979 | | 15.9 | 2.2 | 453 | 459 | 0 |
| 821.9172 | | 40.6 | 26.2 | 191 | 196 | 1 |
| 823.9029 | 1 Met-ox | 23.1 | −4.8 | 76 | 82 | 0 |
| 890.0152 | | 24.1 | −13.4 | 297 | 303 | 1 |
| 891.9189 | | 25.2 | 8.9 | 330 | 337 | 0 |
| 906.0146 | 1 Met-ox | 24.1 | −13.4 | 297 | 303 | 1 |
| 930.0614 | | 32.2 | 6.9 | 188 | 194 | 2 |
| 942.1598 | | 42.9 | −1.2 | 368 | 374 | 2 |
| 950.0894 | | 35.5 | 36.5 | 142 | 149 | 1 |
| 964.0921 | | 21.6 | −8.4 | 76 | 83 | 1 |
| 980.0915 | 1 Met-ox | 21.6 | −8.4 | 76 | 83 | 1 |
| 997.2308 | | 54.8 | 80.1 | 126 | 134 | 0 |
| 1011.2362 | | 36.1 | 20.7 | 225 | 233 | 1 |
| 1025.2012 | | 26.8 | −13.6 | 301 | 309 | 1 |
| 1027.2356 | 1 Met-ox | 36.1 | 20.7 | 225 | 233 | 1 |
| 1029.1702 | | 23.5 | −22.3 | 397 | 404 | 1 |
| 1039.1564 | | 36.3 | −2.2 | 277 | 284 | 0 |
| 1046.2038 | | 22.4 | −17.0 | 296 | 303 | 2 |
| 1048.1075 | | 23.6 | 5.3 | 330 | 338 | 1 |
| 1055.1558 | 1 Met-ox | 36.3 | −2.2 | 277 | 284 | 0 |
| 1057.2513 | | 48.0 | 31.3 | 34 | 40 | 2 |
| 1062.2032 | 1 Met-ox | 22.4 | −17.0 | 296 | 303 | 2 |
| 1077.2392 | | 40.0 | 26.1 | 189 | 196 | 2 |
| 1080.1903 | | 40.6 | 16.6 | 67 | 75 | 0 |
| 1106.2780 | | 37.2 | 24.8 | 191 | 199 | 2 |
| 1120.3457 | | 44.5 | 49.6 | 138 | 147 | 1 |
| 1130.3382 | | 35.1 | 32.6 | 405 | 415 | 0 |
| 1167.4248 | | 33.6 | 17.1 | 225 | 234 | 2 |
| 1183.4242 | 1 Met-ox | 33.6 | 17.1 | 225 | 234 | 2 |
| 1188.2722 | | 12.9 | −28.1 | 392 | 402 | 1 |
| 1210.4034 | | 33.7 | 16.8 | 285 | 295 | 0 |
| 1226.4028 | 1 Met-ox | 33.7 | 16.8 | 285 | 295 | 0 |
| 1233.4412 | | 28.3 | 8.0 | 76 | 85 | 2 |
| 1246.2841 | | 25.5 | −5.4 | 416 | 426 | 0 |
| 1249.4406 | 1 Met-ox | 28.3 | 8.0 | 76 | 85 | 2 |
| 1271.4682 | | 43.6 | 42.9 | 235 | 245 | 0 |
| 1320.5576 | | 37.0 | 8.6 | 219 | 229 | 1 |
| 1336.5570 | 1 Met-ox | 37.0 | 8.6 | 219 | 229 | 1 |
| 1337.6592 | | 51.5 | 103.8 | 126 | 137 | 1 |
| 1366.5920 | | 31.8 | 13.2 | 285 | 296 | 1 |
| 1373.7118 | | 49.6 | 56.2 | 265 | 276 | 1 |
| 1382.5914 | 1 Met-ox | 31.8 | 13.2 | 285 | 296 | 1 |
| 1399.6434 | | 41.6 | 39.2 | 235 | 246 | 1 |
| 1399.6873 | | 37.6 | 35.6 | 403 | 415 | 1 |
| 1405.6506 | | 39.5 | 38.9 | 138 | 149 | 2 |
| 1421.4974 | | 23.9 | 7.9 | 1 | 14 | 0 |
| 1427.6568 | | 40.9 | 39.3 | 234 | 245 | 1 |
| 1437.4968 | 1 Met-ox | 23.9 | 7.9 | 1 | 14 | 0 |
| 1457.6213 | | 18.9 | −25.1 | 392 | 404 | 2 |
| 1460.7741 | | 43.8 | 73.3 | 135 | 147 | 2 |
| 1523.8096 | | 27.7 | −12.2 | 297 | 309 | 2 |
| 1539.8090 | 1 Met-ox | 27.7 | −12.2 | 297 | 309 | 2 |
| 1552.7792 | | 44.2 | 34.8 | 21 | 33 | 0 |
| 1555.8320 | | 39.2 | 35.6 | 234 | 246 | 2 |
| 1568.7786 | 1 Met-ox | 44.2 | 34.8 | 21 | 33 | 0 |
| 1691.9968 | | 44.8 | 55.7 | 271 | 284 | 1 |
| 1707.9962 | 1 Met-ox | 44.8 | 55.7 | 271 | 284 | 1 |
| 1724.0422 | | 36.1 | 30.2 | 219 | 233 | 2 |
| 1740.0415 | 1 Met-ox | 36.1 | 30.2 | 219 | 233 | 2 |
| 1793.2204 | | 50.5 | 106.2 | 126 | 141 | 2 |
| 1820.1403 | | 31.3 | 10.4 | 86 | 101 | 0 |
| 1831.1414 | | 39.1 | 60.9 | 230 | 245 | 2 |
| 1836.1397 | 1 Met-ox | 31.3 | 10.4 | 86 | 101 | 0 |
| 1838.1683 | | 45.2 | 65.9 | 247 | 264 | 0 |
| 1844.1316 | | 45.7 | 37.0 | 21 | 35 | 1 |
| 1860.1310 | 1 Met-ox | 45.7 | 37.0 | 21 | 35 | 1 |
| 1865.2004 | | 31.3 | 14.6 | 285 | 300 | 2 |
| 1869.0704 | | 32.9 | 11.8 | 67 | 82 | 1 |
| 1881.1998 | 1 Met-ox | 31.3 | 14.6 | 285 | 300 | 2 |
| 1885.0698 | 1 Met-ox | 32.9 | 11.8 | 67 | 82 | 1 |
| 1897.1991 | 2 Met-ox | 31.3 | 14.6 | 285 | 300 | 2 |
| 1940.3903 | | 47.3 | 65.1 | 172 | 187 | 0 |
| 1956.3897 | 1 Met-ox | 47.3 | 65.1 | 172 | 187 | 0 |
| 1966.3434 | | 43.8 | 62.2 | 246 | 264 | 1 |
| 1967.4348 | | 46.3 | 69.3 | 375 | 391 | 0 |
| 1983.4342 | 1 Met-ox | 46.3 | 69.3 | 375 | 391 | 0 |
| 1999.4335 | 2 Met-ox | 46.3 | 69.3 | 375 | 391 | 0 |

TABLE 4

| | | | | | |
|---|---|---|---|---|---|
| | | | | | SEQ |
| | | | | | ID |
| Peptide Sequence | m/z (avg) | pI | Enzyme | Peptide # | NO: |

| | | | | | |
|---|---|---|---|---|---|
| LIISDR | 716.8612 | 5.84 | Adenylosuccinate | 1 | 4 |
| | | | synthetase | | |
| LDILDVLGEVK | 1214.4540 | 4.03 | Adenylosuccinate | 2 | 5 |
| | | | synthetase | | |
| EYDFHLLPSGIINTK | 1748.0014 | 5.32 | Adenylosuccinate | 3 | 6 |
| | | | synthetase | | |
| FLSSK | 581.6941 | 8.75 | AMP deaminase | 4 | 7 |
| DLYLK | 651.7858 | 5.83 | AMP deaminase | 5 | 8 |
| LSIYGR | 708.8410 | 8.75 | AMP deaminase | 6 | 9 |
| IYDVFR | 812.9500 | 5.84 | AMP deaminase | 7 | 10 |
| NPFLDFLQK | 1122.3177 | 5.84 | AMP deaminase | 8 | 11 |
| YETWCYELNLIAEGLK | 1946.2436 | 4.25 | AMP deaminase | 9 | 12 |
| NALDLLLPK | 997.2308 | 5.84 | Adenylosuccinate | 10 | 13 |
| | | | lyase | | |
| LLANLK | 671.8637 | 8.75 | Adenylosuccinate | 11 | 14 |
| | | | lyase | | |
| AELCL | 548.6834 | 4.00 | Adenylosuccinate | 12 | 15 |
| | | | lyase | | |

The selected peptide sequences are then combined in the order shown from top to bottom (1-12) in Table 4 to yield a single chimeric polypeptide having the following sequence, where the trypsin cleavage sites are indicated as vertical bars:

(SEQ ID NO: 16)

LIISDR|LDILDVLGEVK|EYDFHLLPSGIINTK|FLSSK|DLYLK|L
SIYGR|IYDVFR|NPFLDFLQK|YETWCYELNLIAEGLK|LLANLK|N
ALDLLLPK|AELCL

This chimeric sequence, when treated with trypsin, will be cleaved on the carboxy side of the arginine residues (R) and lysine residues (K) to regenerate the peptides shown in Table 4. In other words, a chimeric sequence that includes tryptic peptides joined directly end-to-end will generally be cleaved by trypsin to regenerate the tryptic peptides it includes. Thus, the sequence specificity used to generate peptides from the proteins in silico is also taken advantage of during in vitro digestion (with the actual cleavage agent) of the chimeric polypeptide to generate the desired peptides from a chimeric polypeptide. Actual digestion of the chimeric polypeptide can take place in the presence or absence of the sample proteins that are to be quantified.

Although not considered further in the remainder of this example, it is possible to construct multiple chimeric polypeptides, each of which includes a subset of all of the desired standard peptides for the multiple different proteins. For example, two chimeric polypeptides, one including peptides 1-6 of Table 4 and one including peptides 7-12 of Table 4 could be designed. Combinations of multiple chimeric polypeptides that each include standards for multiple subsets of proteins of interest may be desirable where the different proteins of interest (or the peptides derived therefrom) differ in some parameter that makes it difficult to provide standard peptides for all of the proteins of interest in a single chimeric polypeptide. Difficulties in trying to combine standard peptides for multiple different proteins in a single chimeric polypeptide may arise where, for example, the proteins of interest are present in a wide range of concentrations in the sample or there are a great number of different proteins of interest (such as more than 50, 100 or 500 such different proteins of interest). In one embodiment, multiple different chimera, each of which contains standard peptides (mass tags) for proteins of interest in a particular concentration range (such as a range of one order of magnitude in concentration) are employed. The different chimera are added to the sample in different concentrations so that the standard peptides are present at a concentration that is substantially similar (such as within an order of magnitude in concentration) to the peptides derived from the sample proteins. Thus, where a set of proteins of varying concentrations are of interest, multiple chimera including different combinations of peptides that are expected to be released from the sample proteins in different concentration ranges may be used.

Additional sequences that facilitate expression may be added to the N-terminus of the sequence [such as met-lys (MK) or met-arg (MR)], provided they are cleaved from the chimeric polypeptide upon treatment with trypsin. Also, sequences that assist purification (such as a poly-histidine affinity tag) or detection (such as a poly-tryptophan tag) may be added, for example, to the C-terminus of the sequence.

Alternative orders of the selected peptides are of course possible in the chimeric polypeptide since each peptide ends with an amino acid after which trypsin will cleave the peptide from the chimeric polypeptide. Intervening spacer amino acid sequences may also be added between the selected peptides in the chimeric polypeptide, provided trypsin treatment removes the spacers and generates the peptides that were selected for expression as the chimeric polypeptide. For example, alternative orders of the selected peptides include 12, 11, 10, 9, 8, 7, 6, 5, 4, 3, 2, 1 and 1, 2, 3, 7, 8, 9, 4, 5, 6, 10, 11, 12 and 7, 9, 5, 3, 2, 1, 5, 8, 12, 6, 12, 4, 11, 10. Regardless of the order, if the tryptic peptides are joined end-to-end the resulting chimeric polypeptide can be digested with trypsin to release the constituent peptides.

The amino acid sequence particularly displayed above is then input into the Backtranslation tool V2.0 (available indirectly through ExPASY, Geneva, Switzerland or directly from Entelechon GmbH, Regensburg, Germany) and converted into the following nucleic acid sequence (using an E. coli-optimized codon usage):

(SEQ ID NO: 17)

TTG ATC ATT TCT GAT AGA CTC GAT ATA CTA GAC GTT
TTA GGT GAA GTA AAA GAG TAC GAC TTT CAT TTA CTA
CCA AGT GGA ATT ATC AAT ACT AAA TTT CTG AGC TCA
AAA GAC CTC TAC CTG AAG TTG TCG ATA TAT GGC AGG
ATT TAC GAC GTG TTT CGA AAC CCG TTC CTA GAT TTC
TTA CAA AAA TAT GAG ACA TGG TGC TAT GAA TTG AAT
CTT ATA GCG GAG GGG CTC AAG CTT CTC GCC AAC TTA
AAG AAT GCA CTA GAT CTT TTG CTT CCT AAG GCT GAA
CTG TGT CTG

If a methionine (M) and a lysine (K) are to be added to the N-terminus to facilitate expression, the following E. Coli-optimized nucleic acid sequence is obtained:

(SEQ ID NO: 18)

ATG AAG TTG ATC ATA TCA GAT AGG CTA GAC ATT CTA
GAT GTA CTA GGT GAG GTG AAG GAG TAT GAC TTT CAT
TTA CTT CCG AGT GGG ATT ATC AAT ACT AAA TTC CTG
TCG TCT AAA GAC CTC TAT TTG AAA CTG AGC ATT TAC
GGA AGA ATA TAC GAT GTT TTT CGA AAC CCT TTC CTT
GAT TTT CTC CAA AAG TAC GAA ACA TGG TGC TAT GAA
TTG AAT TTA ATA GCT GAG GGC CTC AAG CTC CTT GCA
AAC CTA AAA AAT GCG TTA GAC CTG CTT CTG CCA AAA
GCC GAA TTA TGT TTG

In this example, the synthetic nucleic acid sequence shown above is to be cloned into a pENTR/D-TOPO® entry vector (Invitrogen, San Diego, Calif.). Therefore, a CACC sequence is added to the 5′ end to yield the nucleic acid sequence below. The CACC sequence is added to provide directional cloning of the sequence in this system.

(SEQ ID NO: 19)

CACC ATG AAG TTG ATC ATA TCA GAT AGG CTA GAC ATT
CTA GAT GTA CTA GGT GAG GTG AAG GAG TAT GAC TTT
CAT TTA CTT CCG AGT GGG ATT ATC AAT ACT AAA TTC
CTG TCG TCT AAA GAC CTC TAT TTG AAA CTG AGC ATT
TAC GGA AGA ATA TAC GAT GTT TTT CGA AAC CCT TTC
CTT GAT TTT CTC CAA AAG TAC GAA ACA TGG TGC TAT
GAA TTG AAT TTA ATA GCT GAG GGC CTC AAG CTC CTT
GCA AAC CTA AAA AAT GCG TTA GAC CTG CTT CTG CCA
AAA GCC GAA TTA TGT TTG

The nucleic acid sequence immediately above is then synthesized, for example, using assembly PCR (see, for example, Stemmer et al., Gene 164:49-53, 1995). Assembly PCR involves synthesizing overlapping oligonucleotides that cover the desired nucleotide sequence, and using these oligonucleotides as primers for the PCR reaction. The primers are repetitively extended by PCR to assemble the fall length synthetic nucleic acid sequence. The process of oligonucleotide design for synthetic nucleic acid sequence construction by assembly PCR may, for example, be automated by using a computer program. One example of such a program is DNAWorks (Hoover and Lubkowski, Nucleic Acids Res. 30:e43, 2002). The amino acid sequence of a chimeric polypeptide, including the desired flanking sequences necessary for directional cloning, is put into the program, which then reverse-translates the polypeptide sequence into a set of oligonucleotide sequences encoding the chimeric polypeptide. The program optimizes the oligonucleotides to match the codon bias of the host chosen for expression (for example, E. coli) and to have highly homogenous melting temperatures for all of the overlapping oligonucleotide sections.

Any alternative method for synthesizing the nucleic acid sequence may be used. For example, the FokI method of nucleic acid sequence synthesis (Mandecki and Bolling, Gene 68:101-07, 1988) or the self-priming polymerase chain reaction (Dillon and Rosen, Biotechniques 9:298-300, 1990) may be used (see also, Example 8).

Once the synthetic nucleic acid sequence has been constructed, it can be cloned using a variety of well known methods. In one embodiment, traditional restriction digest cloning is used to clone the synthetic nucleic acid sequence (see, for example, Sambrook et al., Molecular Cloning: A Laboratory Manual, published by Cold Spring Harbor Laboratory, 2001) In another embodiment, ligation-independent cloning vectors are used to clone the synthetic nucleic acid sequence (Aslanidis and de Jong, Nucl. Acids Res. 18:6069-74, 1990; Haun et al., Biotechniques 13:515-18, 1992). Ligation-independent cloning vectors are designed for rapid cloning and expression of nucleic acid sequences in multiple expression systems (for example, E. coli, insect cell, and mammalian cell). Ligation-independent cloning was developed for the directional cloning of PCR products without the need for restriction enzyme digestion or ligation reactions. An example of ligation-independent cloning system for high-level inducible expression in E. coli is the pENTR/D-TOPO® entry vector (Invitrogen, San Diego, Calif.). Another example is the pETI00/D-TOPO® system from Invitrogen, which also requires that the forward PCR primer contains the sequence CACC at the 5′ end of the primer. These four nucleotides base pair with a complementary overhang sequence vector. This vector also allows the optional inclusion of an N-terminal histidine tag to facilitate protein purification.

In this example, the synthetic nucleic acid sequence is then amplified, for example, using PCR with a proofreading enzyme combination (such as Platinum® Taq DNA Polymerase, Invitrogen, San Diego, Calif.). The PCR product is then mixed with a Directional TOPO® pENTR™ vector (Invitrogen, San Diego, Calif.), which is used to transform E. coli host cells. Successfully transformed bacteria can be selected according to antibiotic resistance. The sequence of the vector expressed by the host cell can be confirmed, for example, by LC-MS/MS.

The transformed E. coli cells (or other type of host cell) are then grown on an isotopically-altered medium and induced to express the chimeric polypeptide, which desirably is deposited in the cells to form inclusion bodies. As the host cells grow, they incorporate atoms of the medium into the molecules they synthesize, including the expressed chimeric polypeptide. The medium may include substantially only a particular isotope of a particular element, or may include isotopically-labeled precursor amino acids that are specifically incorporated into the expressed chimeric polypeptide. For example, media that include a ¹³C-labeled molecule as the sole carbon source and/or a ¹⁵N-labeled molecule as the sole nitrogen source may be used to provide uniform labeling of the chimeric polypeptide with ¹³C and/or ¹⁵N. Labeled amino acids, such as ²H, ¹³C, ¹⁵N and/or ¹⁸O labeled amino acids, may be included in the medium to provide residue-specific labeling of the chimeric polypeptide.

A number of methods of isotope labeling of the chimera exist. In one specific embodiment, ¹⁵N uniformly labeled medium (Cambridge Isotope Laboratories, Andover, Mass.) is used to label all nitrogen atoms of the chimeric polypeptide (Oda et al., PNAS 96:6591-96, 1999). Such uniformly labeled media include media that include an isotopically-altered molecule as the sole source of a particular element. For example, if ¹⁵NH₄Cl is used as the sole nitrogen source, all of the nitrogen atoms in the chimeric polypeptide will be labeled. Alternatively, a ¹³C-labeled molecule can be used as the sole carbon source. Alternatively, specific, isotopically altered amino acids such as ¹⁵N-containing amino acids, ¹³C-containing amino acids, or deuterium-enriched amino acid precursors (for example, L-leucine-5,5,5-d₃, L-serine-2,3,3-d₃ an/or L-tyrosine-3,3-d₂, Cambridge Isotope Laboratories, Andover, Mass.) are added to the growth medium in place of their unlabeled counterparts and are incorporated into the chimeric polypeptide in a residue-specific manner during protein synthesis (Zhu et al., RCM 16:2115-23, 2002; Ong et al., Mol. Cell. Proteomics 1:376-86, 2002).

In another example, isotopic labeling of the chimeric polypeptide is carried out in bacterial strains that are auxotrophic for one or more amino acids. Biosynthetic enrichment of selected amino acids in a polypeptide with ¹⁵N requires efficient and consistent incorporation of ¹⁵N-containing amino acid precursors into the polypeptide. However, isotopic dilution by endogenous amino acid biosynthesis and “scrambling” of the ¹⁵N label to other types of residues, either through metabolic conversion of one amino acid to another, or as a result of transaminase activity, can interfere with efficient and consistent incorporation of ¹⁵N precursors into the polypeptide. As amino acid biosynthesis is regulated by feedback inhibition, both endogenous amino acid biosynthesis and scrambling can me mitigated by supplementing the growth medium with high concentrations of all 20 amino acids. A more satisfactory approach to controlling endogenous amino acid biosynthesis and scrambling, however, is to use hosts for chimeric polypeptide expression that have been modified to contain the appropriate genetic lesions to control amino acid biosynthesis (see, for example, Muchmore et al., Methods Enzymology, 177:44-73, 1989 and Waugh, J. Bio. NMR, 8:184-92, 1996).

Regardless of the method employed to isotopically label the chimeric polypeptide, it is then isolated from the inclusion bodies. For example, following chimeric polypeptide expression, the bacteria is pelleted in a centrifuge, and if not used immediately the pellets may be stored, for example, at −80° C. Bacterial pellets are either used directly or are thawed (if stored) and then homogenized in a buffer. In one embodiment, homogenization is carried out through extrusion at high pressure (for example, using a French Press). This serves to break open the bacteria and otherwise make the sample ready to be processed. Additional optional treatments include DNase treatment.

The expressed chimeric polypeptide, which as mentioned above is desirably in the form of dense inclusion bodies, remains in the pellet after the pellet is homogenized. The inclusion bodies are then separated from the remaining homogenized bacterial preparation. For example, separation of inclusion bodies from the cell homogenate may be achieved using low-speed centrifugation.

Alternatively, high-speed centrifugation through dense solutions of sucrose may be used. Appropriate methods for isolating inclusion bodies from bacteria are provided, for example, in Georgiou and Valax, Methods in Enzymology, 309: 48-58, 1999.

The chimeric polypeptide is then purified from the inclusion bodies. For example, the inclusion bodies may be dissolved in a protein denaturant, for example, urea, guanidine hydrochloride (GdnHCl), or guanidinium isocyanate. The specific denaturant can be selected to be compatible with one or more subsequent purification steps. The chimeric polypeptide may then be purified from the dissolved inclusion bodies by any of the means known in the art (see, for example, Guide to Protein Purification, ed. Deutscher, Meth. Enzymol. 185, Academic Press, San Diego, 1990 and Scopes, Protein Purification Principles and Practice, Springer Verlag, New York, 1982). In one example, the chimeric polypeptide is expressed with an optional N-terminal histidine tag and is purified from the dissolved inclusion bodies using affinity chromatography. For example, immobilized-metal affinity chromatography (IMAC) (Qiagen, Valencia, Calif.) is used, which takes advantage of the high affinity and specificity of immobilized metal ions (for example, nickel) for the histidine residues of the tag. Additional examples of appropriate purification methods are provided, for example, in Hearn and Acosta, Journal of Molecular Recognition, 14: 323-329, 2001.

In certain embodiments, it is useful to determine the concentration for the purified chimeric polypeptide because the concentration of the chimeric polypeptide may be used to calculate absolute concentrations of sample proteins when the chimeric polypeptide is added to a protein sample in a known amount. The concentration of the purified chimeric polypeptide may, for example, be determined using UV-Vis absorption or HPLC. For example, assuming the chimeric polypeptide including the N-terminal sequence of MK does not have a poly-His affinity purification tag, or such a tag has been removed, the chimeric polypeptide has an extinction coefficient (molar absorptivity) of 13490 M⁻¹ cm⁻¹ at 280 nm in a solution of 6.0 M guanidinium hydrochloride and 0.02 M phosphate buffer at pH 6.5 (calculated using the ProtParam tool, ExPASY, Geneva, Switzerland). A solution containing the purified chimeric polypeptide is placed in a quartz absorption cell of known path-length and the absorbance at 280 nm is measured using a UV-Vis spectrophotometer (for example, a Cary 4000 UV-Vis spectrophotometer, Varian, Palo Alto, Calif.). The concentration of the chimeric polypeptide is determined using Beer's Law (c=A/εl), where “c” is the concentration to be calculated, “A” is the measured absorbance at 280 nm, “ε” is the extinction coefficient (in this instance at 280 nm), and “l” is the path length of the absorption cell. For example, if a solution (6.0 M guanidinium hydrochloride, 0.02 M phosphate buffer, pH 6.5) containing the purified chimeric polypeptide is measured to have an absorbance of 0.100 at 280 nm using an absorption cell having a path length of 1.00 cm, the concentration (c) of the chimeric polypeptide equals 0.100/(13490 M⁻¹ cm⁻¹)(1.00 cm), or 7.41×10⁻⁶M (7.41 μM). This concentration may then be used to calculate a volume of the solution that may be added to a sample to deliver a precise amount of the chimeric polypeptide. For example, to deliver 1 ng (85 pmol) of the unlabeled chimeric polypeptide (MW=11769.9) to a sample, the volume of solution used would be equal to 8.5×10⁻¹¹ mol/7.41×10⁻⁶M, or 1.14×10⁻⁵ L (11.4 μL). In addition to the ProtParam tool discussed above, the extinction coefficient (molar absorptivity) of the chimeric polypeptide may be calculated using the formula: ε²⁸⁰ (M⁻¹ cm⁻¹)=5,690(#Trp)+1,280(#Tyr)+120(#Cys) (see, for example, Gill and von Hippel, Analytical Biochem. 182:319-326, 1989 and Pace et al., Protein Science 4:2411-2423, 1995). Regardless of how the extinction coefficient is calculated, it is used along with the absorbance measured for a chimeric polypeptide sample in an absorption cell of known path length to calculate the concentration of the chimeric polypeptide.

Since the physico-chemical properties of the isotopically-labeled chimeric polypeptide are virtually identical to the unlabeled chimeric polypeptide, the extinction coefficient for the labeled chimeric polypeptide is expected to be the same as the unlabeled chimeric polypeptide. This virtual identity makes it possible to calculate an extinction coefficient for a non-labeled chimeric polypeptide and use it for calculating the concentration of the labeled chimeric polypeptide. Although absorbance measurements can provide a concentration, it also is possible to measure concentrations of peptides by NMR (see, for example, Cavaluzzi et al, Anal Biochem, 308: 373-380, 2002).

If the number and type of heavy isotopes that are incorporated in a labeled version of a peptide are known, it is possible to predict the mass-to-charge ratio of its signal in a mass spectrum. The number of heavy isotopes incorporated in a particular peptide of a chimeric polypeptide may be estimated from the sequence and/or molecular formula of the peptide and the manner by which heavy isotopes are incorporated into the chimeric polypeptide (that is at a predictable number of sites). Where isotopically-labeled amino acids are used to label the chimeric polypeptide's constituent peptides, the labeled peptides will generally have a number of heavy isotopes that is equal to the number of the particular type of amino acid in the sequence of a particular peptide multiplied by the number of heavy isotopes in the labeled amino acid. The mass of the labeled peptide will be equal to the mass of the unlabeled peptide plus the number of heavy isotopes of each type that are incorporated into the labeled peptide multiplied by the difference in mass between the light and heavy versions of the isotope(s) incorporated into the peptide. For example, if a particular peptide contains 5 nitrogen atoms and all of these nitrogen atoms are replaced by ¹⁵N instead of naturally occurring ¹⁴N, the labeled peptide will have a mass that is approximately 5 atomic mass units (amu) greater than the unlabeled peptide. If the mass signal for the unlabeled peptide is identified in a mass spectrum, the mass signal for the labeled peptide may be easily identified by its location at a mass-to-charge ratio that is approximately 5 amu greater than the unlabeled peptide. In general, it is desirable to select a labeling scheme that will provide consistent and predictable labeling of the peptides in the chimeric polypeptide so that the mass signals for the standard peptides released from the chimeric polypeptide may be reliably located. If the labeling scheme does not provide consistent labeling, the mass signal for the labeled peptide will be smaller than expected, and will lead to an overestimation of the concentration of sample proteins. The fidelity of the labeling procedure can be tested by analyzing the peptides with LC/MS.

### EXAMPLE 2

### Absolute Quantitative MS-Analysis of the Enzymes of the Purine Nucleotide Cycle in Normal and Cancerous Kidney Cells

In this example, the chimeric polypeptide of Example 1 is used to determine whether a change in the absolute concentration of the enzymes of the purine nucleotide cycle is evident between normal and cancerous kidney cells. A needle biopsy is performed on a subject to obtain two kidney tissue samples. One of the samples is obtained from normal kidney tissue and the other is obtained from cancerous kidney tissue (for example, a tumor that is identified and located for biopsy by, for example, an imaging technique such as ultrasound, magnetic resonance imaging or computed tomography). The samples are subsequently treated and analyzed separately according to the following procedure.

The normal and cancerous kidney tissue samples are each cut into thin sections, frozen in liquid nitrogen, and ground in a mortar and pestle. A buffer, such as a RIPA buffer (150 mM sodium chloride, 50 mM Tris HCl, pH 7.4, 1 mM EDTA, 1% Triton X-100, 1% sodium deoxycholic acid, 0.1% SDS, 0.2 mM AEBSF, 5 μg/mL) is added and the resulting solution is kept cold. The samples are homogenized using a stator/rotor homogenizer (such as a Brinkman PT-2100) or a French Press, and the protein fraction of each is isolated. Small aliquots of the homogenate may centrifuged, and the supernatant can be stored at low temperature until needed.

In this example, the chimeric polypeptide is expressed in E. coli that are auxotrophic for both lysine and arginine (for example, an E. coli strain that is defective in the argH and lysA genes; see Waugh, “Genetic Tools for Selective Labeling of Proteins with α-¹⁵N-amino acids, Journal of Biomolecular NMR, 8, 184-192, 1996). The bacterial cells are then grown on a medium containing L-Lysine-¹³C₆, ¹⁵N₂ hydrochloride and L-Arginine-¹³C₆, ¹⁵N₄ hydrochloride (Sigma-Aldrich, St. Louis, Mo.). Since all of the tryptic peptides have been selected to contain only one or the other of arginine and lysine (assuming no missed cleavages in the selected peptides of the chimeric polypeptide), all of the tryptic peptides will be reproducibly labeled with a single “heavy” version of lysine or arginine, making it easy to predict the mass of the labeled version of the peptides (and their mass-to-charge ratios in a mass spectrum; see Example 1 above).

Referring again to Table 4 of Example 1, it is seen that tryptic peptides 1, 6 and 7 will have a single “heavy” arginine residue and the remaining peptides will have a single “heavy” lysine residue. In a mass-spectrum, the peptides that are labeled with the “heavy” arginine will be mass-shifted approximately 10 amu (each arginine has 6 heavy 13C atoms that are 1 amu heavier than the naturally-occurring ¹²C atom, and 4 heavy ¹⁵N atoms that are 1 amu heavier than the naturally-occurring ¹⁴N atom) from the unlabeled peptides derived from the sample proteins, and the peptides that are labeled with the “heavy” lysine will be mass-shifted by approximately 8 amu (6 heavy carbons and 2 heavy nitrogens). If additional heavy isotopes are included in the chimeric polypeptide (such as by oxygen exchange with H₂¹⁸O), a determination of the number of such isotopes that are actually incorporated is made to determine the difference in mass between the labeled and unlabeled versions of the different peptides of the chimeric polypeptide. For example, each incorporated ¹⁸O atom will produce a mass-shift of 2 amu relative to the naturally-occurring ¹⁶O.

The labeled chimeric polypeptide is isolated and purified, and the concentration of a solution of the purified chimeric polypeptide is determined (see, Example 1). A volume of the solution that provides approximately 100 pmol of this chimeric polypeptide is added to each of the isolated protein fractions from the normal and cancerous kidney samples. If the sample is too concentrated, a partial injection can be used during subsequent mass spectrometric analysis.

The samples with added chimeric polypeptide are then digested using trypsin (because the chimeric polypeptide was designed using tryptic peptides of the purine nucleotide cycle enzymes). Alternatively, the chimeric polypeptide and the sample are digested separately and then combined for analysis. An exemplary procedure for tryptic digestion is as follow. Total protein is estimated, for example, using the Bradford method. If necessary, the sample buffer is exchanged for a 50 mM ammonium bicarbonate, pH8 buffer. Porcine trypsin (Promega, Madison, Wis.) is added a weight ratio of 100 parts sample protein to 1 part trypsin. Rapigest (0.1-1%, Waters, Beverley Mass.) can be added to aid digestion. The sample is incubated overnight at 37° C. After digestion, the sample is acidified by adding acetic acid to 5%, or until the pH is approximately 3. The sample can then be rapidly frozen and stored until used.

The digested samples are then subjected to mass spectral analysis. Target volumes for analysis depend on the particular sampling scheme, but in the case of autosampling with a Water Autosampler (model 920, Spark Holland), a 100 μL sample can be used. Since several loop loading cycles can be performed for each sample, it is possible to load a larger volume of sample than a single injection allows. The sample is injected into a Symmetry C-18 Opti-pak precolumn (Waters, Milford, Mass.) at a flow rate of 10 μL/min. The operating buffer for this step is 0.2% formic acid in water

In this example, the digested samples are analyzed using an ESI-MS technique (see, Wolters et al., Anal. Chem., 73: 563, 2001 and references therein). Briefly, the peptides are first separated based on charge using strong cation exchange on an appropriate column (such as Polysufloethyl-A, POLY LC Inc.) using gradient elution with an initial buffer (buffer A) of 25% acetonitrile, 0.02% HFBA in water, and a second buffer (buffer B) that further includes 300 mM ammonium acetate. After column equilibration, the sample is loaded and the column is washed with buffer A. Next a series of step gradients using 5%, 10%, 20%, 30%, 40%, 50%, 60%, 80% and 100% buffer B are used to elute peptides from the column. The resulting peptide fractions are then diluted 5× with 5% acetic acid. If necessary the sample volumes are reduced by placing them in a vacuum.

The mass spectrometric system used in this example is a hybrid quadrupole/time-of-flight mass spectrometer (Q-TOF-2, Waters/Micromass, Manchester UK). Samples eluted from the separatory column are introduced into the mass spectrometer through a nebulization-assisted electrospray interface (nano LC option for the Q-TOF-2). The data-dependent acquisition software of the mass spectrometer can be programmed to monitor masses provided in a list based on the presumed charge states and masses of the peptides included in the chimeric polypeptide, and presumably in the digested sample. When a candidate peptide is detected, the mass spectrometer can stop collecting MS data, and collect MS/MS data by passing only a narrow range of the available mass spectrum into a collision cell for fragmentation. Each peptide fraction that elutes from the separatory column can be similarly analyzed.

Presence of the enzymes of the purine nucleotide cycle in the protein samples will be determined by the presence of mass signals corresponding to the masses of one or more of the peptides for each enzyme that are shown in Table 4. Mass-shifted peaks for the labeled peptides from the chimeric polypeptide are then located. Relevant MS/MS data can also be assessed. A very reliable identification of the peptide of interest will be available when MS/MS data are available for both the sample peptide and the corresponding peptide standard. When the sample peptide is not observed, but the control peptide is observed, it is likely that the amount of the sample peptide is not detectable by the method.

The ratio of the mass signal intensities for the unlabeled and labeled versions of at least one tryptic cleavage peptide for each enzyme is determined and used to calculate the absolute amounts of the enzymes in the normal and cancerous samples. In general, the amount of a peptide generated by sequence-specific cleavage of a sample protein is equal to the amount of the sample protein prior to digestion. In other words, by determining the amount of a tryptic peptide derived from a protein of interest, the amount of the protein of interest is determined. In this example, the absolute amounts of the purine nucleotide enzymes in the samples are determined by comparing the mass signal intensities for the tryptic peptides generated from a given enzyme to the mass signal intensities of corresponding (identical sequence) isotopically-labeled peptides that are added to the sample in the form of the chimeric polypeptide and generated by tryptic digestion of the chimeric polypeptide. It is the ratio of signal intensities for the unlabeled (sample-derived) and labeled (chimeric polypeptide-derived) versions of the peptides that reflects the relative amounts of each in the sample. Of course, if the absolute amount of one of the peptides is known, the amount of the other can be determined using the ratio of mass spectral signals. Since the amount of the sample proteins is equal to the amount of the peptides derived from them, determination of the amounts of the sample-derived peptides is a determination of the amounts of the proteins in the sample.

In some embodiments, the ratio of mass spectral signals for the unlabeled to labeled versions of a peptide is multiplied by the known concentration of the chimeric polypeptide to provide a concentration for the protein of interest. Alternatively, the ratio of mass spectral signals for labeled to unlabeled peptides may be divided into the known concentration of the added chimeric polypeptide to provide a concentration of the protein of interest. For example, if the ratio of an unlabeled peptide signal for AMP deaminase to an labeled peptide signal for AMP deaminase in a given sample is 1.2, the absolute amount of AMP-deaminase in the sample is 100 pmol×1.2, or 120 pmol. If all six of the AMP-deaminase labeled/unlabeled mass spectral pairs are located (corresponding to the 6 tryptic peptides used in the chimeric polypeptide), an average ratio of the unlabeled and labeled mass signals for the peptides from a single protein of interest may be calculated and used to calculate the concentration of the protein of interest as above. Alternatively, the ratio of signals for each unlabeled/labeled pair is used to calculate a concentration of the protein of interest, and these are then averaged.

### EXAMPLE 3

### Selection of Protein Sets for a Single Chimeric Polypeptide

Mass tags for any number of particular different proteins may be combined to form a chimeric polypeptide (or a set of chimeric polypeptides). In some examples, the mass tags combined to form the chimeric polypeptide are selected based on a common property that they share, such as a grouping of target proteins that are to be spectroscopically analyzed. The mass tags, which may be one or more peptides that can be used to identify the different proteins of interest, are combined in a single chimeric polypeptide. The mass tags are generated by treatment of a protein of interest with a particular protein cleavage agent, and although it is possible to include multiple mass tags for a protein (such as generated by different protein cleavage agents) in a single chimeric polypeptide, each protein of interest will typically be represented by a single mass tag (which may be multiple peptides) in a single chimeric polypeptide. Such chimeric polypeptides can be expressed in a host cell grown on an isotopically-altered medium to provide standards for absolute quantitation of the proteins. The proteins for which mass tags are included in one or more chimeric polypeptides may be selected upon any number of criteria.

As mentioned before, one criterion, common property or shared characteristic upon which proteins may be grouped together for inclusion of their mass tags in a single chimeric polypeptide is according to the expected concentrations (amounts) of the proteins of interest in samples. Therefore, in one embodiment, mass tags for proteins that are expected to be present in a range of substantially similar concentrations (amounts) are combined. For example, mass tags for proteins that are expected to be present in concentrations (amounts) that are within 2 orders of magnitude of each other, for example, within 1 order of magnitude of each other, may be combined in a single chimeric polypeptide. Particular examples of concentration ranges of proteins for which mass tags may be combined in chimeric polypeptides are 1-10 pg/mL, 1-100 pg/mL, 10-100 pg/mL, 10-1000 pg/mL, 100-1000 pg/mL, 0.1-1 ng/mL, 0.1-10 ng/mL, 1-10 ng/mL, 1-100 ng/mL, 10-100 ng/mL, 10-1000 ng/mL, 0.1-1 μg/mL, 0.1-10 μg/mL, 1-10 μg/mL, 1-100 μg/mL, 10-100 μg/mL, 10-1000 μg/mL, 0.1-1 mg/mL, 0.1-10 mg/mL, 1-10 mg/mL, 1-100 mg/mL, 10-100 mg/mL, 10-1000 mg/mL, and 0.1-1 g/mL. Other, similar 1-2 orders of magnitude concentration ranges that overlap with these are contemplated, as are concentration ranges expressed in other units of amount or concentration such as moles, molarity, molality, and normality. In general, however, it is desirable to have all peptides present at roughly equal concentrations that are appropriate for MS/MS analysis. Data analysis of the ratio of intensities of selected isotopomers of unlabeled and labeled peptides can also be used when high resolution data is available. For example, if a sample peptide is present in unexpected abundance where the main peak of an isotopic cluster is beyond the linear response of the instrument, smaller, less abundant peaks due to individual isotopomers may be used for the quantitation.

Another consideration for quantitation purposes is the availability of the sample. If the sample is available in sufficient quantity, peptide controls can be added at a range of concentrations to a series of aliquots of the sample to determine optimal quantitation conditions for a wide range of sample peptide concentrations. Analysis of a single ‘basis’ sample, which is available in excess, leads naturally to the creation of a useful quantity standard in which the concentration of selected peptides present in the sample is known through calibration with known standards. Other sample manipulations address the control peptide level problem in other ways. For instance chimeric polypeptides can be expressed with different designed mass offsets which are resolved and allow these same control peptides to be added to the experimental sample at different concentrations, which provide overlap coverage for the range of possible concentration observed for the sample peptides.

Another example of a shared characteristic used to select the mass tags in the chimeric polypeptide is a shared functional, structural, biochemical, or other biological property of the target proteins. For example, proteins for which mass tags are to be combined in a single chimeric polypeptide are selected to include a particular class of protein (for example, a collagen or fibrinogen). Alternatively the proteins of interest for which mass tags are combined in a single chimeric polypeptide may be grouped according to an enzyme class (such as oxidoreductases, transferases, hydrolases, lyases, isomerases and ligases) subclass (such as transferases that transfer sulfur-containing groups), or sub-subclass (such as sulfur-containing transferases that transfer co-enzyme A). For example, if the proteins of interest are all enzymes of a particular class (such as transferases or hydrolases) the mass tags may be expressed as a single chimeric polypeptide. If multiple classes of protein (such as multiple classes of enzymes) are to be analyzed, then separate mass tag chimera may be prepared for each class. The ENZYME Data Bank (ExPASY, Geneva, Switzerland) provides additional examples of enzyme subclasses and sub-subclasses that may be considered when grouping proteins.

Groupings according to protein or enzyme class (or some sub-class thereof) may be further sub-divided according to any other criterion used to group proteins of interest. For example, where proteins of multiple classes are to be analyzed, the proteins may be grouped such that proteins of each class that are expected to fall within a particular range of concentrations (such as within one or two orders of magnitude in concentration) are grouped for inclusion in a single chimeric polypeptide. Thus, multiple chimera, each comprising the mass tags for proteins of a particular class and concentration range, may be designed to cover all proteins of interest at their varying concentrations (amounts).

Still another basis on which proteins may be grouped is by the metabolic or signaling pathway(s) in which they participate. For example, enzymes involved in the regulation of carbohydrate metabolism may be selected, and mass tags for the enzymes combined in one or more chimera. Additional examples include proteins involved in photosynthesis, lipid metabolism, protein kinase cascades, apoptosis signaling pathways, mitogenic signaling pathways, or transcription of nucleic acids.

Yet another basis is the location of the proteins. For example, glycoproteins of a cell or nuclear membrane may be grouped together, or proteins found in a particular organelle may be grouped together. There also is some advantage in grouping neighboring peptides from a protein together in a chimeric polypeptide. In some experiments it can be expected that there may be uncertainty about the extent of cleavage available to a protein in a sample. By recreating the actual cleavage site which must be targeted in a protein by the cleavage agent in the chimeric polypeptide this issue can be addressed. In larger sets of chimeric polypeptides it is possible to group chimeric polypeptides together in sequence so that the cleavage sites recreate the immediate sequence features of some cleavage sites in target proteins. Reproductions of neighboring peptides of target proteins in chimerical polypeptides can also be used to address the poorly addressed problems related to non-specific digestion by “specific” cleavage agents and control for unusual sequence specificity that may occur due to uncharacterized specificity of known cleavage agents or trace contaminants of other cleavage agents in otherwise pure preparations of another cleavage agent.

In general any combination of properties of the proteins of interest may be used to decide which mass tags will be combined in a single chimeric polypeptide, and how many different chimeric polypeptides will be needed to span the proteins of interest in a sample. For example, an important property for combining mass tags in a single chimeric polypeptide is a similarity in the expected concentration of the proteins of interest from which the mass tags are derived. Conversely, a dissimilarity in the concentrations of the proteins of interest (such as a greater than 2, 3 or 4 orders of magnitude in their concentration in a sample) may be used to group mass tags for the proteins of interest into multiple separate chimera. Thus, it is possible to use several chimera, each chimera including the mass tags for proteins of interest that occur in an expected narrow concentration range (such as within 1 or 2 orders of magnitude in concentration), to provide isotopically-labeled peptide standards for proteins of interest that span the range of possible protein concentrations in the sample.

### EXAMPLE 4

### An Exemplary Chimeric Polypeptide

A protein sequence was designed that would provide a means of examining a variety of outcomes that can occur when using a robotic in-gel digester, followed by LC/MS/MS to do protein identification. The following properties/features were incorporated into a set of five peptides: (1) the state of methionine oxidation (e.g., peptide T5), (2) the extent of Asp-Pro bond cleavage (e.g. peptide T2), (3) asparagine deamidation in labile sequences, (4) the chemical state of cysteine residues (e.g., peptide T3), (5) a low avidity peptide that serves as a control for the actual acetonitrile concentration present in the loaded sample (e.g., peptide T4), (6) a highly hydrophobic sequence that is used to assess the effectiveness of extraction of peptides from polyacrylamide gel fragments (e.g. peptide T6), (7) a spread of expected m/z's (e.g., 370 to 1232) that can provide for internal recalibration by preprocessing (see Gentzel et al., Proteomics 3:1597-1610, 2003), and (8) a spread of predicted elution times to provide semi-continuous presence of a control peptide to provide periodic verification of the performance of the mass spectrometer.

During the design of the chimeric polypeptide, the individual peptides were compared to the BLAST database (National Institutes of Health, Bethesda, Md.) to insure that the each peptide sequence did not produce a significant hit. The five individual peptides are shown in Table 5. The peptides were combined into a polypeptide sequence and the sequence submitted to a downloaded version of the DNAWorks 1.1 program (Hoover and Lubkowski, Nucleic Acids Res. 30:e43, 2002). Due to partial sequence homology of some of the peptides, manual changes were made in the sequence of a few of the DNA primers after the initial primers did not create the correct PCR product (however, in version 2.1 of the DNAWorks program, the potential for such mispriming becomes part of the sequence optimization algorithm).

TABLE 5

| | | SEQ |
|---|---|---|
| | | ID |
| Peptide Sequence | Peptide # | NO: |
| ATDESDPINGFIYYTTYTYTK | T2 | 20 |
| ATDEWIGGNFCTSYIIK | T3 | 21 |
| ADEIYK | T4 | 22 |
| ADEMYTISTIK | T5 | 23 |
| ATDESWIIIYYPTDYTYTK | T6 | 24 |

The synthetic gene was designed with 5′ and 3′ flanking sites to support rapid and efficient cloning into a directional-TOPO plasmid product, which generates an N-terminal His-tag polypeptide (Invitrogen, San Diego, Calif.).

(SEQ ID NO: 25)

CACC CGT GCC ACG GAC GAA TCT GAC CCG ATC AAC GGT
TTC ATC TAC TAC ACC ACC TAC ACC TAC ACC AAA GCG
ACC GAC GAA TGG ATC GGT GGT AAC TTC TGC ACC TCT
TAC ATC ATC AAA GCG GAC GAA ATC TAC AAA GCG GAC
GAA ATG TAC ACC ATC TCT ACC ATC AAA GCG ACC GAT
GAG TCC TGG ATC ATT ATC TAT TAC CCG ACC GAC TAT
ACG TAC ACT AAG GCG CCG CAC TAA

The sequence of the expressed chimeric protein with the vector originating His-tag and linker sequence (italics), designed peptides (underlined), and terminal three amino acid sequence (placed so that the terminal peptide is also released from the full sequence upon trypsin digestion) is:

(SEQ ID NO: 26)

MHHHHHHGKPIPNPLLGLDSTENLYFQGIDPF T R ATDESDPINGFIY
YTTYTYTK ATDEWIGGNFCTSYIIK ADEIYK ADEMYTISTIK ATDE
SWI IIYYPTDYTYTK APH

The polypeptide was overexpressed in a bacterial strain based on the T7 system and formed inclusion bodies. The inclusion bodies were partially separated from other cellular debris after sonication and low speed centrifugation, and solubilized with GdnHCl. The polypeptide was purified to a relatively high level of homogeneity using denatured IMAC (Qiagen, Valencia, Calif.), run on an SDS/PAGE gel, and excised. Following trypsin processing, the peptides were subjected to mass spectral analysis to determine if the designed properties/features were achieved.

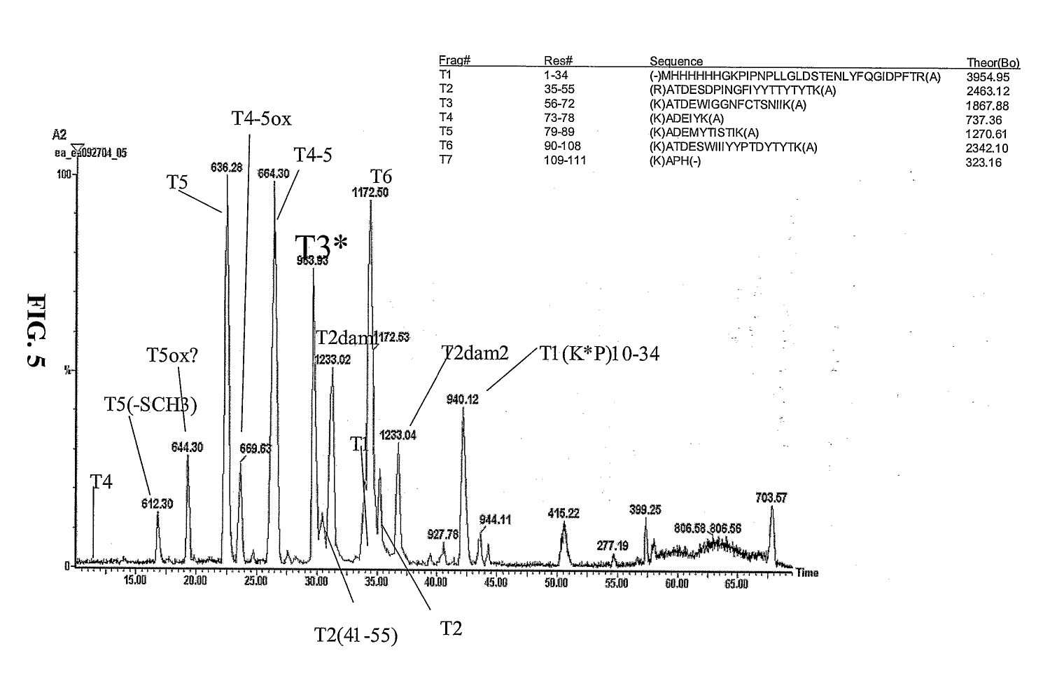

FIG. 5 is a base-peak intensity trace of an LC/MS experiment using the designed peptides. Using the data this trace is based on, as well as additional MS/MS data, the identifications of the peptides were made as shown in the figure. The T3 peptide (SEQ ID NO: 21) was found to have a Y N substitution at position 14, which was detectable in the MS/MS data. Subsequent electrospray MS of the whole protein (with no cysteine modification) matched the mass of the designed polypeptide (SEQ ID NO: 26) with the Y N residue change.

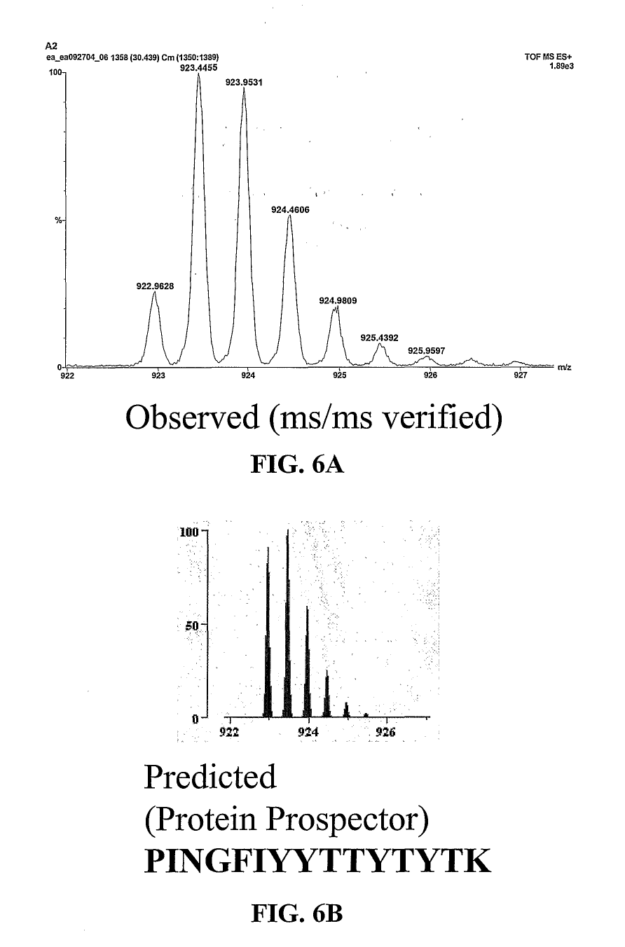

FIG. 6 illustrates the sequence verified position of a peptide that originates from the Asp-Pro bond cleavage (residues 6 and 7) in the T2 peptide (SEQ ID NO: 20). FIG. 6A is the product of spectral summation of an approximate 30 second interval of data obtained while the peptide was eluting into the mass spectrometer. The identity of the peptide was determined by MS/MS data to originate from Asp-Pro bond cleavage (residues 6 and 7) in the T2 peptide (SEQ ID NO: 20). FIG. 6B is a simulation of the expected abundance of different peaks expected in the mass spectrum. The PINGFIYYTTYTYTK peptide (residues 7-21 of SEQ ID NO: 20) is a result of Asp-Pro bond cleavage and the difference between the observed and predicted mass spectra is due to asparagines deamidation.

### EXAMPLE 5

### Mass Tags

In some embodiments, the mass tags that are included in the disclosed chimeric polypeptides are selected by mass spectrometric analysis of global protein digests of samples of interest (that contain any number of sets of proteins of interest) to identify one or more peptides that identify the proteins of interest in such digests. An exemplary two-stage technique for identification of mass tags for particular proteins is described by Smith et al. (see, Smith et al., Proteomics 2:513-23, 2002). Briefly, a plurality of peptides are generated by digestion (for example, using the protein cleavage agents discussed in Example 6 below) and screened by liquid chromatography and tandem mass spectrometry to identify potential mass tags (PMT's), that is, a set of peptides that are confirmed to be from a particular protein by comparison of their MS/MS spectra to MS/MS spectral patterns in the SEQUEST database (The Scripps Research Institute, La Jolla, Calif.). SEQUEST converts the character-based representation of amino acid sequences in a protein to fragmentation patterns which are compared against the MS/MS spectrum generated from the target peptide. An algorithm initially identifies amino acid sequences in the database that match the measured mass of the peptide, compares fragment ions against the MS/MS spectrum, and generates a preliminary score for each amino acid sequence. A cross correlation analysis is then performed on the top 500 preliminary scoring peptides by correlating theoretical, reconstructed spectra against the experimental spectrum, and output results are displayed accordingly. Optionally, the mass tags can be validated as accurate mass tags (AMTs, single peptides that identify a protein) using Fourier transform ion cyclotron resonance (FT-ICR) MS. In another example, a digested protein sample is directly analyzed by LC-MS/MS using FT-ICR. Due to the high mass accuracy of FT-ICR MS/MS measurements, identified peptides having distinctive masses can be immediately assigned as AMTs.

In addition to providing the identity of the mass tags to be used, such data also can be used to suggest approximate concentrations to be used for introduction and combination of peptides in individual chimeric polypeptides. Coarse concentration estimates can be obtained from relative intensity measurements between peptides of similar but not identical sequence.

### EXAMPLE 6

### Protein Cleavage Agents

As part of the disclosed methods, sample proteins of interest are contacted with one or more protein cleavage agents that cleave the proteins at defined cleavage sites and generate smaller peptides. Subsets (including single peptides) of these smaller peptides are mass tags for the proteins of interest. The identity of a peptide(s) that is (are) a mass tag for a protein of interest depends upon the protein cleavage agent (or agents) used, since protein cleavage agents differ in their sequence specificities. The mass signals for the smaller peptides generated with a particular protein cleavage agent are then compared to the mass signals for the isotopically-labeled standards that are released from the chimeric polypeptides of the disclosure. Thus, the chimeric polypeptides of the disclosure are typically designed to release isotopically-labeled mass tags for the proteins of interest upon treatment with the same protein cleavage agent (or agents) used to generate the mass tags for the proteins of interest in a sample.

Typically, it is desirable that the proteins of interest are consistently cleaved at particular bonds to provide reproducible sets of mass tags for the proteins. Thus, the protein cleavage agent (or agents) that is used to generate mass tags from the proteins of interest and the chimeric polypeptides may be chosen to have a high fidelity in recognizing and cleaving particular amino acid sequences. For example, trypsin is an endoprotease with a high fidelity for cleaving amino acid sequences at the C-terminus of the positively charged amino acids arginine (R) and lysine (K).

Proteolytic cleavage of the sample proteins can be performed either prior to or after adding one or more chimeric polypeptides containing isotopically-labeled mass tags for the proteins of interest to the sample. In one embodiment, the sample and the chimeric polypeptide are combined and treated with one or more protein cleavage agents. Alternatively, the sample and the chimeric polypeptide are treated separately with the same protein cleavage agent(s) and then combined. Optionally, the sample proteins (with or without added mass tags) can be fractionated after proteolytic cleavage of the sample and prior to analysis.

Protein cleavage agents include both proteolytic enzymes (proteases) as well as chemical protein cleavage agents. In one embodiment, the protein cleavage agent is an endoprotease such as trypsin, chymotrypsin, endoprotease ArgC, endoprotease aspN, endoprotease gluC, endoprotease lysC or a combination thereof. Further examples of proteases are found in Table 6, below. The proteases can be used alone or in combination to generate proteolytic fragments of the sample proteins and to release the isotopically-labeled mass tags from the chimeric polypeptides. As stated above, trypsin generally cleaves a peptide sequence after (on the carboxy side) of positively charged arginine or lysine residues. Chymotrypsin typically cleaves amino acid sequences on the carboxy side of bulky hydrophobic residues such as phenylalanine (F), tyrosine (Y), and tryptophan (W). Endoprotease ArgC generally cleaves a peptide sequence on the carboxy side of arginine (R) residues. Endoprotease aspN generally cleaves a peptide sequence on the carboxy side of asparagine (N) residues. Endoprotease lysC generally cleaves on the carboxy side of lysine (K) residues. The general sequence specificities of endoproteases are well known.

Alternatively (or in combination with proteolytic enzymes), the protein cleavage agent can include a chemical protein cleavage agent, such as cyanogen bromide, formic acid, or thiotrifluoroacetic acid. Cyanogen bromide, for example, generally cleaves a peptide sequence on the carboxy side of methionine residues.

Optionally, the sample can also be treated to remove post-translational modifications such as phosphate groups or ubiquitin groups prior to subjecting the proteolytic peptides to MS.

TABLE 6
Endoproteases*

| | |
|---|---|
| Acylamino Acid-Releasing Enzyme | Endoproteinase Asp-N |
| Aminopeptidase M | Endoproteinase Glu-C |
| Atrolysin C | Endoproteinase Lys-C |
| Bromelain | Enterokinase |
| Calpain I | N-Glycosidase F |
| Calpain II | Isopeptidase T |
| Carboxypeptidase A | Kallikrein |
| Carboxypeptidase B | MMP-1 |
| Carboxypeptidase W | MMP-2 |
| Carboxypeptidase Y | MMP-2/TIMP-2 Complex |
| Cathepsin B | MMP-3, Catalytic Domain |
| Cathepsin D | MMP-7 |
| Cathepsin G | MMP-8 |
| Cathepsin H | MMP-9 |
| Cathepsin L | MMP-9-Lipocalin Complex |
| Cathepsin S | MMP-9-Lipocalin-TIMP-1 |
| | Complex |
| Chymase | MMP-13 |
| α-Chymotrypsin | Papain |
| Chymotrypsin | Pepsin |
| Coagulation Factor Xa | Plasmin |
| Coagulation Factor α-XIIa | Proteinase K |
| Collagenase | Pyroglutamyl Aminopeptidase |
| Collagenase, Type I | Renin |
| Collagenase, Type III | Subtilisin A |
| Dansyl-Pepstatin | Thermolysin |
| Dipeptidylpeptidase IV | Thrombin |
| Dispase | Tissue Plasminogen Activator |
| Elastase | Trypsin |
| Endopeptidase, Neutral | Tryptase |
| Endoproteinase Arg-C | Urokinase |

*These proteases are available, for example, from Calbiochem, San Diego, CA.

### EXAMPLE 7

### Exemplary Mass Spectrometric Methods

Mass spectrometry, also called mass spectroscopy, is an instrumental approach that generates gas phase ions from a sample that are then separated and detected. The five basic parts of a typical mass spectrometer include: a vacuum system; a sample introduction device; an ionization source (which may be part of the sample introduction device); a mass analyzer; and an ion detector. A mass spectrometer determines the molecular weight of chemical compounds in the sample (and/or fragments thereof) by ionizing, separating, and measuring gas-phase ions according to their mass-to-charge ratio (m/z). Ions are generated in the ionization source by any number of processes including, for example, electron impact, protonation and deprotonation (such as in ESI), chemical ionization, fast-atom bombardment (FAB), surface enhanced laser desorption/ionization (SELDI) and matrix-assisted laser desorption/ionization (MALDI). Once ions are formed, they are directed into a mass analyzer and separated and detected according to their m/z. The separation of ions may be accomplished in any number of ways including, for example, passing the ions through a magnetic and/or electric field, capturing ions in an ion trap, or accelerating the ions in an electric field and separating them according to their time-of-flight as they pass through a field-free region. Examples of mass spectrometers that utilize one or more of these methods of ion separation methods include magnetic sector mass spectrometers (such as single, double and triple sector instruments), quadrupole mass spectrometers (Q), Fourier transform ion-cyclotron resonance mass spectrometers (FT-ICR), time-of-flight mass spectrometers (TOF), and combinations of these types of instruments (such as Q-TOF instruments). The ions detected following separation (and in some instances collisionally induced fragmentation, CID) provide information about the molecular weight and/or structure of the molecules in the introduced sample.

Although in some embodiments no physical simplification of a protein sample is necessary prior to collecting the mass spectral, in other embodiments, fractionation is desirable. Fractionation of a protein sample may be accomplished with any of a number of one-dimensional as well as multi-dimensional techniques known to one of skill in the art, including, for example, liquid chromatography (plate, column, capillary or high-pressure), reverse phase liquid chromatography (plate, column, capillary and/or high-pressure), size exclusion chromatography (plate, column, capillary and/or high-pressure), ion exchange chromatography (plate, column, capillary and/or high-pressure), affinity chromatography (plate, column, capillary and/or high-pressure), capillary electrophoresis, 1D or 2D gel electrophoresis, isoelectric focusing, free flow electrophoresis and selective adsorption (such as on a SELDI chip). Furthermore, combinations of these and other separation methodologies can be used to fractionate the sample into portions prior to analysis by MS. In one embodiment, capillary infusion is used to introduce a sample directly into a mass spectrometer following chromatographic or electrophoretic separation of a digested protein sample on a capillary column.

A particular method for that is suitable for introducing and ionizing protein samples (or fractions thereof) for mass spectral analysis is electrospray ionization (ESI). The electrospray ionization method is also particularly suited for direct coupling of chromatographic and/or electrophoretic separations with mass spectral analysis. In a typical ESI method, a liquid sample is introduced into the mass spectrometer through a metal capillary (or hollow needle) held at a high electrical potential of up to several kilovolts (for example, from about 500 V to about 4000 V). As sample passes through and out of the metal capillary, the molecules in the sample are de-solvated and ionized. Desolvation can be facilitated, for example, by interacting solvated ions with a countercurrent flow (for example, 6-9 L/min) of a heated gas before the ions enter into the vacuum of the mass analyzer. An ESI interface may also include one or more skimmers that reduce the amount of sample (and solvent) that actually enters the mass spectrometer.

Another particularly suitable method for protein and peptide sample ionization for mass spectral analysis is Matrix Assisted Laser Desorption/Ionization (MALDI). In MALDI, nonvolatile molecules (such as peptides and/or proteins) are embedded in a solid or crystalline “matrix” of laser light-absorbing molecules. Upon photonic excitation of the matrix with a laser of appropriate wavelength, the sample is desorbed from the solid phase directly into the gaseous phase and molecules in the sample are ionized. The ions are then accelerated and introduced into a mass spectrometer (typically, a TOF mass analyzer). The “matrix” is typically a small organic acid (such as cinnapinic acid) that is mixed in solution with the analyte in a 10,000:1 molar ratio and added to a sample stage onto which the laser light is directed. The matrix solution can be adjusted to neutral pH before mixing with the analyte. The MALDI ionization surface of the stage may be composed of an inert material or modified to actively capture an analyte. For example, an analyte binding partner may be bound to the surface to selectively absorb a target analyte or the surface may be coated with a thin nitrocellulose film for nonselective binding to the analyte. Alternatively, the surface may also be used as a reaction zone upon which the analyte is chemically modified (for example, cyanogen bromide degradation of protein; see, for example, Bai et al., Anal. Chem. 67:1705-10, 1995). Metals such as gold, copper and stainless steel are typically used as the substrate for the MALDI ionization stage. However, other commercially-available inert materials (for example, glass, silica, nylon and other synthetic polymers, or agarose or other carbohydrate polymers) can be used where it is desired to use the surface as a capture region or reaction zone. Additional information regarding the MALDI technique may be found, for example, in Lewis et al., “Matrix-assisted Desorption/Ionization Mass Spectrometry in Peptide and Protein Analysis,” Encyclopedia of Analytical Chemistry, Meyers (ed.), pp. 5880-5894, John Wiley and Sons Ltd, 2000).

Yet another particular method for sample ionization is Surface Enhanced Laser Desorption/Ionization (SELDI). SELDI is most often used in conjunction with a time-of-flight (TOF) mass spectrometer. SELDI is similar to MALDI in that the sample is added to a stage onto which laser light is directed to initiate desorption and ionization of sample molecules. The SELDI stage may incorporate modified surface chemistries that selectively adsorb certain analyte molecules from a sample, or the surface may be derivatized with energy-absorbing molecules that are not desorbed with the sample. Suitable SELDI stages (or “chips”) for protein and peptide analysis are available from Ciphergen Biosystems, Inc. (Fremont, Calif.). Additional information regarding the SELDI method may be found, for example, in U.S. Pat. No. 5,719,060 and PCT publication WO 98/59361.

Tandem mass spectrometry may also be employed. Tandem mass spectrometry (or MS/MS) may be used for peptides that cannot be identified directly by their characteristic mass (for example, because the mass spectrometer's resolution is insufficient to unambiguously differentiate two or more peptides by mass). This method combines two consecutive stages of mass analysis (such as by quadrupole mass analysis followed by time-of-flight mass analysis) to detect secondary fragment ions that are formed from a particular precursor ion. The first stage serves to isolate a particular ion of a particular peptide of interest based on its m/z. The second stage is used to analyze the product ions formed by spontaneous or induced fragmentation of the selected ion precursor. Between the stages, peptide fragment ions are produced from the precursor ion. Fragmentation can be achieved by a process known as collision-induced dissociation (CID), which is also known as collision-activated dissociation (CAD). A collision gas (typically Argon, although other noble gases can also be used) is introduced into a collision cell located between the two mass analyzer, and selected ions collide with the argon atoms, resulting in fragmentation. The fragments can then be analyzed in the second stage of mass analysis to obtain a fragment ion spectrum. Fragmentation of peptides and its use to identify peptide sequences by mass spectrometry has been well described (see, for example, Falick et al., J. Am Soc. Mass Spec. 4:882-93, 1993).

Still another method is the Fourier-transform ion cyclotron resonance method (FT-ICR). Very high mass accuracies may be attained by this method, so peptides (and the proteins from which they are derived) may often be identified directly from the measured mass of a single peptide. An FT-ICR mass spectrometer is a high-frequency mass spectrometer in which the cyclotron motion of ions having different m/z ratios in a magnetic field is exploited. The ions are excited by a pulse of radio-frequency electric field applied perpendicularly to the magnetic field. The excited cyclotron motion of the ions is subsequently detected as a time-domain signal, which is then Fourier-transformed into a frequency domain signal. The inverse relationship between frequency and the m/z ratio is used to convert the frequency domain signal into a mass spectrum. Application of the FT-ICR method to proteomic analysis (and the other methods discussed above) has been reviewed by Aebersold and Goodlett (Aebersold and Goodlett, “Mass Spectrometry in Proteomics,” Chem. Rev., 101: 269-295, 2001).

### EXAMPLE 8

### Alternative Expression Systems

In addition to the E coli expression system outlined in example 1, chimeric polypeptides may be expressed in other host cells, including yeast, viruses and mammalian cell lines, by using alternative cloning systems. Additional examples of commercially available expression systems include the ViraPower™ Lentiviral Expression System (Invitrogen, San Diego, Calif.), the ESP® yeast protein expression system (Stratagene, La Jolla, Calif.), the CompleteControl® mammalian expression system (Stratagene, La Jolla, Calif.) and the BD BacPack™ baculovirus expression system for insect host cells (BD Biosciences, Palo Alto, Calif.).

### EXAMPLE 9

### Kits

The isotopically-labeled mass tags disclosed herein can be supplied in the form of kit for use in mass-spectrometry-based quantitative proteomics. The kits may include undigested mass tag-containing chimeric polypeptides, or one or more individual mass tags previously released from a chimeric polypeptide by treatment with a protein cleavage agent. Such chimeric polypeptides or individual mass tags can be labeled or unlabeled. In such a kit, one or more of the mass tags is provided in one or more containers. Peptide mass tags can be provided suspended in an aqueous solution containing urea, GdnHCl, or other protein denaturant, frozen in a solution, or as a lyophilized powder. The container(s) in which the peptide mass tag(s) are supplied can be any conventional container that is capable of holding the supplied form; for example, microfuge tubes, ampoules, or bottles. In some applications, peptide mass tags can be provided in pre-measured single use amounts in individual, typically disposable, tubes or equivalent containers.

In one embodiment, kits are supplied with instructions. In one specific, non-limiting example, the instructions are written instructions. In another such example, the instructions are stored on a videocassette or on a CD. The instructions may, for example, inform the user of the proteins that may be quantified using the kit, and may instruct the user how to use the mass tags to quantitatively measure proteins of interest in a complex protein mixture via MS.

It should be understood that the foregoing relates only to particular embodiments and that numerous modifications or alterations may be made without departing from the true scope and spirit of the invention as defined by the following claims.

1. A method for high-throughput quantitative mass spectrometric analysis of a protein sample, comprising:
providing a sample comprising a known amount of multiple different mass tag sequences cleaved from a chimeric polypeptide comprising different mass tag sequences for two or more different target proteins that are separable by a protein cleavage agent at one or more protein cleavage sites, and an unknown amount of corresponding target mass tag sequences cleaved from target proteins in the sample, wherein the mass tag sequences cleaved from the chimeric polypeptide and the mass tag sequences cleaved from the target proteins are cleaved with the same protein cleavage agent, wherein the cleaved mass tag sequences from the chimeric polypeptide have an amino acid sequence that is substantially identical to the corresponding target mass tag sequences cleaved from the target proteins, and wherein the mass tag sequences cleaved from the chimeric polypeptide and the mass tag sequences cleaved from the target proteins are isotopic analogs of each other that are distinguishable by mass spectrometry;
performing mass spectrometry on the sample that comprises the mass tag sequences from the chimeric polypeptide and the mass tag sequences from the target proteins to provide a mass spectrum; and
using mass spectral signals in the mass spectrum for the known amounts of the mass tag sequences cleaved from the chimeric polypeptide and mass spectral signals for unknown amounts of corresponding target mass tag sequences to determine concentrations or amounts of the target proteins in the sample.

2. The method of claim 1, further comprising identifying a target protein by comparing the mass of one or more target mass tag sequences cleaved from the target protein by the protein cleavage agent with a database comprising masses of peptides generated by digestion of known peptides or proteins using the protein cleavage agent.

3. The method of claim 1, further comprising determining amounts of target proteins in the sample using ratios of mass spectral signals in the mass spectrum for the known amounts of the mass tag sequences cleaved from the chimeric polypeptide and the unknown amounts of corresponding target mass tag sequences cleaved from the target proteins.

4. The method of claim 3, wherein cleavage of both the chimeric polypeptide and the target proteins with the same protein cleavage agent provides corresponding pairs of mass tag sequences from the chimeric polypeptide and from the target proteins that have identical amino acid sequences.

5. The method of claim 3, wherein either the mass tag sequences of the target proteins or the mass tag sequences of the chimeric polypeptide are isotopically-altered with an isotope to provide mass tag sequences of the target proteins that are detectable by mass spectrometry as distinct from the mass tag sequences of the chimeric polypeptide.

6. The method of claim 5, wherein the isotope comprises a heavy stable isotope, and wherein the heavy stable isotope is ¹⁸O, ¹⁵N, ¹³C or ²H.

7. The method of claim 3, wherein the mass tag sequences of the chimeric polypeptide have masses that differ by predictable mass differences from masses for corresponding target mass tag sequences of the target proteins.

8. The method of claim 7, wherein the predictable mass differences are determined by incorporation of stable heavy isotopes into predictable numbers of sites in either the chimeric polypeptide or the target proteins.

9. The method of claim 5, wherein the protein cleavage agent recognizes and cleaves identical protein cleavage sites in the chimeric polypeptide and in the target protein, and cleaves the isotopically-labeled mass tag sequences from the target proteins or the chimeric polypeptide to form separated isotopically-labeled mass tag sequences.

10. A method for high-throughput quantitative mass spectrometric analysis of a protein sample, comprising:
treating a known amount of an isotopically-labeled chimeric polypeptide with a protein cleavage agent, wherein the chimeric polypeptide comprises mass tag sequences for two or more different target proteins that are separated by one or more protein cleavage agent sites and treating with the protein cleavage agent releases isotopically-labeled mass tag sequences for the two or more different target proteins from the isotopically-labeled chimeric polypeptide;
treating the protein sample with the same protein cleavage agent used to treat the isotopically-labeled chimeric polypeptide to provide a digested protein sample comprising target mass tag sequences of the target proteins;
adding the known amount of the treated chimeric polypeptide comprising isotopically-labeled mass tag sequences for the two or more different target proteins to the digested protein sample to provide a combined sample; and
analyzing the combined sample by mass spectrometry to provide a mass spectrum.

11. A method for high-throughput quantitative mass spectrometric analysis of a protein sample, comprising:
adding a known amount of a chimeric polypeptide to the protein sample to provide a combined sample, the chimeric polypeptide comprising mass tag sequences for two or more different target proteins that are separated by one or more protein cleavage agent sites, where the chimeric polypeptide is isotopically-labeled;
treating the combined sample with a protein cleavage agent that cleaves the chimeric polypeptide at the one or more protein cleavage agent sites and cleaves target proteins in the protein sample at one or more intrinsic protein cleavage agent sites recognized by the protein cleavage agent; and
analyzing the combined sample by mass spectrometry to provide a mass spectrum.

12. A method of making standards for quantitative proteomics, comprising:
providing a host cell;
expressing in the host cell a chimeric polypeptide that comprises different mass tag sequences for two or more different target proteins that are separable by a protein cleavage agent at one or more protein cleavage sites;
isolating the chimeric polypeptide from the host cell; and
treating the isolated chimeric polypeptide with the protein cleavage agent, wherein the protein cleavage agent cleaves the chimeric polypeptide at the protein cleavage sites to provide separated isotopic analogs of the target mass tag sequences of the target protein that are produced by treating the target protein with the protein cleavage agent.

13. The method of claim 12, wherein the host cell is grown in a medium and the chimeric polypeptide is expressed with an isotope of the medium incorporated into the mass tag sequences to provide isotopic analogs of the target mass tag sequences of the target proteins, wherein the mass tag sequences with the incorporated isotope are detectable as distinct from the target mass tag sequences of the target proteins by mass spectrometry.

14. The method of claim 12, further comprising reacting the mass tag sequences of the chimeric polypeptide with a covalent modification reagent, the covalent modification reagent comprising an isotope such that the reacted mass tag sequences of the chimeric polypeptide are isotopic analogs of target mass tag sequences of the target proteins that have been reacted with a corresponding covalent modification reagent and are detectable as distinct from the reacted mass tag sequences of the target protein by mass spectrometry.

15. The method of claim 12, wherein the protein cleavage sites are cleaved by a protein cleavage agent that cleaves the chimeric polypeptide to provide mass tag sequences having identical amino acid sequences as corresponding target mass tags from the target proteins.

16. The method of claim 12, wherein the protein cleavage agent is an endoprotease.

17. The method of claim 16, wherein the endoprotease is trypsin, and the protein cleavage sites are trypsin cleavage sites.

18. The method of claim 13, wherein the medium is isotopically-altered, and wherein the isotope in the isotopically altered medium is a stable heavy isotope that is present in the medium in greater abundance relative to its natural isotopic abundance, such that the isotopic analogs of the mass tag sequences in the chimeric polypeptide are detectable as distinct from the target mass tag sequences of the target protein by mass spectrometry.

19. The method of claim 12, further comprising cleaving the chimeric polypeptide with a protein cleavage agent that separates the chimeric polypeptide into multiple mass tag sequences that substantially uniquely identify corresponding target proteins by mass spectrometry, but wherein each mass tag sequence of the chimeric polypeptide has a mass that differs from a mass of corresponding target mass tag sequences of the target protein by a predictable mass difference.

20. The method of claim 19, wherein the predictable mass differences is determined by selecting an isotope that is incorporated into a predictable number of sites in the chimeric polypeptide or the target protein.

21. The method of claim 12, wherein the protein cleavage sites are chemical protein cleavage agent sites.

22. The method of claim 21, wherein the chemical protein cleavage agent sites are cyanogen bromide cleavage sites.

23. The method of claim 12, wherein the mass tag sequences of the two or more target proteins are fragments of proteins present in a biological sample at substantially similar concentrations and the mass tag sequences are mass tag sequences that identify the fragments in the biological sample by having sequences that are uniquely associated with the target proteins.

24. The method of claim 13, wherein the isotope for isotopically labeling is ¹⁵N present in the medium.

25. The method of claim 13, wherein the isotope for isotopically labeling is present in an isotopically-altered amino acid in the medium.

26. The method of claim 12, further comprising determining a concentration of the chimeric polypeptide.

27. The method of claim 26, further comprising quantitating the chimeric peptide, so that the quantitated chimeric peptide can be added to a biological sample in a predetermined amount that permits quantitation of the two or more target proteins in the biological sample when the target proteins are treated with the protein cleavage agent and analyzed by mass spectrometry.

28. The method of claim 12, wherein the target proteins share a common property.

29. The method of claim 28, wherein the common property comprises a common structure or functional characteristic.

30. The method of claim 29, wherein the common functional characteristic is activity in a common pre-selected biochemical pathway.

31. An isolated chimeric polypeptide prepared by the method of claim 12.

32. A set of spectrometric mass tag sequences comprising chimeric polypeptide cleavage products that are isotopic analogs of a corresponding set of target mass tag sequences from pre-selected target proteins.

33. The spectrometric mass tag sequences of claim 32, wherein the chimeric polypeptide is designed to produce cleavage products that differ form the corresponding set of target mass tag sequences from pre-selected target proteins by a predictable mass difference.

34. The spectrometric mass tag sequences of claim 32, wherein the target proteins share a common property.

35. A kit for performing high-throughput quantitative mass spectrometric analysis of a protein sample, comprising:
a chimeric polypeptide that comprises different mass tag sequences for two or more different target proteins where the target proteins correspond to and are identified by the different mass tag sequences, wherein each mass tag sequence of the chimeric polypeptide comprises an isotopic analog of a corresponding mass tag sequence of its target protein that is detectable by mass spectrometry as distinct from the mass tag sequence of the target protein, wherein the mass tag sequences of the chimeric polypeptide are separable from each other by a protein cleavage agent at one or more protein cleavage sites in the chimeric polypeptide; and
instructions for using the chimeric polypeptide to predict presence and/or quantities of target proteins present in the sample.

36. The kit of claim 35, wherein the chimeric polypeptide is provided in a known concentration.

37. The kit of claim 35, wherein the chimeric polypeptide is isotopically-labeled.


SEQUENCE LISTING (26 sequences)

SEQ ID NO: 1 - protein, 457 aa, Homo sapiens
MSGTRASNDRPPGAGGVKRGRLQQEAAATGSRVTVVLGAQWGDEGKGKVVDLLATDADIISRCQGGNNAGHTVVVDGKEYDFHLLPSGIINTKAVSFIGNGVVIHLPGLFEEAEKNEKKGLKDWEKRLIISDRAHLVFDFHQAVDGLQEVQRQAQEGKNIGTTKKGIGPTYSSKAARTGLRICDLLSDFDEFSSRFKNLAHQHQSMFPTLEIDIEGQLKRLKGFAERIRPMVRDGVYFMYEALHGPPKKILVEGANAALLDIDFGTYPFVTSSNCTVGGVCTGLGIPPQNIGDVYGVVKAYTTRVGIGAFPTEQINEIGGLLQTRGHEWGVTTGRKRRCGWLDLMILRYAHMVNGFTALALTKLDILDVLGEVKVGVSYKLNGKRIPYFPANQEMLQKVEVEYETLPGWKADTTGARRWEDLPPQAQNYIRFVENHVGVAVKWVGVGKSRESMIQLF

SEQ ID NO: 2 - protein, 480 aa, Homo sapiens
MPLFKLPAEEKQIDDAMRNFAEKVFASEVKDEGGRQEISPFDVDEICPISHHEMQAHIFHLETLSTSTEARRKKRFQGRKTVNLSIPLSETSSTKLSHIDEYISSSPTYQTVPDFQRVQITGDYASGVTVEDFEIVCKGLYRALCIREKYMQKSFQRFPKTPSKYLRNIDGEAWVANESFYPVFTPPVKKGEDPFRTDNLPENLGYHLKMKDGVVYVYPNEAAVSKDEPKPLPYPNLDTFLDDMNFLLALIAQGPVKTYTHRRLKFLSSKFQVHQMLNEMDELKELKNNPHRDFYNCRKVDTHIHAAACMNQKHLLRFIKKSYQIDADRVVYSTKEKNLTLKELFAKLKMHPYDLTVDSLDVHAGRQTFQRFDKFNDKYNPVGASELRDLYLKTDNYINGEYFATIIKEVGADLVEAKYQHAEPRLSIYGRSPDEWSKLSSWFVCNRIHCPNMTWMIQVPRIYDVFRSKNFLPHFGKMLE

SEQ ID NO: 3 - protein, 484 aa, Homo sapiens
MAAGGDHGSPDSYRSPLASRYASPEMCFVFSDRYKFRTWRQLWLWLAEAEQTLGLPITDEQIQEMKSNLENIDFKMAAEEEKRLRHDVMAHVHTFGHCCPKAAGIIHLGATSCYVGDNTDLIILRNALDLLLPKLARVISRLADFAKERASLPTLGFTHFQPAQLTTVGKRCCLWIQDLCMDLQNLKRVRDDLRFRGVKGTTGTQASFLQLFEGDDHKVEQLDKMVTEKAGFKRAFIITGQTYTRKVDIEVLSVLASLGASVHKICTDIRLLANLKEMEEPFEKQQIGSSAMPYKRNPMRSERCCSLARHLMTLVMDPLQTASVQWFERTLDDSANRRICLAEAFLTADTILNTLQNISEGLVVYPKVIERRIRQELPFMATENIIMAMVKAGGSRQDCHEKIRVLSQQAASVVKQEGGDNDLIERIQVDAYFSPIHSQLDHLLDPSSFTGRASQQVQRFLEEEVYPLLKPYESVMKVKAELCL

SEQ ID NO: 4 - protein, 6 aa, Artificial Sequence
  Exemplary peptide mass tag.
LIISDR

SEQ ID NO: 5 - protein, 11 aa, Artificial Sequence
  Exemplary peptide mass tag.
LDILDVLGEVK

SEQ ID NO: 6 - protein, 15 aa, Artificial Sequence
  Exemplary peptide mass tag.
EYDFHLLPSGIINTK

SEQ ID NO: 7 - protein, 5 aa, Artificial Sequence
  Exemplary peptide mass tag.
FLSSK

SEQ ID NO: 8 - protein, 5 aa, Artificial Sequence
  Exemplary peptide mass tag.
DLYLK

SEQ ID NO: 9 - protein, 6 aa, Artificial Sequence
  Exemplary peptide mass tag.
LSIYGR

SEQ ID NO: 10 - protein, 6 aa, Artificial Sequence
  Exemplary peptide mass tag.
IYDVFR

SEQ ID NO: 11 - protein, 9 aa, Artificial Sequence
  Exemplary peptide mass tag.
NPFLDFLQK

SEQ ID NO: 12 - protein, 16 aa, Artificial Sequence
  Exemplary peptide mass tag.
YETWCYELNLIAEGLK

SEQ ID NO: 13 - protein, 9 aa, Artificial Sequence
  Exemplary peptide mass tag.
NALDLLLPK

SEQ ID NO: 14 - protein, 6 aa, Artificial Sequence
  Exemplary peptide mass tag.
LLANLK

SEQ ID NO: 15 - protein, 5 aa, Artificial Sequence
  Exemplary peptide mass tag.
AELCL

SEQ ID NO: 16 - protein, 99 aa, Artificial Sequence
  Chimeric polypeptide.
LIISDRLDILDVLGEVKEYDFHLLPSGIINTKFLSSKDLYLKLSIYGRIYDVFRNPFLDFLQKYETWCYELNLIAEGLKLLANLKNALDLLLPKAELCL

SEQ ID NO: 17 - DNA, 297 bp, Artificial Sequence
  Nucleic acid encoding a chimeric polypeptide.
ttgatcatttctgatagactcgatatactagacgttttaggtgaagtaaaagagtacgactttcatttactaccaagtggaattatcaatactaaatttctgagctcaaaagacctctacctgaagttgtcgatatatggcaggatttacgacgtgtttcgaaacccgttcctagatttcttacaaaaatatgagacatggtgctatgaattgaatcttatagcggaggggctcaagcttctcgccaacttaaagaatgcactagatcttttgcttcctaaggctgaactgtgtctg

SEQ ID NO: 18 - DNA, 303 bp, Artificial Sequence
  E. coli optimized nucleic acid encoding a chimeric polypeptide.
atgaagttgatcatatcagataggctagacattctagatgtactaggtgaggtgaaggagtatgactttcatttacttccgagtgggattatcaatactaaattcctgtcgtctaaagacctctatttgaaactgagcatttacggaagaatatacgatgtttttcgaaaccctttccttgattttctccaaaagtacgaaacatggtgctatgaattgaatttaatagctgagggcctcaagctccttgcaaacctaaaaaatgcgttagacctgcttctgccaaaagccgaattatgtttg

SEQ ID NO: 19 - DNA, 307 bp, Artificial Sequence
  E. coli optimized nucleic acid encoding a chimeric polypeptide,with additional cloning sequences.
caccatgaagttgatcatatcagataggctagacattctagatgtactaggtgaggtgaaggagtatgactttcatttacttccgagtgggattatcaatactaaattcctgtcgtctaaagacctctatttgaaactgagcatttacggaagaatatacgatgtttttcgaaaccctttccttgattttctccaaaagtacgaaacatggtgctatgaattgaatttaatagctgagggcctcaagctccttgcaaacctaaaaaatgcgttagacctgcttctgccaaaagccgaattatgtttg

SEQ ID NO: 20 - protein, 21 aa, Artificial Sequence
  Designed peptide for protein identification.
ATDESDPINGFIYYTTYTYTK

SEQ ID NO: 21 - protein, 17 aa, Artificial Sequence
  Designed peptide for protein identification.
ATDEWIGGNFCTSYIIK

SEQ ID NO: 22 - protein, 6 aa, Artificial Sequence
  Designed peptide for protein identification.
ADEIYK

SEQ ID NO: 23 - protein, 11 aa, Artificial Sequence
  Designed peptide for protein identification.
ADEMYTISTIK

SEQ ID NO: 24 - protein, 19 aa, Artificial Sequence
  Designed peptide for protein identification.
ATDESWIIIYYPTDYTYTK

SEQ ID NO: 25 - DNA, 241 bp, Artificial Sequence
  Nucleic acid encoding a chimeric polypeptide.
cacccgtgccacggacgaatctgacccgatcaacggtttcatctactacaccacctacacctacaccaaagcgaccgacgaatggatcggtggtaacttctgcacctcttacatcatcaaagcggacgaaatctacaaagcggacgaaatgtacaccatctctaccatcaaagcgaccgatgagtcctggatcattatctattacccgaccgactatacgtacactaaggcgccgcactaa

SEQ ID NO: 26 - protein, 111 aa, Artificial Sequence
  Chimeric polypeptide.
MHHHHHHGKPIPNPLLGLDSTENLYFQGIDPFTRATDESDPINGFIYYTTYTYTKATDEWIGGNFCTSYIIKADEIYKADEMYTISTIKATDESWIIIYYPTDYTYTKAPH